(19) United States
(12) Patent Application Publication
(10) Pub. No.: US 2022/0339099 A1
(43) Pub. Date: Oct. 27, 2022

(54) COMPOSITIONS OF INTERLEUKIN-1 RECEPTOR ANTAGONIST

(71) Applicant: Altavant Sciences GmbH, Basel (CH)

(72) Inventors: Stephen A. WRING, South Boston, VA (US); Jayne E. HASTEDT, San Carlos, CA (US); Kenneth Baker LEWIS, JR., Seattle, WA (US); Michelle PALACIOS, Garner, NC (US); Nani Putri KADRICHU, San Carlos, CA (US); Elvin Sanghwan LEE, Teaneck, NJ (US)

(21) Appl. No.: 17/726,164

(22) Filed: Apr. 21, 2022

Related U.S. Application Data
(60) Provisional application No. 63/244,807, filed on Sep. 16, 2021.
(60) Provisional application No. 63/178,062, filed on Apr. 22, 2021.

(51) Int. Cl. A61K 9/00; A61K 47/18; A61K 47/02; A61K 47/26; A61K 38/20

(52) U.S. Cl. CPC A61K 9/0078; A61K 47/183; A61K 47/02; A61K 47/26; A61K 38/2006

(57) ABSTRACT

A pharmaceutical composition is disclosed, including: an interleukin-1 receptor antagonist, wherein the interleukin-1 receptor antagonist is a protein or a peptide; a buffer; and optionally one or more additional components each selected from the group consisting of a stabilizer and a tonicity modifier, wherein the pharmaceutical composition is adapted for administration via inhalation. Kits including the pharmaceutical composition of same and method of using same for treating an inflammatory disorder of lower airways in a human subject are also disclosed.

### CROSS-REFERENCE TO RELATED APPLICATIONS

This application claims the benefit of and priority under 35 U.S.C. § 119(e) to U.S. Ser. No. 63/244,807 filed Sep. 16, 2021; and U.S. Ser. No. 63/178,062 filed Apr. 22, 2021; the contents of each of which are hereby incorporated by reference in their entireties.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction of the patent document or patent disclosure as it appears in the U.S. Patent and Trademark Office patent file or records, but otherwise reserves any and all copyright rights.

### INCORPORATION BY REFERENCE

All documents cited herein are incorporated herein by reference in their entirety.

### FIELD OF THE INVENTION

The invention relates generally to the field of pharmaceutical science. More particularly, the invention relates to compounds and compositions useful as pharmaceuticals for treating various lower airways disorders.

### BACKGROUND

### Bronchiolitis Obliterans Syndrome

Bronchiolitis Obliterans Syndrome (BOS) is a non-reversible lung disease characterized by obstruction of the small airways impeding airflow. BOS is one of the most common complications after lung or hematopoietic stem cell transplant and can also occur after exposure to inhaled toxins and gases. BOS patients experience shortness of breath, decreased ability to participate in daily activities, fatigue, and cough. BOS is diagnosed as a decline in lung function tests (FEV1) and confirmed with imaging (e.g., CT, X-ray). BOS disease progression involves activation of alloimmune responses, innate immune stimuli and infiltration of immune cells. Additionally, the lung repair pathway is aberrantly activated causing fibroblast proliferation, activation of smooth muscle surrounding the airways, and narrowing of the lumen within the terminal and distal bronchioles. There are no currently approved therapies by the U.S. Food and Drug Administration (FDA) for BOS. Other drugs that are in late-stage clinical development (e.g., inhaled cyclosporine) have a number of drawbacks, a history of failures and may not have the optimum mechanism (e.g., patients are typically already receiving a calcineurin inhibitor).

### Chronic Obstructive Pulmonary Disease

Chronic Obstructive Pulmonary Disease (COPD) is a major cause of morbidity and mortality worldwide. Overall health status and mortality are tightly associated with the severity of airflow obstruction. COPD is an inflammatory condition and neutrophil elastase has long been considered a significant mediator of the disease. Often, subjects are inadequately treated, resistant, or refractory to current therapies. COPD affects the peripheral airways and is associated with chronic irreversible obstruction of expiratory flow. This inflammatory disorder of the small airways includes chronic bronchitis (mucus hypersecretion with goblet cell and sub mucosal gland hyperplasia) and emphysema (destruction of airway parenchyma) associated with fibrosis and tissue damage.

### Toxic Inhalation Lung Injury

Toxic inhalation lung injury involves damage to the lung caused by toxic agents, e.g., chemicals and irritants, carried into the lower airways. Toxic inhalation lung injury can result in varying degrees of erythema, carbonaceous deposits, bronchorrhea, severe inflammation, copious carbonaceous deposits, and/or bronchial obstruction. In the most severe cases, there is evidence of mucosal sloughing, necrosis, and endoluminal obliteration. Toxic inhalation lung injury may be associated with a broad spectrum of respiratory disorders, depending on the inhaled toxic agent.

Toxic agents with different solubility and particle sizes have differential effects on different parts of the respiratory system. Inhaled toxins with high water solubility can localize in the upper airways, while those with low water solubility tend to localize more frequently in the lower airways. Toxins of larger particle size tend to localize in the upper airways, while those of smaller particle size penetrate the lower airways.

Current treatment strategies for toxic inhalation lung injury vary depending on the specific types of toxic agents involved, the duration of exposure, and the resulting extent of injury. Treatment options may include excision of burnt tissue along with skin graft replacement, administration of normobaric oxygen, mechanical ventilation, resuscitation, and/or administration of various pharmaceutical agents. See Dries et al., J. Trauma. Resuscitation. Emerg. Med., 2013, 21, 31-45.

Inhalation of diacetyl (Kreiss, K., et al., N. Engl. J. Med., 2002, 347, 330-338; Akpinar-Elci, M., et al., European Respiratory Journal, 2004, 24, 298-302), acetaldehyde and formaldehyde, as well as other volatile compounds, has been demonstrated as a cause of bronchiolitis obliterans (BO). Inhalation exposure to these compounds in the U.S. in more recent years has occurred at commercial-scale plants, such as coffee processing facilities and popcorn production plants, where processing workers can be at risk of exposure to airborne diacetyl (2,3-butanedione), 2,3-pentanedione, and/or 2,3-hexanedione, compounds which have been linked to BO.

### Vaping-Associated Lung Injury

Vaping-associated lung injury is a newly recognized specific group of syndromes under the general category of toxic-inhalation lung injury. The existing case studies demonstrate a heterogeneous collection of pneumonitis patterns that include acute eosinophilic pneumonia, organizing pneumonia, lipoid pneumonia, diffuse alveolar damage, diffuse alveolar hemorrhage, hypersensitivity pneumonitis, and/or giant-cell interstitial pneumonitis. Regardless of the specific pneumonitis pattern, pathophysiology of vaping-associated lung injury commonly includes inflammation, edema of airways, and acute lung damage. See Butt et al., N. Engl. J. Med., 2019, 318, 1780-1781.

Several toxic compounds have been identified in vaping products: flavorants (e.g., diacetyl), nicotine, carbonyls, volatile organic compounds (e.g., benzene and toluene), trace metal elements, α-Tocopheryl acetate, and bacterial endotoxins and fungal glucans. See Christiani, D., N. Engl. J. Med., Online Editorial, Sep. 6, 2019; available at https://www.nejm.org/doi/full/10.1056/NEJMe1912032#article_citing_articles. There are currently no standard guidelines for treatment of vaping-associated lung injury. Patients who are admitted to hospital are typically treated with antibiotics or steroids, but many continue to exhibit abnormalities at short-term follow-up periods. See Blagev et al., The Lancet, Nov. 8, 2019; available online at https://www.thelancet.com/journals/lancet/article/PHS0140-6736(19)32679-0/fulltext.

### Pulmonary Langerhans Cell Histiocytosis (PLCH)

Pulmonary langerhans cell histiocytosis (PLCH) is a rare disease affecting predominantly smokers. PLCH is a specific type of histiocytic syndrome characterized by accumulation of langerhans (antigen-presenting cells) and other inflammatory cells in small airways, resulting in the formation of nodular inflammatory lesions. More advanced stages are characterized by cystic lung destruction, cicatricial scarring of airways, and pulmonary vascular remodeling. PLCH often leads to death over a period of few years due to respiratory failure or malignancy. Current treatment includes corticosteroids, but it remains unclear whether this is an effective treatment option. See Murakami et al., Cell Communication and Signaling, 2015, 13, 1-15.

### Non-Cystic Fibrosis Bronchiectasis

Bronchiectasis is a heterogenous chronic lung disorder characterized by recurrent cough, sputum production, and recurrent respiratory infections. Over 95% of bronchiectasis is of the non-cystic fibrosis type. Mortality rate is significant, ranging from 10 to 16% over an approximate 4-year observation period. The pathology of the disease includes dilatation of the bronchi that lead to airway inflammation and chronic bacterial colonization. Treatment typically involves a multimodal approach that includes airway clearance, anti-inflammatory agents, and inhaled antibiotics. Some patients fail to adequately respond to any currently recognized therapeutic approach and may require lobectomy or segmentectomy. See Chalmers et al., Molecular Immunology, 2013, 55, 27-34.

### Diffuse Panbronchiolitis

Diffuse panbronchiolitis is characterized by chronic sinobronchial infection, peribronchial inflammation, and significant reduction in airflow. Symptoms include crackles, wheezes, productive cough, and chronic sinusitis. Diffuse panbronchiolitis is largely resistance to bronchodilators. The first-in-line treatment involves administration of macrolides which effectively inhibit bacterial growth and reduce inflammation but can lead to several adverse side effects. See Scambler et al., Immunology, 2018, 154, 563-573.

### Acute Respiratory Distress Syndrome (ARDS)

Acute Respiratory Distress Syndrome (ARDS) is a syndrome of acute respiratory failure with progressive arterial hypoxemia, dyspnea, and/or breathlessness. The pathogenesis of ARDS involves the accumulation of protein-rich and neutrophilic pulmonary edema in the lung coupled with significant inflammation. ARDS is life threatening and requires immediate endotracheal intubation and positive pressure ventilation to prevent lung failure. There is a potential role for pharmacological treatment coupled with ventilation such as the use of glucocorticoids, surfactants, inhaled nitric oxide, antioxidants, protease inhibitors, and a variety of other anti-inflammatory agents. However, the currently available treatment options for ARDS have not been shown to be sufficiently effective. See Park et al., Am. J. Respir. Crit. Care. Med., 2001, 164, 1896-1903.

### Reactive Airways Dysfunction Syndrome (RADS)

Reactive airways dysfunction syndrome (RADS) is a persistent asthma-like disorder with sudden onset following a single acute exposure to an inhaled irritant. Chemical irritants associated with RADS may include chlorine, toluene diisocyanate (TDI), nitrogen oxides, morpholine, sulfuric acid, ammonia, and phosgene. Conventional asthma treatments can be used in RADS, including corticosteroids and bronchodilators. However, currently there are no effective treatment options for patients with persistent RADS. See Shaken et al., Occupational Med., 2008, 58, 205-211.

### Bronchiolitis Obliterans Organizing Pneumonia (BOOP)

Bronchiolitis obliterans organizing pneumonia (BOOP) is a syndrome characterized symptomatically by subacute or chronic respiratory illness. Patients with BOOP may exhibit persistent nonproductive cough, effort dyspnea, low-grade pyrexia, and/or malaise. Pathologically, patients having BOOP may have granulation tissues in the bronchiolar lumen, alveolar ducts and alveoli, with variable degrees of inflammation. Current treatment options for BOOP are limited and typically involve oral corticosteroid therapy. A small number of patients who do not respond to standard treatment may require lung transplant. See Al-Ghanem et al., Ann. Thorac. Med., 2008, 3, 67-75.

### Pulmonary Arterial Hypertension (PAH)

Pulmonary arterial hypertension (PAH) is a chronic and progressive disease leading to right heart failure and ultimately death if untreated. Right heart failure can lead to fluid retention, hepatic congestion, ascites, and peripheral edema. Remodeling of small pulmonary arteries via the proliferation of smooth muscle and endothelial cells may play a major role in the pathogenesis of PAH. This abnormal proliferation includes hypertrophy of the media and intima, and the formation of tumor-like lesions from endothelial cells in regions of pulmonary artery bifurcation (plexiform lesions).

### Restrictive Allograft Syndrome (RAS)

Restrictive allograft syndrome (RAS) is a form of chronic lung allograft dysfunction (CLAD) after lung transplantation. The main characteristics of RAS include a persistent and unexplained decline in lung function and persistent parenchymal infiltrates. The median survival after diagnosis of RAS is 6 to 18 months, significantly shorter than other forms of CLAD. Treatment options are limited, as therapies used for BOS are typically ineffective at halting disease progression.

### Interstitial Lung Disease (ILD)

Interstitial lung disease (ILD) is an umbrella term used to refer to chronic lung disorders characterized by inflammation of the lung tissue progressively causing pulmonary scarring/fibrosis. Fibrosis may progressively cause lung stiffness, reducing the ability of the air sacs to capture and carry oxygen into the bloodstream and eventually leads to permanent loss of the ability to breathe.

### Idiopathic Pulmonary Fibrosis (IPF)

Idiopathic pulmonary fibrosis (IPF) is an age-related chronic and progressive lung disease of unknown cause that has few treatment options, and those only delay disease progression. IPF is characterized by radiographically evident interstitial infiltrates predominantly affecting the lung bases and by progressive dyspnea and worsening of pulmonary function.

### IL-1Ra

Anakinra is a recombinant human interleukin 1 receptor antagonist (rhIL-1Ra) which is a 17.2 kDa protein expressed in E. coli. It is sequence identical to human IL-1Ra protein with an additional methionine amino acid on the N-terminus. The protein belongs to the IL-1 fibroblast growth factor (FGF) family and is a naturally occurring IL-1 blocker that regulates inflammation. The protein structure is known as a β-trefoil which consists of 11 antiparallel β-strands, six of which are arranged in the form of a β-barrel end-closed by another six β-strands. See Murzin, A. G., Lesk, A. M., and Chothia, C. (1992) beta-Trefoil fold. Patterns of structure and sequence in the Kunitz inhibitors interleukins-1 beta and 1 alpha and fibroblast growth factors, J. Mol. Biol. 223, 531-543; Vigers, G. P. A., Caffes, P., Evans, R. J., Thompson, R. C., Eisenberg, S. P., and Brandhuber, B. J. (1994) X-ray structure of interleukin-1 receptor antagonist at 2.0-A resolution, J. Biol. Chem. 269, 12874-12879; Stockman, B. J., Scahill, T. A., Strakalaitis, N. A., Brunner, D. P., Yem, A. W., and Deibel, M. R., Jr. (1994) Solution structure of human interleukin-1 receptor antagonist protein, FEBS Lett. 349, 79-83; Schreuder, H. A., Rondeau, J. M., Tardif, C., Soffientini, A., Sarubbi, E., Akeson, A., Bowlin, T. L., Yanofsky, S., and Barrett, R. W. (1995). Refined crystal structure of the interleukin-1 receptor antagonist. Presence of a disulfide link and a cis-proline, Eur. J. Biochem. 227, 838-847. Proteins with predominant β-strand secondary structures are generally prone to aggregation. It has been demonstrated that interaction with the IL-1Ra positively charged site within the protein could control/suppress protein aggregation via interference with its self-association pathway. Researchers have shown that citrate has a lower aggregation rate than phosphate. See Raibekas, A. A.; Bures, E. J.; Siska, C. C.; Kohno, T.; Latypov, R. F.; Kerwin, B. A. Anion Binding and Controlled Aggregation of Human Interleukin-1 Receptor Antagonist. Biochemistry 2005, 44 (29), 9871-9879.

The generic name of the rhIL-1Ra is anakinra, and a subcutaneous (SC) formulation has been approved by FDA for indications including reducing signs and symptoms of moderately to severely active rheumatoid arthritis (Kineret™). There are no FDA-approved treatments for respiratory tract indications using rhIL-1Ra, and there are no FDA-approved formulations of rhIL-1Ra for inhaled delivery. Therefore, there remains a great need to develop safe and effective inhaled (e.g., nebulized, delivered via dry powder, etc.) formulations of rhIL-1Ra to treat a variety of lower airways disorders.

### SUMMARY OF THE INVENTION

In one aspect, described herein is a pharmaceutical composition comprising: an interleukin-1 receptor antagonist, wherein the interleukin-1 receptor antagonist is a protein or a peptide; a buffer; and optionally one or more additional components each selected from the group consisting of a stabilizer and a tonicity modifier, wherein the pharmaceutical composition is adapted for administration via inhalation.

In some embodiments, the buffer comprises an amino acid or phosphate. In some embodiments, the buffer comprises a positively charged amino acid. In some embodiments, the positively charged amino acid is selected from the group consisting of lysine, arginine, and histidine. In some embodiments, the positively charged amino acid is histidine. In some embodiments, the buffer is selected from the group consisting of citrate, phosphate, succinate, histidine, lysine, arginine, glutamate, pyrophosphate, 4-(2-hydroxyethyl)-1-piperazineethanesulfonic acid (HEPES), and a combination thereof. In some embodiments, the buffer comprises histidine or phosphate. In some embodiments, the pharmaceutical composition is a liquid composition comprising histidine in a concentration of between about 5 mM and 50 mM. In some embodiments, the concentration of histidine is about 5, 10, 15, 20, 25, 30, 35, 40 or 45 mM. In some embodiments, the concentration of histidine is about 10 mM. In some embodiments, the pharmaceutical composition is a liquid composition comprising phosphate in a concentration of between about 1 mM and 50 mM. In some embodiments, the concentration of phosphate is about 5, 10, 15, 20, 25, 30, 35, 40 or 45 mM. In some embodiments, the concentration of phosphate is about 10 mM.

In some embodiments, the pharmaceutical composition further comprises a stabilizer selected from the group consisting of a surfactant, a chelating agent, a sugar, and a combination thereof. In some embodiments, the stabilizer is a non-reducing sugar. In some embodiments, the non-reducing sugar is selected from the group consisting of trehalose, sucrose, glycerol, sorbitol, and a combination thereof. In some embodiments, the non-reducing sugar is trehalose. In some embodiments, the pharmaceutical composition is a liquid composition, and the concentration of the non-reducing sugar is greater than about 5% (w/v). In some embodiments, the pharmaceutical composition is a liquid composition comprising trehalose in a concentration of between about 100 mM and 350 mM. In some embodiments, the concentration of trehalose is about 115 mM. In some embodiments, the stabilizer is a chelating agent which is ethylenediaminetetraacetic acid (EDTA) disodium. In some embodiments, the pharmaceutical composition is a liquid composition comprising ethylenediaminetetraacetic acid (EDTA) disodium in a concentration of between about 0.05 mM and 1 mM. In some embodiments, the concentration of ethylenediaminetetraacetic acid (EDTA) is about 0.53 mM. In some embodiments, the stabilizer is a surfactant selected from the group consisting of polysorbate 80, polysorbate 20, polyoxyethylene(23) lauryl ether (Brij™ 35), sorbitan trioleate (Span™ 85), and a combination thereof. In some embodiments, the pharmaceutical composition is a liquid composition comprising polysorbate 80 in a concentration of between about 0.001% and 1% (w/v). In some embodiments, the concentration of polysorbate 80 is between about 0.05% and 0.035% (w/v).

In some embodiments, the pharmaceutical composition further comprises a tonicity modifier. In some embodiments, the tonicity modifier is selected from the group consisting of sodium chloride, mannitol, taurine, hydroxyproline, proline, and a combination thereof. In some embodiments, the pharmaceutical composition is a liquid composition comprising sodium chloride in a concentration of between about 10 mM and 50 mM. In some embodiments, the concentration of sodium chloride is about 20 mM. In some embodiments, the pharmaceutical composition comprises: a buffer comprising an amino acid or phosphate; and a stabilizer comprising a non-reducing sugar. In some embodiments, the composition comprises: a buffer comprising histidine or phosphate; and a stabilizer comprising trehalose. In some embodiments, the composition comprises: histidine; trehalose; sodium chloride; polysorbate 80; and ethylenediaminetetraacetic acid (EDTA) disodium. In some embodiments, the composition comprises: phosphate; trehalose; sodium chloride; polysorbate 80; and ethylenediaminetetraacetic acid (EDTA) disodium. In some embodiments, the pH of the liquid composition is between about 6 and 8. In some embodiments, the pH of the liquid composition is about 6.5. In some embodiments, the osmolality of the liquid composition is between about 200 mOsm/kg and 400 mOsm/kg. In some embodiments, the osmolality of the liquid composition is about 300 mOsm/kg. In some embodiments, the pharmaceutical composition is a liquid composition comprising the interleukin-1 receptor antagonist in a concentration of between about 1 mg/mL and 30 mg/mL. In some embodiments, the concentration of the interleukin-1 receptor antagonist is about 5 mg/mL. In some embodiments, the concentration of the interleukin-1 receptor antagonist is about 20 mg/mL. In some embodiments, the interleukin-1 receptor antagonist is anakinra.

In another aspect, described here is a kit comprising a pharmaceutical composition according to any one of the preceding claims and a delivery device suitable for direct administration of the pharmaceutical composition to the respiratory tract of a patient.

In some embodiments, the respiratory tract comprises the lower airways. In some embodiments, the delivery device is configured to deliver an effective amount of the pharmaceutical composition via inhalation. In some embodiments, the delivery device is selected from the group consisting of a nebulizer, an inhaler, and an aerolizer. In some embodiments, the delivery device is selected from the group consisting of a jet nebulizer, a mesh nebulizer, an ultrasonic nebulizer, a metered dose inhaler, and a dry powder inhaler. In some embodiments, the nebulizer is selected from the group consisting of the Aerogen Solo and the AeroEclipse II nebulizers. In some embodiments, the nebulizer is the Aerogen Solo. In some embodiments, the droplet size of the liquid composition produced by the delivery device is between about 0.5 μm and 10 μm in diameter. In some embodiments, the liquid composition produced by the delivery device is between about 2.5 μm and 4 μm in diameter. In some embodiments, the droplet size of the liquid composition produced by the delivery device is about 3.5 μm in diameter.

In another aspect, described here is a method of treating an inflammatory disorder of the respiratory tract comprising administering to a patient in need thereof the pharmaceutical composition according to any of the embodiments described herein.

In some embodiments, the inflammatory disorder of the respiratory tract is an inflammatory disorder of the lower airways. In some embodiments, the inflammatory disorder is selected from the group consisting of a toxic-inhalation lung injury, pulmonary langerhans cell histiocytosis, non-cystic fibrosis bronchiectasis, diffuse panbronchiolitis, acute respiratory distress syndrome (ARDS), reactive airways dysfunction syndrome (RADS), bronchiolitis obliterans organizing pneumonia (BOOP), bronchiolitis obliterans syndrome (BOS), interstitial lung disease (ILD), idiopathic pulmonary fibrosis (IPF), pneumonitis, primary graft dysfunction (PGD), restrictive allograft syndrome (RAS), pulmonary arterial hypertension (PAH), and reperfusion injury. In some embodiments, the toxic-inhalation lung injury is caused by inhalation of one or more chemical warfare agents. In some embodiments, the chemical warfare agent is selected from the group consisting of chlorine gas and sulfur mustard. In some embodiments, the toxic-inhalation lung injury is chlorine-induced bronchiolitis obliterans syndrome (BOS) and sulfur mustard-induced bronchiolitis obliterans syndrome (BOS). In some embodiments, the toxic-inhalation lung injury is caused by inhalation of one or more environmental and/or industrial toxic agents. In some embodiments, the environmental and industrial toxic agents are selected from the group consisting of isocyanate, nitrogen oxide, morpholine, sulfuric acid, ammonia, phosgene, diacetyl, 2,3-pentanedione, 2,3-hexanedione, fly ash, fiberglass, silica, coal dust, asbestos, hydrogen cyanide, cadmium, acrolein, acetaldehyde, formaldehyde, aluminum, beryllium, iron, cotton, tin oxide, bauxite, mercury, sulfur dioxide, zinc chloride, polymer fumes, and metal fumes. In some embodiments, the toxic-inhalation lung injury is pneumoconiosis or bronchiolitis obliterans. In some embodiments, the toxic-inhalation lung injury is a vaping-associated lung injury. In some embodiments, the vaping-associated lung injury is caused by inhalation of one or more agents selected from the group consisting of diacetyl, α-Tocopheryl acetate, 2,3-pentanedione, nicotine, carbonyls, benzene, toluene, metals, bacterial endotoxins, and fungal glucans.

In another aspect, described here is a method for treating an inflammatory disorder of lower airways in a human subject in need thereof, comprising administering an effective amount of anakinra directly to the lower airways in the human subject; wherein the effective amount of anakinra is from about 0.1 mg to about 200 mg per day; and wherein the inflammatory disorder is selected from the group consisting of a toxic-inhalation lung injury, pulmonary langerhans cell histiocytosis, non-cystic fibrosis bronchiectasis, diffuse panbronchiolitis, acute respiratory distress syndrome (ARDS), reactive airways dysfunction syndrome (RADS), bronchiolitis obliterans organizing pneumonia (BOOP), interstitial lung disease (ILD), idiopathic pulmonary fibrosis (IPF), restrictive allograft syndrome (RAS), pulmonary arterial hypertension (PAH), and pneumonitis.

In some embodiments, the toxic-inhalation lung injury is caused by inhalation of one or more chemical warfare agents. In some embodiments, the chemical warfare agent is selected from the group consisting of chlorine gas and sulfur mustard. In some embodiments, the toxic-inhalation lung injury is caused by inhalation of one or more environmental and/or industrial toxic agents. In some embodiments, the environmental and industrial toxic agents are selected from the group consisting of isocyanate, nitrogen oxide, morpholine, sulfuric acid, ammonia, phosgene, diacetyl, 2,3-pentanedione, 2,3-hexanedione, fly ash, fiberglass, silica, coal dust, asbestos, hydrogen cyanide, cadmium, acrolein, acetaldehyde, formaldehyde, aluminum, beryllium, iron, cotton, tin oxide, bauxite, mercury, sulfur dioxide, zinc chloride, polymer fumes, and metal fumes. In some embodiments, the toxic-inhalation lung injury is pneumoconiosis or bronchiolitis obliterans. In some embodiments, the toxic-inhalation lung injury is a vaping-associated lung injury. In some embodiments, the vaping-associated lung injury is caused by inhalation of one or more agents selected from the group consisting of diacetyl, α-Tocopheryl acetate, 2,3-pentanedione, nicotine, carbonyls, benzene, toluene, metals, bacterial endotoxins, and fungal glucans.

In some embodiments, the inflammatory disorder is selected from the group consisting of pulmonary langerhans cell histiocytosis, non-cystic fibrosis bronchiectasis, diffuse panbronchiolitis, acute respiratory distress syndrome (ARDS), reactive airways dysfunction syndrome (RADS), bronchiolitis obliterans organizing pneumonia (BOOP), restrictive allograft syndrome (RAS), pulmonary arterial hypertension (PAH), interstitial lung disease (ILD), idiopathic pulmonary fibrosis (IPF), and pneumonitis. In some embodiments, the inflammatory disorder is an inflammatory disorder of the lung. In some embodiments, anakinra is administered by a delivery device selected from the group consisting of a nebulizer, an inhaler, and a subminiature aerolizer. In some embodiments, the delivery device is a mesh nebulizer. In some embodiments, the mesh nebulizer is the Aerogen Solo. In some embodiments, the mesh nebulizer is the Aerogen Solo nebulizer.

Any one of the embodiments disclosed herein may be properly combined with any other embodiment disclosed herein. The combination of any one of the embodiments disclosed herein with any other embodiments disclosed herein is expressly contemplated. Specifically, the selection of one or more excipients from certain embodiments can be properly combined with the selection of one or more other particular excipients from other embodiments. Such combination can be made in any one or more embodiments of the application described herein or any formulation or composition described herein.

### BRIEF DESCRIPTION OF THE DRAWINGS

The following figures depict illustrative embodiments of the invention.

FIG. 1 illustrates a mechanism of action of an rhIL-1Ra that targets both innate and adaptive immune responses mediated by IL-1 signaling.

FIG. 2 shows a preparation procedure for ALTA-2530 solutions for nebulization.

FIG. 3 illustrates a tiered approach used for preformulation studies to create an ALTA-2530 solution for nebulization.

FIG. 4 is a graph showing viscosity results for various control and ALTA-2530 formulations across three different concentrations of anakinra (2.5 mg/mL, 20 mg/mL, and 50 mg/mL).

FIG. 5 is a plot showing X50 (or MMD, as an estimate for MMAD) for various control and ALTA-2530 formulations across three different concentrations of anakinra (2.5 mg/mL, 20 mg/mL, and 50 mg/mL).

FIG. 6 is a plot showing X50 (or MMD, as an estimate for MMAD) by viscosity for various control and ALTA-2530 formulations across three different concentrations of anakinra (2.5 mg/mL, 20 mg/mL, and 50 mg/mL).

FIG. 7 is a plot showing X50 (or MMD, as an estimate for MMAD) for various control and ALTA-2530 formulations.

FIG. 8 is a plot showing the liquid output rate (LOR, g/min) across nebulization cycle number for various control and ALTA-2530 formulations.

FIG. 9 is a plot showing total protein (μg) output across nebulization cycle number for various control and ALTA-2530 formulations.

FIGS. 10A-10F show plots of protein diameter (nm) by intensity (%) during testing using a surrogate method (shaking) at 10 minutes, 20 minutes, and 30 minutes across various control and ALTA-2530 formulations.

FIG. 11 is a plot showing concentration versus time profiles for rhIL-1Ra in ELF and serum following single doses to rat.

FIGS. 12A-12B are immunohistochemistry staining images of non-human primate lung for rh-IL-1Ra conducted using a commercially available anti-IL-1Ra antibody. FIG. 12A is an immunohistochemistry staining image of the lung of a non-human primate that received saline and FIG. 12B is an immunohistochemistry staining image of the lung of a non-human primate that received nebulized ALTA-2530 (0.86 mg/g).

FIGS. 13A-13F are immunohistochemistry staining images of rat lung tissue using a novel antibody that reduced non-specific staining. The images show rat lung after exposure to nebulized vehicle (FIG. 13A, 8 times magnified; FIG. 13B, 20 times magnified), after exposure to ALTA-2530 at a low dose (FIG. 13C, 8 times magnified; FIG. 13D, 20 times magnified), and after exposure to ALTA-2530 at a high dose (FIG. 13E, 8 times magnified; FIG. 13F, 20 times magnified).

FIGS. 14A-14B are graphs showing necrosis in the respiratory epithelium (FIG. 14A) and in the bronchial epithelium (FIG. 14B) for saline treated rats and for ALTA-2530 treated rats.

FIGS. 15A-15C are graphs showing concentration of ALTA-2530 by percentage maximum IL-6 release for human (FIG. 15A), NHP (FIG. 15B), and rat (FIG. 15C) blood.

FIG. 16 illustrates a phase 1 study protocol using single and/or multiple ascending doses (SAD/MAD) of ALTA-2530 in healthy volunteers and BOS patients.

FIG. 17 illustrates a phase 2b/3 study design of ALTA-2530 in BOS patients with 12-week proof of concept interim.

### DETAILED DESCRIPTION OF THE INVENTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The term “interleukin 1 receptor antagonist” or “IL-1Ra” refers to any peptide or protein that inhibits or blocks (either competitively or non-competitively) the activity of an interleukin-1 receptor (e.g., IL-1 type 1 receptor).

The term “anakinra” when used herein refers to a recombinant human interleukin 1 receptor antagonist (rhIL-1Ra) that is sequence identical to human IL-1Ra protein with an additional methionine amino acid on the N-terminus.

The term “ALTA-2530” when used herein refers to or describes any inhaled (e.g., nebulized) pharmaceutical composition comprising an IL-1Ra (e.g., anakinra, or a peptide IL-1R antagonist). In some embodiments, ALTA-2530 comprises a peptide IL-1Ra of about 50 amino acids in length or less.

The term “upper airways” or “upper respiratory tract” when used herein refers to or describes the central or conducting airways of the lungs including the passageways from flares or nostrils to the soft palate and includes the sinuses.

The term “lower airways” or “lower respiratory tract” when used herein refers to or describes the peripheral or alveolar region of the lungs below the larynx including the trachea and lungs.

The terms “treating,” “treatment,” and “therapy” as used herein refer to attempted reduction or amelioration of the progression, severity and/or duration of a disorder, or the attempted amelioration of one or more symptoms thereof resulting from the administration of one or more modalities (e.g., one or more therapeutic agents such as a compound or composition described herein).

As used herein, the terms “prevent,” “preventing” and “prevention” refer to the prevention or inhibiting of the recurrence, onset, or development of a disorder or a symptom thereof in a subject resulting from the administration of a therapy (e.g., a prophylactic or therapeutic agent), or the administration of a combination of therapies (e.g., a combination of prophylactic or therapeutic agents).

As used herein, “therapeutically effective amount” or “effective amount” refers to any amount that is necessary or sufficient for achieving or promoting a desired outcome. In some instances, an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, the terms “subject” and “patient” are used interchangeably herein. The terms “subject” and “subjects” refer to an animal, preferably a mammal including a nonprimate and a primate (e.g., a monkey such as a cynomolgus monkey, a chimpanzee, and a human), and more preferably a human. The term “animal” also includes, but is not limited to, companion animals such as cats and dogs; zoo animals; wild animals; farm or sport animals such as ruminants, non-ruminants, livestock and fowl (e.g., horses, cattle, sheep, pigs, turkeys, ducks, and chickens); and laboratory animals, such as rodents (e.g., mice, rats), rabbits; and guinea pigs, as well as animals that are cloned or modified, either genetically or otherwise (e.g., transgenic animals). In some embodiments, the term “subject” or “patient” refers to human.

As used herein, “a” or “an” means at least one, unless clearly indicated otherwise. The term “about,” unless otherwise indicated, refers to a value that is no more than 10% or 5% above or below the value being modified by the term. Thus, in some embodiments, the term “about 5% (w/w)” means a range of from 4.5% (w/w) to 5.5% (w/w). In other embodiments, the term “about 5% (w/w)” means a range of from 4.75% (w/w) to 5.25% (w/w).

As used herein, unless indicated otherwise, the terms “composition” and “composition of the invention”, are used interchangeably. Unless stated otherwise, the terms are meant to encompass, and are not limited to, pharmaceutical compositions and nutraceutical compositions containing drug substance (e.g., anakinra). The composition may also contain one or more “excipients” that are inactive ingredients or compounds devoid of pharmacological activity or other direct effect in the diagnosis, cure, mitigation, treatment, or prevention of disease or to affect the structure or any function of the human.

As used herein, the term “vehicle” refers to a diluent, placebo, adjuvant, excipient, carrier, or filler with which the compound or composition of the invention is stored, transported, and/or administered.

As used herein, the phrase “pharmaceutically acceptable salt” refers to pharmaceutically acceptable organic or inorganic salts of a compound of the invention.

As used herein, the term “pharmaceutically acceptable solvate” refers to an association of one or more solvent molecules and a compound of the invention. Examples of solvents that form pharmaceutically acceptable solvates include, but are not limited to water, saline, water-salt mixtures, isopropanol, ethanol, methanol, DMSO, ethyl acetate, acetic acid, polyethylene glycol and ethanolamine.

As used herein, the term “pharmaceutically acceptable” means approved by a regulatory agency of the Federal or a state government or listed in the U.S. Pharmacopeia or other generally recognized pharmacopeia for use in animals, and more particularly in humans.

Challenges Associated with Inhaled Delivery of Proteins

Currently, there is a paucity of FDA-approved intravenous (IV) or SC IL1-Ra (e.g., anakinra) for respiratory indications. This may result from the poor distribution of an IL1-Ra such as anakinra to the lung when delivered by the parenteral route and many other challenges of delivering a peptide or protein to the lungs. See Cawthorne et al. British J. Pharmacology 2010; Cawthorne et al. J. Pharmaceutical Sciences 1995. The liquid Kineret™ SC formulation has demonstrated cold-chain stability consistent with use as a pharmaceutical, however, as a protein biologic drug, rhIL-1Ra presents many formulation challenges for delivery via nebulization for oral inhalation. There are currently no FDA-approved IL1-Ra compositions for respiratory delivery (e.g., inhalation or nebulization).

These challenges include chemical and conformational stability during storage in the inhaled formulation where the protein must maintain integrity of size and proper conformation to retain potency. Another challenge is maintaining protein stability during nebulization including shear and temperature gradients. The Kineret™ formulation is prepared in a 10 mM citrate buffer. Although citrate is used in inhalation solutions, it has been shown to be a tussive agent at concentrations greater than ˜140 mM. Kineret™ formulation also includes citrate as a buffer. See Kaiser C, Knight A, Nordstrom D, Pettersson T, Fransson J, Florin-Robertsson E, Pilström B. Injection-site reactions upon Kineret (anakinra) administration: experiences and explanations. Rheumatol. Int. 2012 February; 32(2):295-9 (hereinafter “Kaiser 2012,” incorporated by reference in its entirety herein). Although citrate buffer is currently the only buffer on the FDA Inactive Ingredient (IIG) Database for respiratory delivery, citrate has been associated with pain on SC delivery and tissue irritation from mast cell degranulation, and thus may be poorly tolerated in the lungs. See Kaiser 2012. The Kineret™ formulation also contains higher than acceptable levels based on the current FDA IIG Database of polysorbate-80 for inhaled delivery, which could result in toxicity in treated patients. Additionally, there are compatibility challenges for inhaled delivery with hand-held, personal nebulizer devices, nebulizer devices used in hospital settings, and jet nebulizers. Compatibility challenges include adsorption, potential for leachables, large residual volumes, concentrating of the solution in the reservoir over the nebulization time, and degradation due to physical conditions including light exposure in the nebulizer. Furthermore, there are oxidative, shear, and temperature stresses on proteins as well during the nebulization process. See Hertel, S.; Pohl, T.; Friess, W.; Winter, G. Prediction of Protein Degradation during Vibrating Mesh Nebulization via a High Throughput Screening Method. Eur. J. Pharm. Biopharm. 2014, 87 (2), 386-394. In addition to oxidation, the heat or shear stress induced by these nebulizers may result in chemical instability of an IL-1Ra (e.g., anakinra) or cause the IL-1Ra (e.g., anakinra) to lose the proper confirmation for activity. There are further challenges with chemical and conformational stability during and after nebulization where the protein must maintain integrity of size and conformation to retain potency.

Additionally, it remains difficult to achieve and maintain nebulized particle sizes required for distribution through the upper and lower respiratory tract, where the lower (more distal) regions include bronchioles and alveolar sacs. This is important to ensure therapeutic exposure levels of drug in the diseased regions of the lung. The formulation of anakinra and the nebulizer selected need to generate appropriately sized anakinra particles (droplets) throughout the entire nebulization cycle and the anakinra droplets need to retain stability post nebulization and during inhalation, and adequately distribute throughout the intricate passages of the lung to reach bronchioles and alveoli to result in therapeutic effects. It is important to ensure consistent delivered dose generated from the nebulizer and consistent dose deposition of the delivered dose to the lung to maintain anakinra's efficacy. The formulation, e.g., excipients, needs to be carefully selected to ensure the protein is distributed to the target cell types associated with the respiratory disease indication. For example, for BOS, RAS, or Chronic Lung Allograft Dysfunction (CLAD) more generally, it is important that the formulation ensures the protein to enter bronchial epithelial cells and alveoli and preferably alveolar macrophages—depending on the specific indication.

The formulation also needs to be well tolerated in vivo. For example, the excipients selected ideally will not cause adverse events at clinical doses. In particular, there needs to be a safety margin (also referred to as a therapeutic window) between the highest non-adverse doses (usually determined in toxicology studies) and the clinically effective dose for treating patients. Otherwise, there needs to be an acceptable risk-benefit for administration to patients to tolerate the adverse effects. For instance, the acceptable adverse effects can include cough, which could occur with a dose lower than the therapeutically effective dose in the lung. The protein in the formulation also needs to be amenable to fill/finish procedures for the manufacture of clinical trial drug batches and approved product batches. Finally, while there have been FDA-approved small-molecule nebulization products, administration of proteins by nebulization is rare and to date only one nebulized protein drug, dornase alfa (Pulmozyme®), a recombinant human DNase used for the treatment of cystic fibrosis, has been approved by FDA.

### Novel Protein or Peptide Interleukin-1 Receptor Antagonist Compositions

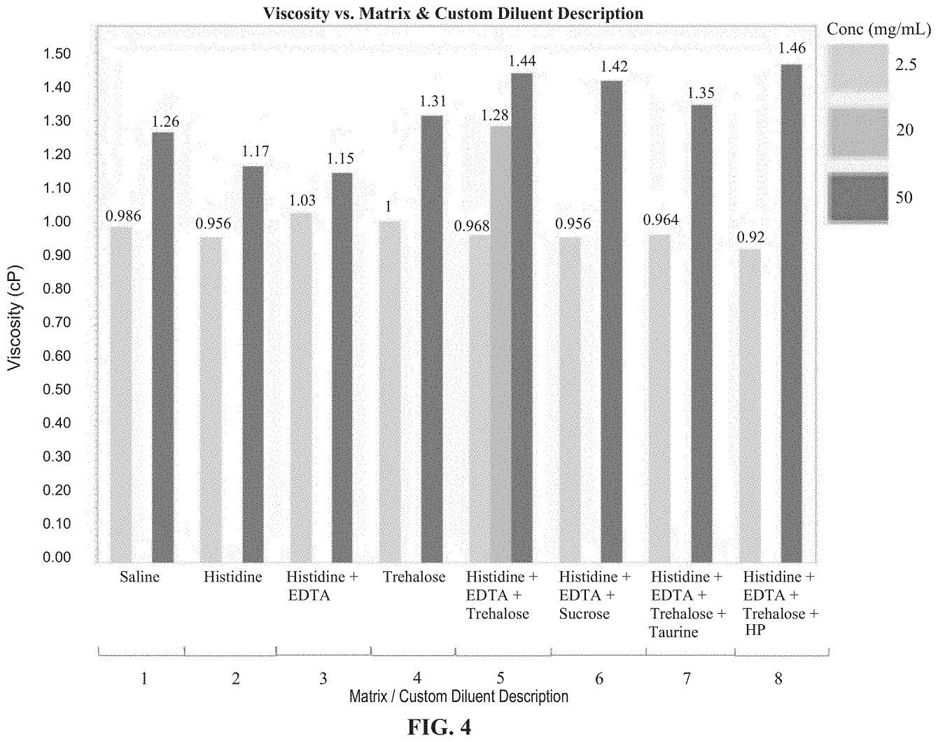

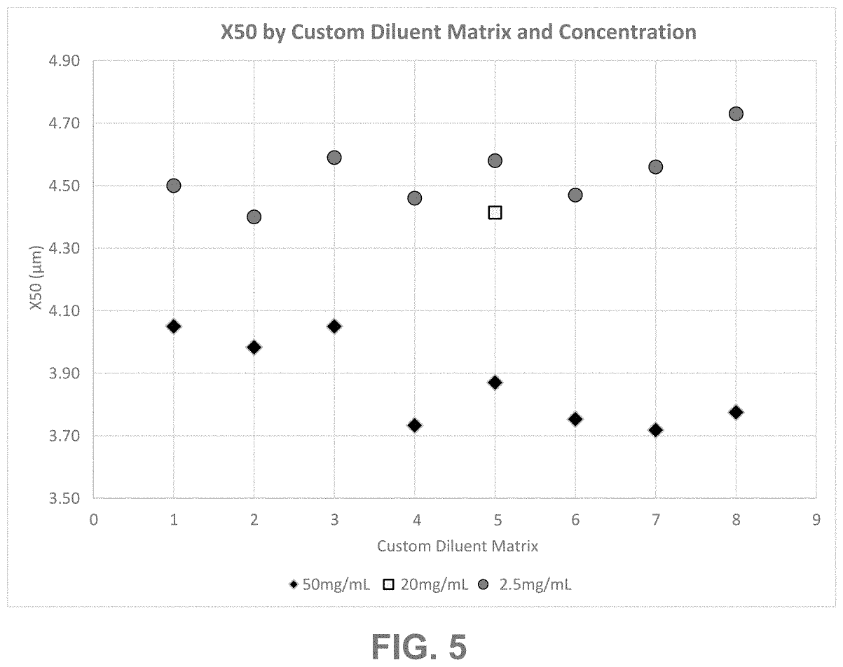

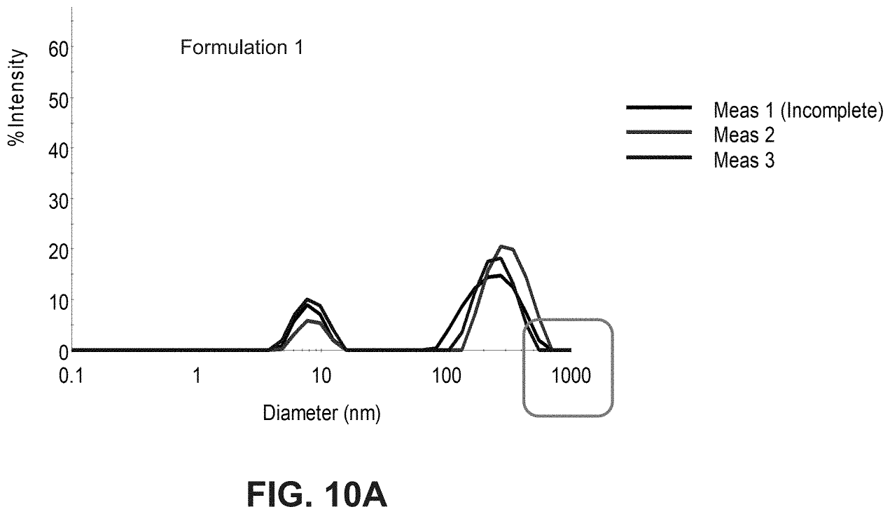

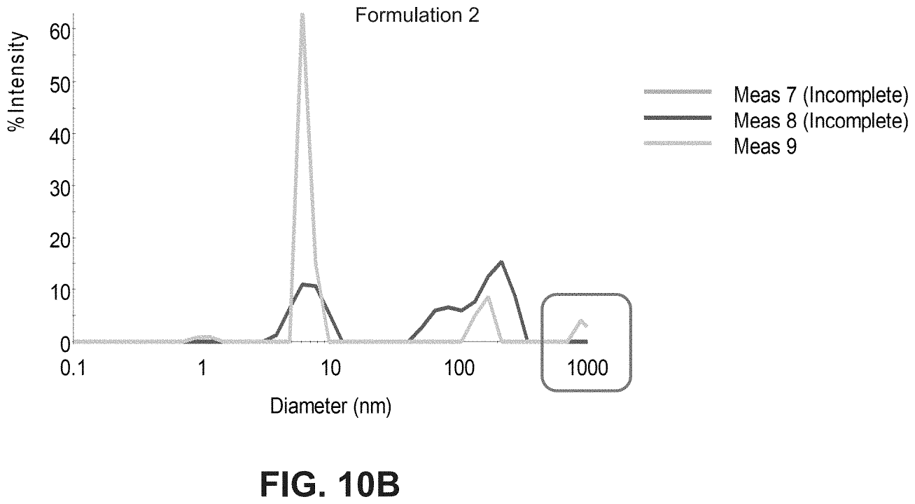

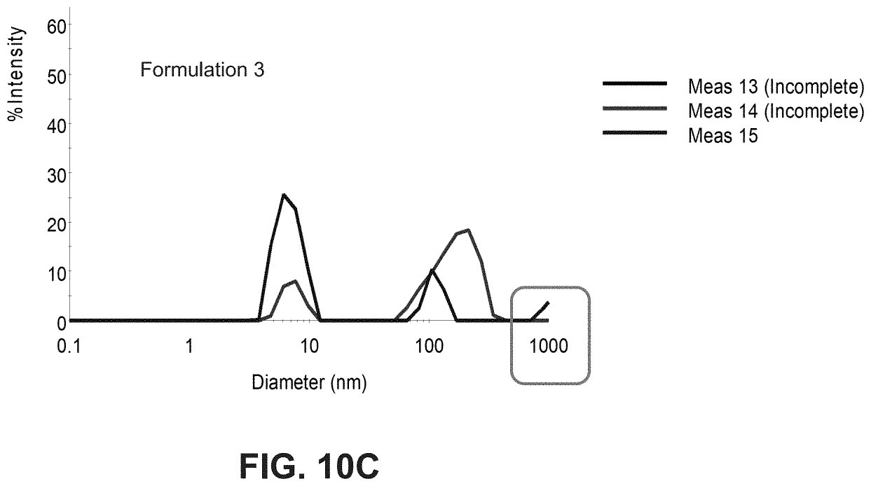

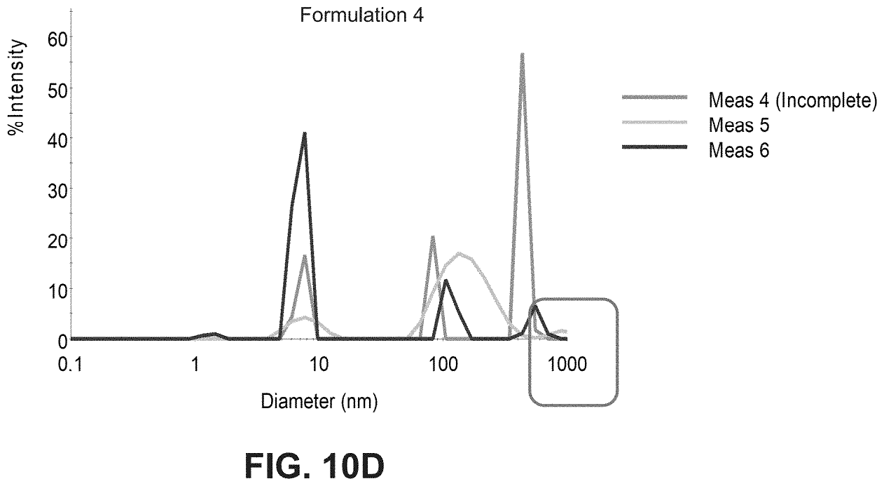

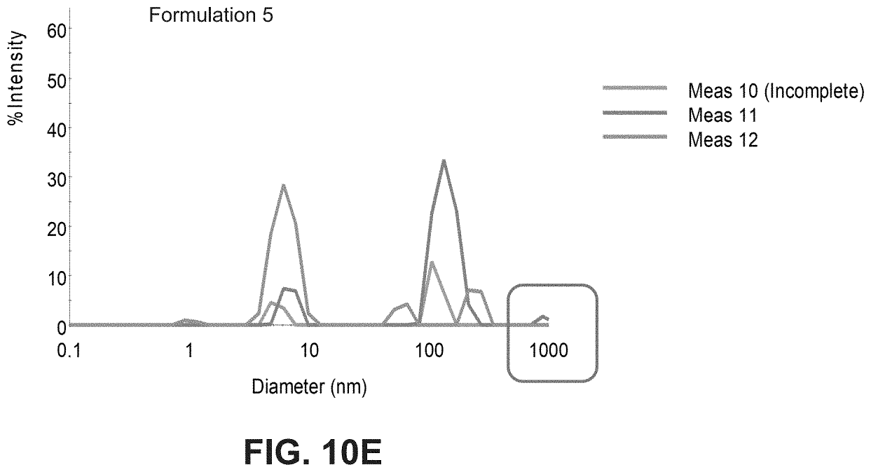

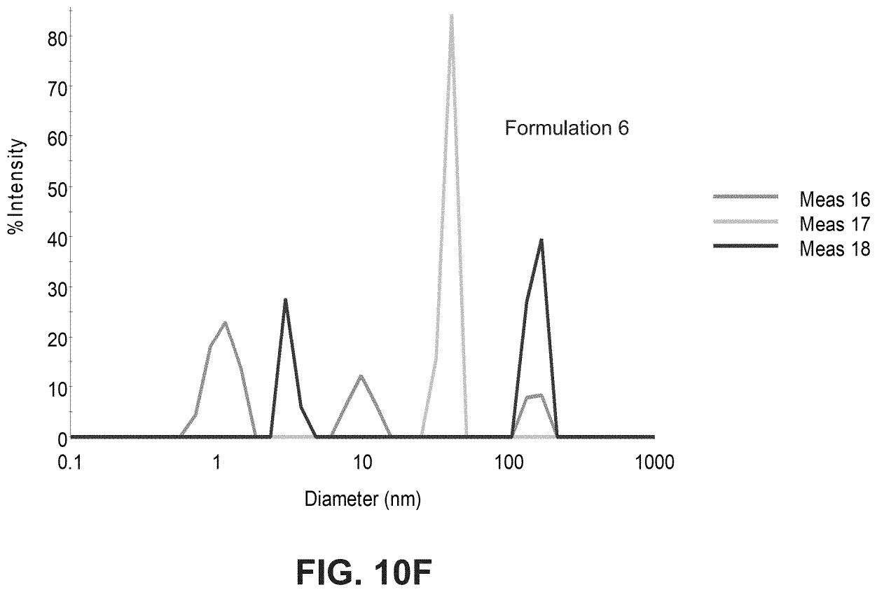

Described herein are novel inhaled/nebulized compositions of a protein or peptide interleukin-1 receptor antagonist (e.g., IL1-Ra; the composition of which is referred to as ALTA-2530) which have surprisingly been found to be advantageous for treating inflammatory disorders, including inflammatory disorders of the lower airways. In some embodiments, the protein or peptide IL-1Ra (e.g., anakinra) is included in an inhalation composition (e.g., a nebulized composition) with surprisingly advantageous properties such as: low protein aggregation (e.g., see Examples 8 and FIG. 10, Table 10), nebulized droplet sizes suitable for effective delivery to the lower respiratory tract (e.g., see Examples 6-7, Tables 7-9, and 11-12, and FIGS. 5-7), and protein stability (e.g., see Examples 6-7). In some embodiments, the protein or peptide IL-1Ra (e.g., anakinra) composition has these advantageous properties when combined with a suitable vibrating mesh nebulizer such as the PARI eFlow®, InnoSpire GO, or the Aerogen Solo (e.g., see Example 7). In some embodiments, the nebulizer is the PART eFlow® nebulizer (e.g., see Example 7).

In one aspect, a pharmaceutical composition is described (e.g., ALTA-2530), including a protein or peptide interleukin-1 receptor antagonist, a buffer, and optionally one or more additional components each selected from the group consisting of a stabilizer and a tonicity modifier. In some embodiments, the pharmaceutical composition is a liquid composition suitable for nebulization. In some embodiments, the buffer comprises an amino acid or phosphate. In some embodiments, the buffer comprises an amino acid. In some embodiments, the amino acid is selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, or a combination thereof. In some embodiments, the buffer comprises a positively charged amino acid. In some embodiments, the positively charged amino acid is histidine, arginine, or lysine. In some embodiments, the positively charged amino acid is histidine. In some embodiments, the pharmaceutical composition comprises a positively charged amino acid (e.g., histidine) in a concentration of between about 5 mM and 50 mM. In some embodiments, the concentration of histidine is about 10 mM. In some embodiments, the buffer comprises phosphate. In some embodiments, the pharmaceutical composition comprises phosphate in a concentration of about 5 mM and 50 mM. In some embodiments, the concentration of phosphate is about 10 mM.

In some embodiments, the pharmaceutical composition (e.g., ALTA-2530) includes a stabilizer. In some embodiments, the stabilizer is a non-reducing sugar. In some embodiments, the non-reducing sugar is trehalose, sucrose, glycerol, sorbitol, or a combination thereof. In some embodiments, the non-reducing sugar is trehalose. In some embodiments, the pharmaceutical composition comprises the non-reducing sugar (e.g., trehalose) in a concentration of between about 50 mM and 350 mM. In some embodiments, the pharmaceutical composition comprises the non-reducing sugar (e.g., trehalose) in a concentration of between about 50 mM and 100 mM. In some embodiments, the pharmaceutical composition comprises the non-reducing sugar (e.g., trehalose) in a concentration of between about 100 mM and 200 mM. In some embodiments, the pharmaceutical composition comprises the non-reducing sugar (e.g., trehalose) in a concentration of between about 100 mM and 300 mM. In some embodiments, the pharmaceutical composition comprises the non-reducing sugar (e.g., trehalose) in a concentration of between about 300 mM and 350 mM. In some embodiments, the pharmaceutical composition comprises the non-reducing sugar (e.g., trehalose) in a concentration of between about 100 mM and 150 mM. In some embodiments, the concentration of trehalose is about 115 mM. In some embodiments, the pharmaceutical composition includes ethylenediaminetetraacetic acid (EDTA) disodium as the stabilizer or as a stabilizer in addition to other added stabilizer(s). In some embodiments, the pharmaceutical composition comprises EDTA in a concentration of between about 0.05 mM and 1 mM. In some embodiments, the concentration of EDTA is about 0.53 mM. In some embodiments, the pharmaceutical composition includes taurine as the stabilizer or as a stabilizer in addition to other added stabilizer(s). In some embodiments, the pharmaceutical composition comprises taurine in a concentration of between about 50 mM and 150 mM. In some embodiments the taurine is in a concentration of about 80 mM. In some embodiments, the pharmaceutical composition comprises a tonicity modifier. In some embodiments, the tonicity modifier is sodium chloride. In some embodiments, the pharmaceutical composition comprises sodium chloride in a concentration of between about 10 mM and 160 mM. In some embodiments, the concentration of sodium chloride is about 90 mM.

In some embodiments, the pharmaceutical composition (e.g., ALTA-2530) comprises the interleukin-1 receptor antagonist (e.g., anakinra or another protein or peptide) in a concentration of between about 0.5 mg/mL and 50 mg/mL. In some embodiments, the pharmaceutical composition comprises the interleukin-1 receptor antagonist in a concentration of about 20 mg/mL. In some embodiments the pharmaceutical composition has a pH of between about 6 and about 8. In some embodiments the pharmaceutical composition has a pH of about 6.5. In some embodiments, the pharmaceutical composition is suitable for spray-drying.

In one embodiment, the pharmaceutical composition (e.g., ALTA-2530) is a liquid composition suitable for nebulization comprising an interleukin-1 receptor antagonist in a concentration of about 20 mg/mL, histidine in a concentration of about 10 mM, and trehalose in a concentration of about 115 mM. In some embodiments, the pharmaceutical composition further comprises sodium chloride in a concentration of about 20 mM, EDTA in a concentration of about 0.53 mM, polysorbate 80 in a concentration of about 0.05% (w/v) to about 0.1 (w/v) (e.g., 0.0125% or 0.035%), and the pharmaceutical composition has a pH of about 6.5.

In another embodiment, the pharmaceutical composition (e.g., ALTA-2530) is a liquid composition suitable for nebulization comprising an interleukin-1 receptor antagonist in a concentration of about 20 mg/mL, phosphate in a concentration of about 10 mM, and trehalose in a concentration of about 115 mM. In some embodiments, the pharmaceutical composition further comprises sodium chloride in a concentration of about 20 mM, EDTA in a concentration of about 0.53 mM, polysorbate 80 in a concentration of about 0.05% (w/v) to about 0.1% (w/v) (e.g., 0.0125% or 0.035%), and the pharmaceutical composition has a pH of about 6.5.

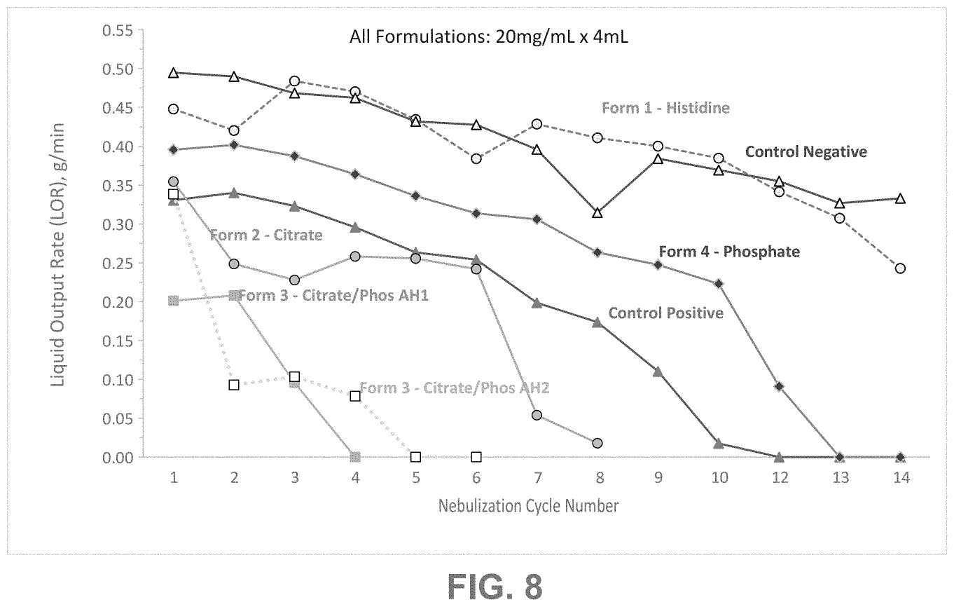

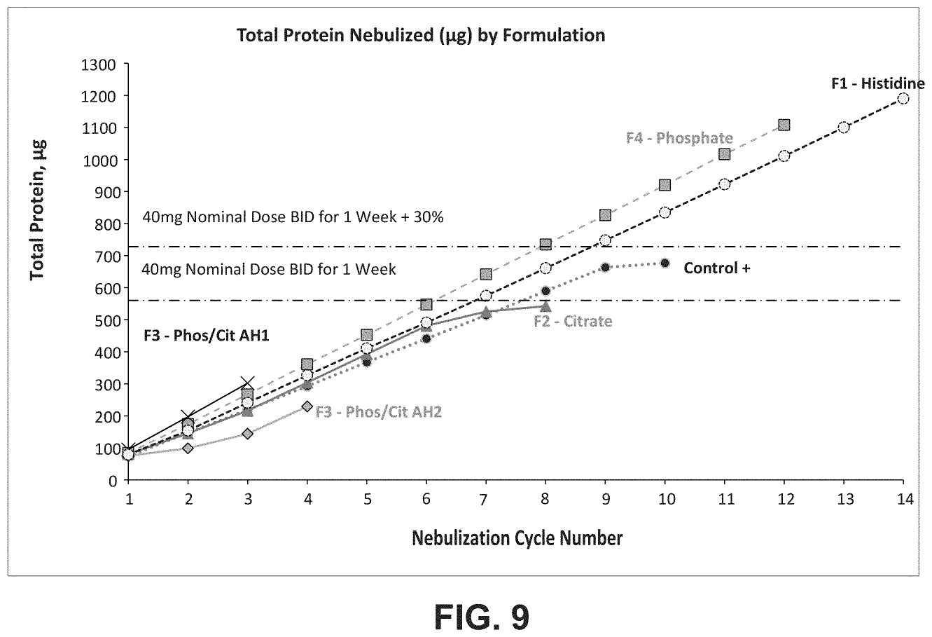

It has been surprisingly found that buffers comprising an amino acid (e.g., histidine; or others such as lysine, or arginine) or phosphate offer protection against protein aggregation in the protein or peptide interleukin-1 receptor antagonist composition (e.g., ability to achieve more nebulization cycles without aggregation) compared to multi-charged buffers such as citrate. For example, an ALTA-2530 formulation containing histidine as a buffer showed no time dependent increases in protein diameter suggesting that there was no time dependent aggregate formation when nebulized compared to a different formulation that instead contained a citrate buffer (e.g., see Example 8; compare formulation 1 (histidine) to formulation 2 (citrate) in FIGS. 8 and 9). Even formulations including both citrate and phosphate showed a drop-in liquid output rate with repeat nebulization suggesting clogging of the nebulizer potentially due to aggregation of the protein (e.g., see Example 8; compare formulation 1 (histidine) to formulation 3 (citrate and phosphate) in FIG. 8 and FIG. 9). In some embodiments, a formulation that uses histidine as a buffer outperforms a formulation that instead contains citrate as a buffer (see FIGS. 8 and 9). Additionally, in some embodiments, using a buffer comprising an amino acid or phosphate also allows for delivery of a greater amount of protein (IL1-Ra) without clogging (e.g., see Examples 7-8, and Tables 9 and 21, FIGS. 8 and 9). It has also been surprisingly found that buffers comprising an amino acid or phosphate increase buffer capacity and reduce shifts in pH with freeze-thaw cycles (e.g., see Example 8). It has been additionally surprisingly found that including polysorbate 80 in a concentration of about 0.01% (w/v) mitigates particulate formation of the protein or peptide interleukin-1 receptor antagonist when using a surrogate method (shaking plus heat, e.g., see Example 8 and FIG. 10).

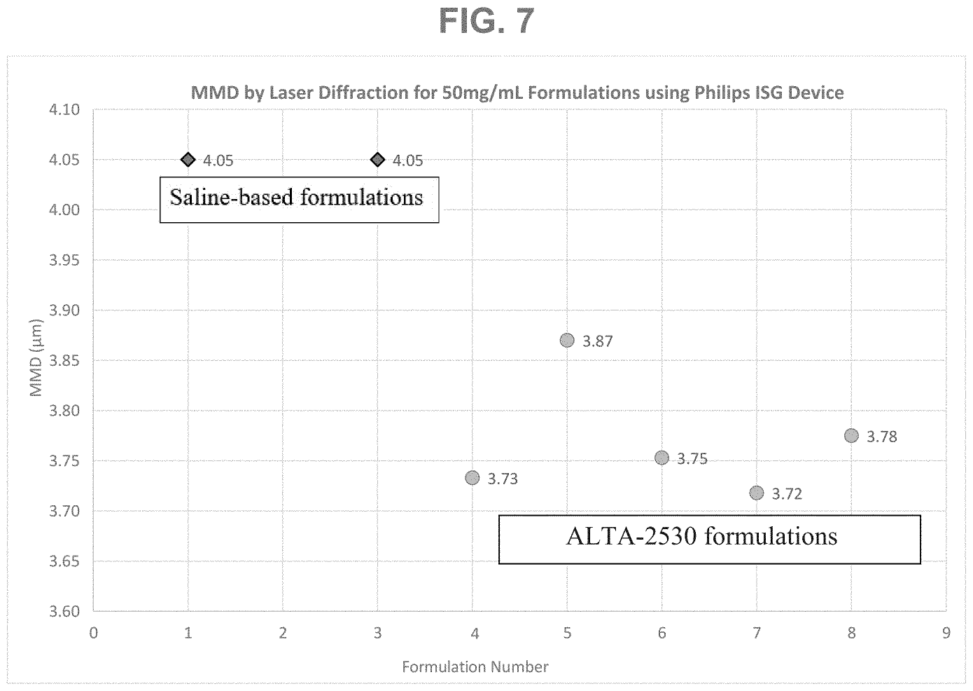

It has also been surprisingly found that in some embodiments, the protein or peptide interleukin-1 receptor antagonist (e.g., anakinra) compositions described herein have increased viscosity and produce smaller particle sizes of nebulized protein or peptide interleukin-1 receptor antagonist (e.g., anakinra) to a size more consistent with delivery to the distal regions of the lung versus dilution of the interleukin-1 receptor antagonist (e.g., anakinra) in saline (e.g., see Example 6 and FIGS. 4-6). In some embodiments, the droplet size of nebulized composition is between about 2.5 μm and 4 μm in diameter (e.g., Mass Median Aerodynamic Diameter (MMAD) or Mass Median Diameter (MMD or X50)). In some embodiments, the droplet size of nebulized composition is less than about 4 μm in diameter (e.g., MMAD, MMD) (e.g., see Example 7 and FIG. 7). In some embodiments, the droplet size of nebulized ALTA-2530 is about 3.5 μm in diameter (e.g., MMD, MMAD) (e.g., see Example 7 and, Table 9, FIG. 7). It has been surprisingly found that a combination of protein and sugars can be optimized to modulate viscosity to achieve MMAD or MMD values consistent with distal lung delivery. For example, it has been found that viscosity increases with protein concentration in ALTA-2530 and that non-reducing or high glass transition sugars (e.g., trehalose) in ALTA-2530 also increase viscosity (e.g., see Example 6 and FIGS. 4-6). In some embodiments, higher viscosity and higher protein concentration produces smaller aerodynamic particle sizes when ALTA-2530 is nebulized (e.g., see Example 6 and FIGS. 4-7). It has also been surprisingly found that non-reducing sugars or high glass transition sugars (e.g., trehalose) in ALTA-2530 also stabilize the IL-1Ra protein (e.g., see Example 6). In some embodiments, a high glass transition sugar refers to a sugar having a high glass transition temperature (Tg), e.g., more than 90° C. on an anhydrous basis. In some embodiments, Tg values 50° C. above the highest storage temperature can be considered high Tg sugars. For example, for dry powders stored at 25° C., a formulation can have a Tg of 75° C. or greater. See Ohtake, S.; Wang, Y. J. Trehalose: Current Use and Future Applications. J. Pharm. Sci. 2010, 1-34.

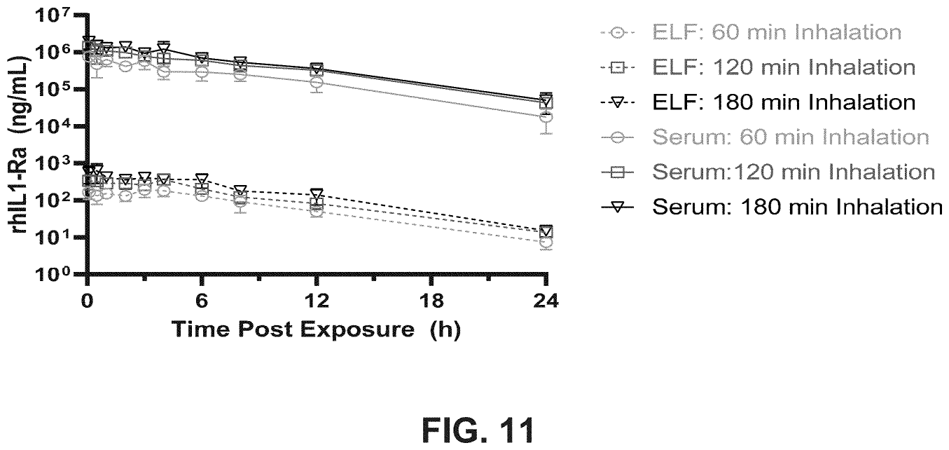

It has also been surprisingly found that ALTA-2530 can be delivered via a nebulizer with appropriate delivery efficiency, stability, and IL-1Ra potency and integrity after nebulization for delivery to the distal regions of lung (e.g., see Examples 7, 9-10, FIGS. 11-14). This is surprising due to the numerous challenges associated with nebulizing a large molecule (e.g., a protein or peptide such as an IL-1Ra) which is susceptible to loss of protein integrity due to denaturation when nebulized. In some embodiments, less than 1% loss of intact protein occurs following nebulization of ALTA-2530 (e.g., see Example 7 and Tables 13-14). In some embodiments, these advantageous results of nebulized ALTA-2530 can be achieved when ALTA-2530 is nebulized using a vibrating mesh nebulizer such as the PARI eFlow® nebulizer, the Philips InnoSpire GO nebulizer, or the Aerogen Solo nebulizer. In some embodiments, these advantageous results of nebulized ALTA-2530 can be achieved when ALTA-2530 is nebulized using a jet nebulizer such as the AeroEclipse II nebulizer, the PARI LC Plus®, or the PARI LC Sprint®. In some embodiments, ALTA-2530 can be nebulized without clogging the nebulizer device with repeat use (e.g., see Example 7). In some embodiments, delivering ALTA-2530 using a mesh nebulizer surprisingly increases the amount of protein delivered compared to using jet nebulizers and results in more consistent and higher delivered dose. (e.g., see Example 8 and FIG. 9 showing high protein output for formulation 1, which contained histidine, at the end of 14 cycles using the modified PARI eFlow® nebulizer. In some embodiments, delivering ALTA-2530 using a mesh nebulizer is gentler on the protein than using a jet nebulizer (single pass versus solution recirculation) and can therefore reduce protein aggregation.

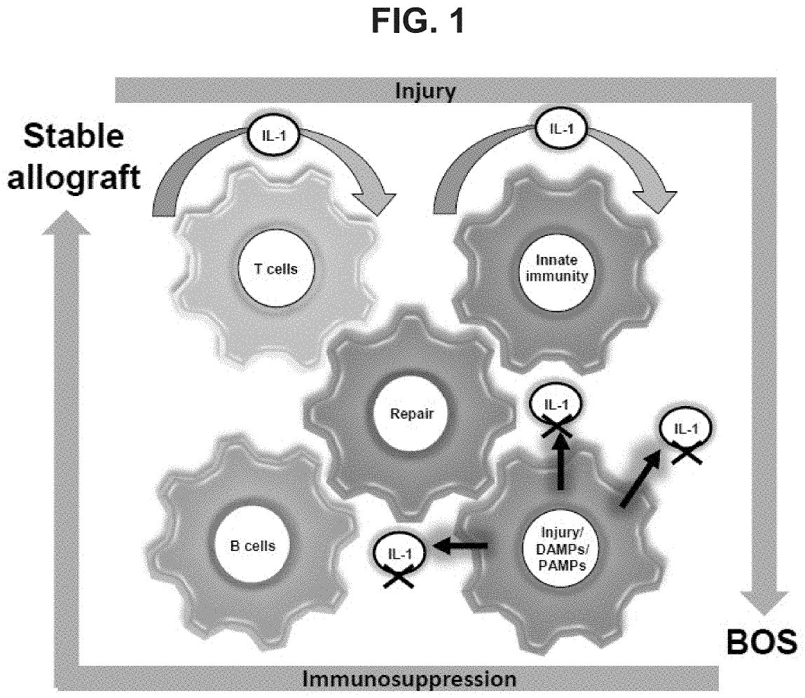

In certain embodiments, a novel pharmaceutical composition comprising an IL1-Ra is described. In some embodiments, the pharmaceutical composition is delivered directly to target tissue via inhalation which solves for impaired perfusion in post-LT patients and limits systemic side effects. In some embodiments, the pharmaceutical composition has a novel mechanism of action which targets innate immune response in BOS (e.g., see FIG. 1). In some embodiments, the pharmaceutical composition exhibits a strong safety profile based on successful early non-clinical experience (e.g., see Examples 9-10 and 12).

In certain embodiments, a pharmaceutical composition is described, including an interleukin-1 receptor antagonist and one or more additional components each selected from the group consisting of a buffer, a stabilizer, and a tonicity modifier.

In some embodiments, the interleukin-1 receptor antagonist is anakinra. Other interleukin-1 receptor antagonists are contemplated. In certain embodiments, the pharmaceutical compositions described herein are examples for formulations of anakinra for nebulized delivery.

In certain embodiments, the buffer comprises a positively charged amino acid.

In certain embodiments, the buffer is selected from the group consisting of citrate, phosphate, succinate, histidine, lysine, arginine, glutamate, pyrophosphate, 4-(2-hydroxyethyl)-1-piperazineethanesulfonic acid (HEPES), and a combination thereof. In some embodiments, the pharmaceutical composition is a liquid composition comprising citrate in a concertation of between about 0.5 mM and 20 mM.

In some embodiments, the concentration of citrate is about 20 mM. In some embodiments, the pharmaceutical composition is a liquid composition comprising phosphate in a concentration of between about 1 mM and 50 mM, or about 10 mM.

In some embodiments, the pharmaceutical composition is a liquid composition comprising histidine in a concentration of between about 5 mM and 50 mM or about 10 mM.

In some embodiments, the pharmaceutical composition is a liquid composition comprising glutamate in a concentration of between about 1 mM and 50 mM. In some embodiments, the pharmaceutical composition is a liquid composition comprising pyrophosphate in a concentration of between about 1 mM and 50 mM.

In some embodiments, the pharmaceutical composition is a liquid composition comprising 4-(2-hydroxyethyl)-1-piperazineethanesulfonic acid (HEPES) in a concentration of between about 10 mM and 50 mM or about 10 mM.

In some embodiments, the stabilizer is selected from the group consisting of a surfactant, a chelating agent, a sugar, and a combination thereof. In some embodiments, the surfactant is selected from the group consisting of polysorbate 80, polysorbate 20, polyoxyethylene (23) lauryl ether (Brij™ 35), sorbitan trioleate (Span™ 85), and a combination thereof.

In some embodiments, the sugar is a non-reducing sugar, and wherein the non-reducing sugar is selected from the group consisting of trehalose, sucrose, glycerol, sorbitol, and a combination thereof. In some embodiments, the pharmaceutical composition is a liquid composition comprising trehalose in a concentration of between about 100 nM and 350 nM. In some embodiments, the concentration of trehalose is about 115 nM.

In some embodiments, the pharmaceutical composition is a liquid composition comprising polysorbate 80 in a concentration of between about 0.01% and 1% (w/v) or about 0.05% to 0.1% (w/v) (e.g., 0.0125 to 0.035% w/v).

In some embodiments, the pharmaceutical composition is a liquid composition comprising polysorbate 20 in a concentration of between about 0.00001% and 1% (w/v), or between about 0.00001% and 0.01% (w/v). In any one of the embodiments described herein, the pharmaceutical composition is a liquid composition comprising polysorbate 20 in a concentration of about 0.00001% (w/v), 0.0001% (w/v), or 0.001% (w/v).

In some embodiments, the pharmaceutical composition is a liquid composition comprising polyoxyethylene (23) lauryl ether (Brij™ 35) in a concentration of between about 0.00001% and 0.01% (w/v). In some embodiments, the pharmaceutical composition is a liquid composition comprising sorbitan trioleate (Span™ 85) in a concentration of between about 0.1% and 5.0% (w/v), about 0.8 (w/v), 0.85 (w/v), or 0.86% (w/v).

In some embodiments, the chelating agent is ethylenediaminetetraacetic acid (EDTA) disodium. In some embodiments, the pharmaceutical composition is a liquid composition comprising ethylenediaminetetraacetic acid (EDTA) disodium in a concentration of between about 0.05 mM and 1 mM or about 0.53 mM.

In some embodiments, the sugar is selected from the group consisting of trehalose, sucrose, glycerol, sorbitol, and a combination thereof. In some embodiments, the pharmaceutical composition is a liquid composition, and the concentration of the sugar is between about 1% (w/v) and 5% (w/v), between about 5% (w/v) and 10% (w/v), between about 10% (w/v) and 15% (w/v), between about 10% (w/v) and 20% (w/v), between about 20% (w/v) and 30% (w/v), or between about 30% (w/v) and 40% (w/v). In some embodiments, the pharmaceutical composition is a liquid composition, and the concentration of the sugar is about 4% (w/v). In some embodiments, the pharmaceutical composition is a liquid composition, and the concentration of the sugar is about 12% (w/v). In some embodiments, the pharmaceutical composition is a liquid composition, and the concentration of the sugar is greater than about 40% (w/v).

In some embodiments, the tonicity modifier is selected from the group consisting of sodium chloride, mannitol, taurine, hydroxyproline, proline, and a combination thereof. In some embodiments, the pharmaceutical composition is a liquid composition comprising sodium chloride in a concentration of between about 10 mM and 160 mM or about 90 mM.

In some embodiments, the pharmaceutical composition is a liquid composition comprising mannitol in a concentration of between about 5 mg/mL and 50 mg/mL or about 10 mg/mL.

In some embodiments, the pharmaceutical composition is a liquid composition comprising taurine in a concentration of between about 5 mg/mL and 50 mg/mL or about 10 mg/mL.

In some embodiments, the pharmaceutical composition is a liquid composition comprising hydroxyproline in a concentration of between about 0.2 mg/mL and 50 mg/mL or about 3 mg/mL.

In some embodiments, the additional components comprise citrate, ethylenediaminetetraacetic acid (EDTA) disodium, polysorbate 80, and sodium chloride. In some embodiments, the additional components comprise phosphate, ethylenediaminetetraacetic acid (EDTA) disodium, polysorbate 80, and sodium chloride.

In some embodiments, the additional components comprise phosphate, mannitol, ethylenediaminetetraacetic acid (EDTA) disodium, and sodium chloride. In some embodiments, the additional components comprise phosphate, mannitol, ethylenediaminetetraacetic acid (EDTA) disodium, polysorbate 80, and sodium chloride.

In some embodiments, the additional components comprise phosphate, mannitol, ethylenediaminetetraacetic acid (EDTA) disodium, polysorbate 20, and sodium chloride. In some embodiments, the additional components comprise phosphate, mannitol, ethylenediaminetetraacetic acid (EDTA) disodium, sorbitan trioleate (Span™ 85), and sodium chloride.

In some embodiments, the additional components comprise phosphate, trehalose, ethylenediaminetetraacetic acid (EDTA) disodium, polysorbate 80, and sodium chloride. In some embodiments, the additional components comprise phosphate, sucrose, ethylenediaminetetraacetic acid (EDTA) disodium, polysorbate 80, and sodium chloride.

In some embodiments, the additional components comprise phosphate, ethylenediaminetetraacetic acid (EDTA) disodium, a tonicity modifier, and sodium chloride. In some embodiments, the additional components comprise phosphate, mannitol, ethylenediaminetetraacetic acid (EDTA) disodium, polysorbate 80, a tonicity modifier, and sodium chloride.

In some embodiments, the additional components comprise phosphate, trehalose, ethylenediaminetetraacetic acid (EDTA) disodium, polysorbate 20, a tonicity modifier, and sodium chloride. In some embodiments, the additional components comprise phosphate, sucrose, ethylenediaminetetraacetic acid (EDTA) disodium, sorbitan trioleate (Span™ 85), a tonicity modifier, and sodium chloride.

In some embodiments, the additional components comprise phosphate, a tonicity modifier, and sodium chloride. In some embodiments, the tonicity modifier is selected from the group consisting of taurine, hydroxyproline, and a combination thereof.

In some embodiments, the additional components comprise citrate, phosphate, ethylenediaminetetraacetic acid (EDTA) disodium, polysorbate 80, and sodium chloride. In some embodiments, the additional components comprise glutamate, ethylenediaminetetraacetic acid (EDTA) disodium, polysorbate 80, and sodium chloride.

In some embodiments, the additional components comprise citrate, trehalose, ethylenediaminetetraacetic acid (EDTA) disodium, polysorbate 80, and sodium chloride.

In some embodiments, the additional components comprise glutamate, mannitol, ethylenediaminetetraacetic acid (EDTA) disodium, polysorbate 80, and sodium chloride. In some embodiments, the additional components comprise phosphate, mannitol, and sodium chloride.

In some embodiments, the additional components comprise histidine, trehalose, sodium chloride, and ethylenediaminetetraacetic acid (EDTA) disodium.

In some embodiments, the additional components comprise phosphate, trehalose, sodium chloride, and ethylenediaminetetraacetic acid (EDTA) disodium.

In some embodiments, the pH of the liquid composition is about 6.5.

In some embodiments, the pharmaceutical composition is a liquid composition. In some embodiments, the pharmaceutical composition is a solid composition.

In some embodiments, the solid composition comprises dry powder. In some embodiments, the solid composition comprises dry powder including a non-reducing sugar such as trehalose. In some embodiments, the solid composition is a lyophilisate. In some embodiments, the pharmaceutical composition is reconstituted from a lyophilized powder.

In some embodiments, the pharmaceutical composition is a liquid composition comprising the interleukin-1 receptor antagonist in a concentration of between about 1 mg/mL and 50 mg/mL.

In some embodiments, the concentration of the interleukin-1 receptor antagonist is about 5 mg/mL.

In some embodiments, the concentration of the interleukin-1 receptor antagonist is about 20 mg/mL. In some embodiments, the concentration of the interleukin-1 receptor antagonist is about 10 mg/mL. In some embodiments, the concentration of the interleukin-1 receptor antagonist is about 30 mg/mL. In some embodiments, the concentration of the interleukin-1 receptor antagonist is about 10-30 mg/mL.

### Kits

In another aspect, a kit is described, including a pharmaceutical composition according to any one of the embodiments disclosed herein and a delivery device suitable for direct administration of the pharmaceutical composition to the respiratory tract of a patient.

In another aspect, a kit is disclosed, including a pharmaceutical composition according to any one of embodiments described herein and a delivery device suitable for direct administration of the pharmaceutical composition to the respiratory tract of a patient.

In some embodiments, the respiratory tract comprises the lower or upper airways.

In some embodiments, the delivery device is configured to deliver an effective amount of the pharmaceutical composition via inhalation. In some embodiments, the delivery device is configured to deliver an effective amount of the pharmaceutical composition via direct instillation.

In some embodiments, the delivery device is selected from the group consisting of a nebulizer, an inhaler, and an aerolizer. In some embodiments, the delivery device is selected from the group consisting of a jet nebulizer, an ultrasonic nebulizer, a metered dose inhaler, and a dry powder inhaler. In some embodiments, the nebulizer is a vibrating mesh nebulizer. In some embodiments, the nebulizer is a jet nebulizer. In some embodiments, the nebulizer is selected from the group consisting of the Philips InnoSpire GO, a PARI nebulizer (e.g., the PARI eFlow®, the PARI LC Plus®, or the PART LC Sprint®), the Aerogen Solo, and the AeroEclipse II.

In some embodiments, the pharmaceutical composition is a solution for nebulization delivered using a Philips InnoSpire GO vibrating mesh (VM) nebulizer. In some embodiments, the pharmaceutical composition is a solution for nebulization that will be delivered using a preclinical nebulizer. In some embodiments, the pharmaceutical composition is an extemporaneously prepared solution formulation for nebulization that can be produced at the preclinical and clinical study sites and is stable for nebulization over the dosing period and a minimum in-use period of 24 hours. In some embodiments, the pharmaceutical composition is a solution for nebulization stored at refrigeration temperatures. In some embodiments, the pharmaceutical composition developed for Good Laboratory Practice (GLP) toxicology and Good Manufacturing Practice (GMP) clinical studies will preferably be the same or comparable to avoid any bridging studies (e.g., excipients will not differ, and ratios will not exceed GLP qualification levels). In some embodiments, the pharmaceutical composition's impurity profiles of the nebulized GMP clinical formulation will be similar to and will not exceed the impurity limits qualified in the GLP preclinical studies. In some embodiments, the pharmaceutical composition is a clinical formulation solution having concentration(s) suited to deliver 5-80 mg from the VM nebulizer (expressed as drug charge (nominal dose) to nebulizer) in less than 15 minutes, ideally less than 10 minutes, optimally less than 5 minutes, or within 2-3 minutes, using, for example, the PARI eFlow® nebulizer or the Philips InnoSpire GO nebulizer. In some embodiments, the pharmaceutical composition is reproducibly delivered, and pulmonary lung dose supports the clinical programs as demonstrated by chemical and aerosol performance stability over the in-use period and anticipated dosing duration. In some embodiments, the pharmaceutical composition has stability similar to or greater than the tolerability thresholds qualified in freeze/thaw studies. In some embodiments, the pharmaceutical composition is stable based on stress stability studies (e.g., in vitro or CMC studies). In some embodiments, the pharmaceutical composition meets purity standards based on filter compatibility studies. In some embodiments, the pharmaceutical composition does not exceed loss of content thresholds based on filter compatibility studies. In some embodiments, the pharmaceutical composition's stability of the nebulized GMP clinical formulation is similar to or greater than the stability thresholds qualified in CMC product development studies. In some embodiments, the pharmaceutical composition's in-use period of the nebulized GMP clinical formulation is similar to the in-use period qualified in CMC product development studies. In some embodiments, the pharmaceutical composition's storage conditions of the nebulized GMP clinical formulation are similar to the storage conditions qualified in CMC product development studies. In some embodiments, the pharmaceutical composition's pH, osmolality, and appearance are similar to measures qualified in CMC product development studies. In some embodiments, a protein concentration of the pharmaceutical composition is similar to a concentration qualified in CMC product development studies. In some embodiments, purity of the pharmaceutical composition is similar to measures qualified in RP-HPLC, SE-HPLC, reduced and non-reduced CE-SDS, and IEX-HPLC CMC product development studies. In some embodiments, the levels of foreign and particulate matter, and subvisible particles in the pharmaceutical composition are similar to levels qualified in CMC product development studies. In some embodiments, the levels of foreign and particulate matter, and subvisible particles in the pharmaceutical composition are similar to levels qualified in CMC product development studies. In some embodiments, the pharmaceutical composition's aerosol particle size distribution is measured by NGI (next generation impactor) and in accordance with US Pharmacopeial Convention (e.g., USP Chapter 601 and 1601). In some embodiments, the pharmaceutical composition's delivered dose using breath simulator is measured by the dose listed in USP 1601 and USP 601 over the entire duration of dosing. In some embodiments, the pharmaceutical composition's potency will be similar to potency qualified in in vitro cell-based bioassay studies. In some embodiments, the pharmaceutical composition's measure of viscosity, surface tension, formulation density, droplet size and distribution (e.g., as measured by Malvern Spraytec or NGI or equivalent), dynamic light scattering (DLS), and turbidity will be similar to measures qualified in CMC development studies. In some embodiments, droplet size distributions for ALTA-2530 formulations obtained with Aerogen Solo used for nonclinical studies and in vitro studies and droplet size distributions obtained with other nebulizers achieve comparable distribution in pulmonary tissue.

In some embodiments, the pharmaceutical composition is a liquid composition, and the delivery device is configured to deliver the liquid composition. In some embodiments, the pH of the liquid composition is between about 5 and 8.

In some embodiments, the osmolality of the liquid composition is between about 200 mOsm/kg and 500 mOsm/kg. In some embodiments, the osmolality is about 300 mOsm/kg.

In some embodiments, the aerodynamic droplet size of the liquid composition produced by the delivery device is between about 0.5 μm and 10 μm in diameter. In some embodiments, the MMAD of the liquid composition produced by the delivery device is between about 2.5 μm and 4 μm in diameter. In some embodiments, the aerodynamic droplet size of the liquid composition produced by the delivery device is about 3.5 μm in diameter. In some embodiments, the aerodynamic droplet size of the liquid composition produced by the delivery device is suitable for preferentially targeting the lower airways.

In some embodiments, the aerodynamic droplet size of the liquid composition produced by the delivery device is between about 5 μm and 50 μm in diameter. In some embodiments, the aerodynamic droplet size of the liquid composition produced by the delivery device is suitable for preferentially targeting the upper airways. In some embodiments, the conductivity of the liquid composition is less than 2.5 μS/cm.

In some embodiments, the liquid formulation disclosed here is amenable to drying, e.g., spray drying, or lyophilization to afford a solid formulation or lyophilizate. In some embodiments, the pharmaceutical composition is a solid composition and the delivery device is configured to deliver the solid composition. In some embodiments, the solid composition comprises particles having a MMAD or MMD between about 0.1 μm and 20 μm. In some embodiments, the MMAD of MMD of the particles is less than about 5 μm. In some embodiments, the MMAD or MMD of the particles is less than about 3.5 μm.

In some embodiments, the solid composition comprises particles having a MMAD or MMD between about 1 μm and 10 μm.

In some embodiments, the solid composition has a tap density of less than about 1 g/cm³. In some embodiments, the solid composition has a rugosity between about 1 and 6.

In some embodiments, the solid composition comprises porous particles. In some embodiments, the solid composition comprises swellable particles.

In some embodiments, the porous particles comprise biodegradable polymers. In some embodiments, the solid composition further comprises a salt of a fatty acid or a derivative thereof.

In some embodiments, the salt is selected from the group consisting of magnesium stearate, sodium stearyl fumarate, sodium stearyl lactylate, sodium lauryl sulfate, magnesium lauryl sulfate, and a combination thereof. In some embodiments, the solid composition comprises particles having uniform particle size distribution.

In some embodiments, the solid composition comprises particles having nonuniform particle size distribution. In some embodiments, the solid composition comprises particles having bimodal particle size distribution.

In some embodiments, the percent mass of the interleukin-1 antagonist in the solid composition is between about 1% and 40%, 40% and 70%, or more than 70%.

In some embodiments, the solid composition comprises a plurality of particles enclosed in a plurality of receptacles. In some embodiments, the receptacles are selected from the group consisting of capsules, blisters, and film covered containers. In some embodiments, the delivery device is suitable for direct administration of the pharmaceutical composition to bronchioles. In some embodiments, the delivery device is suitable for direct administration of the pharmaceutical composition to alveolar tissue.

### Method of Treating Inflammatory Disorders

In one aspect, a method for treating an inflammatory disorder is described. In certain embodiments, the method includes administering an effective amount of anakinra directly to the lower airways in the human subject. Anakinra is a recombinant and modified version of the human interleukin-1 receptor antagonist protein (IL-1Ra). In some embodiments, the method includes administering an effective amount of protein or peptide interleukin-1 receptor antagonist inhalation formation, e.g., ALTA-2530, which is a novel formulation of an IL-1Ra as described herein. In some embodiments, ALTA-2530 is an inhaled formulation of anakinra.

IL-1 type 1 receptor activity (agonism) induces a myriad of secondary inflammatory mediators, including prostaglandins, cytokines, and chemokines. Anakinra blocks the biologic activity of the endogenous IL-1α and IL-1β cytokines, which are part of the Interleukin-1 family (“IL-1”), by competitively inhibiting IL-1α and IL-1β's binding to the interleukin-1 type I receptor (IL-1RI).

It has been surprisingly found that anakinra can be directly administered to the lower airways, e.g., the lung, in the human subject having the inflammatory disorder disclosed herein to effectively treat the inflammatory disorder. In some embodiments, the inflammatory disorder disclosed herein is associated with or at least in part associated with the lower airways. Without wishing to be bound by any specific theory, it is believed that in the human subject having the inflammatory disorder disclosed herein, IL-1α and IL-1β bind to the interleukin-1 type I receptor which triggers inflammation and that anakinra blocks the activities of these IL-1 cytokines local to the lower airways, thereby effectively treating inflammation and the inflammatory disorder.

Thus, in one aspect, a method for treating an inflammatory disorder of lower airways in a human subject in need thereof is described, including administering an effective amount of anakinra directly to the lower airways in the human subject; and where the inflammatory disorder is selected from the group consisting of a bronchiolitis obliterans syndrome (BOS), chronic obstructive pulmonary disease (COPD), toxic-inhalation lung injury, pulmonary langerhans cell histiocytosis, non-cystic fibrosis bronchiectasis, diffuse panbronchiolitis, acute respiratory distress syndrome (ARDS), reactive airways dysfunction syndrome (RADS), bronchiolitis obliterans organizing pneumonia (BOOP), restrictive allograft syndrome (RAS), pulmonary arterial hypertension (PAH), interstitial lung disease (ILD), idiopathic pulmonary fibrosis (IPF), and pneumonitis. In some embodiments, the inflammatory disorder is an inflammatory disorder of the lung. In some embodiments, the inflammatory disorder is pneumonitis or pneumoconiosis. In some embodiments, the effective amount of anakinra or ALTA-2530 is from about 0.1 mg to about 200 mg per day.

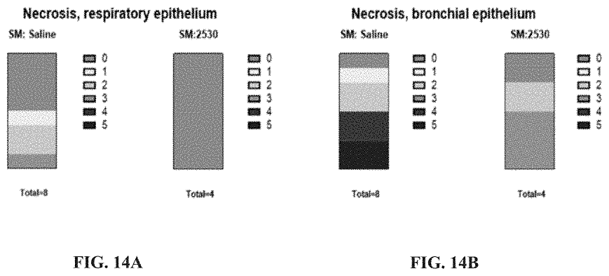

As used herein, a toxic-inhalation lung injury includes any injury (e.g., inflammation or damage) to the lung as a result of inhalation of one or more foreign and/or toxic agents. In some embodiments, the subject having a toxic-inhalation lung injury suffers from inflammation mediated by IL-1α and IL-1β and ALTA-2530 blocks the activities of these cytokines local to the upper and lower airways and thereby effectively treats inflammation and the toxic-inhalation lung injury. In some embodiments, ALTA-2530 reduces necrosis in upper and lower respiratory tract (e.g., see FIGS. 14A-14B).

In some embodiments, the toxic-inhalation lung injury is caused by inhalation of one or more chemical warfare agents. Non-limiting examples of the chemical warfare agents include chlorine gas and sulfur mustard. There is no approved treatment for sulfur mustard inhalation injury. Mustard gas causes acute effects such as bleeding/blistering in the lungs, damage to mucous membranes, and pulmonary edema, and chronic effects such as parenchymal fibrosis and BOS. Other examples of the chemical warfare agents known in the art are contemplated. Chlorine and Sulfur Mustard gases cause acute effects such as cell death, acute pulmonary edema, airway hyperresponsiveness, and inflammasome activation, and chronic effects such as airway hyperresponsiveness and BOS. In some embodiments, the toxic-inhalation lung injury is chlorine-induced BOS. In some embodiments, the toxic-inhalation lung injury is sulfur mustard-induced BOS.

In other embodiments, the toxic-inhalation lung injury is caused by inhalation of one or more environmental and/or industrial toxic agents. Various toxic agents exist in the environmental (natural or artificial) and industrial setting. A human subject may come into contact with and inhale these agents (e.g., while working) and suffer from injuries to the lung that lead to inflammation. Non-limiting examples of the environmental and industrial toxic agents include isocyanate (e.g., toluene diisocyanate), nitrogen oxide, sulfuric acid, ammonia, phosgene, diacetyl, 2,3-pentanedione, 2,3-hexanedione, fly ash, fiberglass, silica, coal dust, asbestos, hydrogen cyanide, cadmium, acrolein, acetaldehyde, formaldehyde, aluminum, beryllium, iron, cotton, tin oxide, bauxite, mercury, sulfur dioxide, zinc chloride, polymer fumes, and metal fumes (e.g., fumes generated by copper, magnesium, nickel, silver, or zinc). In some specific embodiments, the toxic-inhalation lung injury is pneumoconiosis. As used herein, pneumoconiosis refers to a class of interstitial lung diseases caused by inhalation of various solid particles. In some specific embodiments, the toxic-inhalation lung injury is bronchiolitis obliterans, commonly referred to as “popcorn lung.” In some specific embodiments, the bronchiolitis obliterans is caused by the inhalation of one or more industrial toxic agents selected from the group consisting of acetaldehyde, formaldehyde, diacetyl, 2,3-pentanedione, and 2,3-hexanedione.

In still other embodiments, the toxic-inhalation lung injury is a vaping-associated lung injury. Vaping, or using electronic cigarettes, may cause the user to inhale harmful chemicals and result in lung injuries. In recent years, there has been a significant growth in cases of vaping-associated lung injury reported in the U.S. In some embodiments, an electronic cigarette user inhales harmful chemicals found in the electronic cigarette liquid, e.g., diacetyl, which causes injuries, e.g., burn, inflammation, to the lung. Other non-limiting examples of harmful chemicals in the electronic cigarette causing vaping-associated lung injury include 2,3-pentanedione, nicotine, carbonyls, volatile organic compounds (e.g., benzene and toluene), trace metal elements, α-Tocopheryl acetate, and bacterial endotoxins and fungal glucans. In some embodiments, the vaping-associated lung injury is pneumonitis. In some specific embodiments, the vaping-associated lung injury is bronchiolitis obliterans, commonly referred to as “popcorn lung.”

In yet other embodiments, the inflammatory disorder is selected from the group consisting of bronchiolitis obliterans syndrome (BOS), chronic obstructive pulmonary disease (COPD), pulmonary langerhans cell histiocytosis, non-cystic fibrosis bronchiectasis, diffuse panbronchiolitis, acute respiratory distress syndrome (ARDS), reactive airways dysfunction syndrome (RADS), bronchiolitis obliterans organizing pneumonia (BOOP), restrictive allograft syndrome (RAS), pulmonary arterial hypertension (PAH), interstitial lung disease (ILD), idiopathic pulmonary fibrosis (IPF), and pneumonitis.

Pulmonary langerhans cell histiocytosis is a lung disease more commonly occurring in smokers. In some embodiments, the subject having pulmonary langerhans cell histiocytosis suffers from inflammation mediated by IL-1 and anakinra blocks the activities of IL-1α and IL-1β local to the lower airways and thereby effectively treat pulmonary langerhans cell histiocytosis.

In non-cystic fibrosis bronchiectasis and in diffuse panbronchiolitis disease, various biological pathways are activated which induce inflammation of the lung. In some embodiments, the subject having non-cystic fibrosis bronchiectasis of the lower airways suffers from inflammation mediated by IL-1 and anakinra blocks the activities of IL-1α, IL-1β local to the lower airways and thereby effectively treats non-cystic fibrosis bronchiectasis. In other embodiments, the subject having diffuse panbronchiolitis of the lower airways suffers from inflammation mediated by IL-1 and anakinra blocks the activities of IL-1α, IL-1β local to the lower airways and thereby effectively treats diffuse panbronchiolitis.

Acute respiratory distress syndrome (ARDS) is a life-threatening disease which can require immediate mechanical ventilation to prevent lung failure. In some embodiments, ARDS is associated with complications arising from viral infections caused by SARS-CoV-2, SARS-CoV, MERS-CoV, 229E, NL63, OC43, and HKU1. In some embodiments, the subject having ARDS suffers from inflammation mediated by IL-1 and anakinra blocks the activities of IL-1α, IL-1β local to the lower airways and thereby effectively treats ARDS.

In some embodiments, the human subject to be treated suffers from reactive airways dysfunction syndrome (RADS), which refers to a persistent asthma-like disorder precipitated by a single acute exposure to an inhaled irritant. In some embodiments, the subject having RADS suffers from inflammation mediated by IL-1 and anakinra blocks the activities of IL-1α, IL-1β local to the lower airways and thereby effectively treats RADS.

In some embodiments, the method comprises treating a human subject suffering from bronchiolitis obliterans organizing pneumonia (BOOP), which is a type of lung disease resulting from organizing pneumonia that invades the bronchioles (small airways through the lungs) and alveoli (tiny air sacs) of the lungs. BOOP causes inflammation of the bronchioles and alveoli of the lung. In some embodiments, the subject having BOOP suffers from inflammation mediated by IL-1 and anakinra blocks the activities of IL-1α, IL-1β local to the lower airways and thereby effectively treats BOOP.

In some embodiments, the method comprises treating a human subject suffering from restrictive allograft syndrome (RAS), which is a form of chronic lung allograft dysfunction (CLAD) after lung transplantation. The main characteristics of RAS include a persistent and unexplained decline in lung function and persistent parenchymal infiltrates. The median survival after diagnosis of RAS is 6 to 18 months, significantly shorter than other forms of CLAD. Treatment options are limited, as therapies used for BOS are typically ineffective at halting disease progression.

In some embodiments, the method comprises treating a human subject suffering from pulmonary arterial hypertension (PAH), which is a chronic and progressive disease leading to right heart failure and ultimately death if untreated. Right heart failure can lead to fluid retention, hepatic congestion, ascites, and peripheral edema. Remodeling of small pulmonary arteries via the proliferation of smooth muscle and endothelial cells may play a major role in the pathogenesis of PAH. This abnormal proliferation includes hypertrophy of the media and intima, and the formation of tumor-like lesions from endothelial cells in regions of pulmonary artery bifurcation (plexiform lesions).

### Interstitial Lung Disease (ILD)

In some embodiments, the method comprises treating a human subject suffering from interstitial lung disease (ILD) which is an umbrella term used to refer to hundreds of chronic lung disorders characterized by inflammation of the lung tissue. In some instances, causing progressive pulmonary scarring/fibrosis. Fibrosis progressively causes lung stiffness, contributing to a reduced ability of the air sacs to capture and carry oxygen into the bloodstream and eventually leads to permanent loss of the ability to breathe.

### Idiopathic Pulmonary Fibrosis (IPF)

In some embodiments, the method comprises treating a human subject suffering from idiopathic pulmonary fibrosis (IPF) which is an age-related chronic and progressive lung disease of unknown cause that has few treatment options. IPF is characterized by radiographically evident interstitial thickening predominantly affecting the lung bases and by progressive dyspnea and worsening of pulmonary function.

In some embodiments, the human subject to be treated suffers from BOS. In BOS, host T cells recognize foreign antigens and infiltrate the lung to induce acute rejection. BOS occurs following chronic rejection and infiltration of T cells, B cells, and innate immune cells. Immune cells promote fibrosis and airway occlusion. Injury activates DAMPs/PAMPs which induce inflammasome activation and IL-1 release. IL-1 drives the innate immune response which increases inflammatory cytokine production by macrophages, dendritic cells, and mast cells. IL-1 also increases neutrophil recruitment and effector function and stimulates release of IFN-γ from NK cells. IL-1 also stimulates adaptive immunity including promoting the generation of CD8+ T cells and release of cytotoxic granzyme B. The role of IL-1 in adaptive immunity also enhances CD4+ T cell populations and differentiation into Th17 helper T cells, aids in memory T cell functions and priming of T cells and promotes cytotoxic cytokine release.

In some embodiments, a human subject suffering from BOS is administered a rhIL-1Ra, such as anakinra, to the lower airways. In some embodiments, the formulation of ALTA-2530 includes anakinra as the IL-1Ra. In some embodiments, the rhIL-1Ra inhibits IL-1 signaling, blocking both IL-α and IL-1β signaling. FIG. 1 depicts a mechanism of action of an rhIL-1Ra, in a composition such as ALTA-2530, in which both innate and adaptive immune reactions are mediated by IL-1 signaling (both IL-α and IL-1β signaling).

In some embodiments, anakinra is administered via inhalation or via direct instillation into the lower airways. In certain embodiments, a delivery device is used to administer anakinra directly to the lower airways. Non-limiting examples of the delivery devices include a nebulizer, an inhaler, and a subminiature aerolizer. In some specific embodiments, the delivery device is a dry powder inhaler. In some specific embodiments, the delivery device is a vibrating mesh nebulizer.

In yet another aspect, a method of treating an inflammatory disorder of the respiratory tract is disclosed, including administering to a patient in need thereof the pharmaceutical composition according to any one of the embodiments described herein.

In some embodiments, the inflammatory disorder of the respiratory tract is an inflammatory disorder of the upper airways. In some embodiments, the inflammatory disorder is selected from the group consisting of a toxic-inhalation lung injury, pulmonary langerhans cell histiocytosis, non-cystic fibrosis bronchiectasis, diffuse panbronchiolitis, acute respiratory distress syndrome (ARDS), reactive airways dysfunction syndrome (RADS), bronchiolitis obliterans organizing pneumonia (BOOP), bronchiolitis obliterans syndrome (BOS), restrictive allograft syndrome (RAS), pulmonary arterial hypertension (PAH), idiopathic pulmonary fibrosis (IPF), interstitial lung disease (ILD), pneumonitis, primary graft dysfunction (PGD), and reperfusion injury.

In some embodiments, the toxic-inhalation lung injury is caused by inhalation of one or more chemical warfare agents. In some embodiments, the chemical warfare agent is selected from the group consisting of chlorine gas and sulfur mustard. In some embodiments, the toxic-inhalation lung injury is chlorine-induced bronchiolitis obliterans syndrome (BOS) and sulfur mustard-induced bronchiolitis obliterans syndrome (BOS).

In some embodiments, the toxic-inhalation lung injury is caused by inhalation of one or more environmental and/or industrial toxic agents. In some embodiments, the environmental and industrial toxic agents are selected from the group consisting of isocyanate, nitrogen oxide, morpholine, sulfuric acid, ammonia, phosgene, diacetyl, 2,3-pentanedione, 2,3-hexanedione, fly ash, fiberglass, silica, coal dust, asbestos, hydrogen cyanide, cadmium, acrolein, acetaldehyde, formaldehyde, aluminum, beryllium, iron, cotton, tin oxide, bauxite, mercury, sulfur dioxide, zinc chloride, polymer fumes, and metal fumes.

In some embodiments, the toxic-inhalation lung injury is pneumoconiosis or bronchiolitis obliterans. In some embodiments, the toxic-inhalation lung injury is a vaping-associated lung injury. In some embodiments, the vaping-associated lung injury is caused by inhalation of one or more agents selected from the group consisting of diacetyl, α-Tocopheryl acetate, 2,3-pentanedione, nicotine, carbonyls, benzene, toluene, metals, bacterial endotoxins, and fungal glucans.

In some embodiments, the inflammatory disorder is an inflammatory disorder of the lung. In some embodiments, the inflammatory disorder of the respiratory tract is an inflammatory disorder of the lower airways.

In some embodiments, a sustained exposure of the pharmaceutical composition in a lung epithelial lining fluid is between about 15 hours and about 100 hours. In some embodiments, the sustained exposure of the pharmaceutical composition in the lung epithelial lining fluid is at least 24 hours.

In some embodiments, the pharmaceutical composition is administered between about once per week and about three times per day. In some embodiments, the pharmaceutical composition is administered about once or twice daily.

In some embodiments, the pharmaceutical composition is administered via inhalation for between about 3 minutes and about 20 minutes.

In some embodiments, the pharmaceutical composition is administered at a dose of between about 0.1 mg/kg and about 2 mg/kg.

In some embodiments, the pharmaceutical composition binds with substantially similar affinity as an endogenous IL-1β or IL-1α ligand to an IL-1 type 1 receptor. In some embodiments, the pharmaceutical composition binds to an IL-1 type 1 receptor with affinity greater than about 100, greater than about 1000, or greater then above 10,000 compared to an endogenous IL-1β or IL-1α ligand.

### Second Therapeutic Agent

In some embodiments, the method described herein further includes administering a second therapeutic agent in combination with the protein or peptide interleukin-1 receptor antagonist (e.g., anakinra) to the human subject suffering from an inflammatory disorder of the lower airways. In some embodiments, the second therapeutic agent is selected from the group consisting of an anti-inflammatory agent, an antiviral agent, an antibacterial agent, an antibiotic, an antifungal compound, an amiloride, an antihistamine, an anticholinergic, a mucolytic, and a steroid. In some specific embodiments, the second therapeutic agent is rodatristat ethyl. In some specific embodiments, the second therapeutic agent is an inhaled, orally administered or parenterally administered drug that is considered as the standard of care or investigational for the indication treated.

In one embodiment, the protein or peptide interleukin-1 receptor antagonist (e.g., anakinra) may also be administered along with other active or pharmacologic agents, such as UTP, amiloride, antibiotics, antihistamines, anti-cholinergics, anti-inflammatory agents, and mucolytics (e.g., n-acetyl-cysteine). It may also be useful to administer the protein or peptide interleukin-1 receptor antagonist (e.g., anakinra) along with other therapeutic human proteins including but not limited to serine and other protease inhibitors, gamma-interferon, enkephalinase, nucleases, colony stimulating factors, albumin, and antibodies. The protein or peptide interleukin-1 receptor antagonist (e.g., anakinra) may be administered sequentially or concurrently with the one or more other pharmacologic agents. The amounts of the protein or peptide interleukin-1 receptor antagonist (e.g., anakinra) and pharmacologic agent depend, for example, on what type of drugs are used, the type of lower airways inflammatory disorder being treated, and the scheduling and routes of administration. Following administration of the protein or peptide interleukin-1 receptor antagonist (e.g., anakinra) to the mammal (e.g., a human), the mammal's physiological condition can be monitored in various ways well known to one of ordinary skill in the art.

In another embodiment, the composition used for treating disorders of the lower airways may comprise a protein or peptide interleukin-1 receptor antagonist (e.g., anakinra or ALTA-2530 which is a composition that in some embodiments includes anakinra) and other compounds including but not limited to a mucoregulatory compound, a corticosteroid, a surfactant, an anticholinergic compound, a bronchodilator, a nuclease, an antibiotic, an antiviral agent, and an antiangiogenic agent. Additional examples of the second therapeutic agents are disclosed in U.S. Pat. No. 8,940,683, cols. 23-32 and 47-51, the content of which is expressly incorporated herein by reference.

### Pharmaceutical Compositions

This disclosure also provides a pharmaceutical composition comprising a protein or peptide interleukin-1 receptor antagonist (e.g., anakinra) and a pharmaceutically acceptable carrier.

In some embodiments, anakinra is administered in a pharmaceutical composition comprising anakinra and a pharmaceutically acceptable carrier. In some embodiments, the pharmaceutical composition is a spray, aerosol, gel, solution, emulsion, or suspension.

The composition is preferably administered to the mammal in a pharmaceutically acceptable carrier. Typically, in some embodiments, an appropriate amount of a pharmaceutically acceptable salt is used in the formulation to render the formulation isotonic.

In some embodiments, the pharmaceutically acceptable carrier is selected from the group consisting of saline, Ringer's solution, dextrose solution, and a combination thereof. Other suitable pharmaceutically acceptable carriers known in the art are contemplated. Suitable carriers and their formulations are described in Remington's Pharmaceutical Sciences, 2005, Mack Publishing Co. The pH of the solution is preferably from about 5 to about 8, and more preferably from about 7 to about 7.5. The formulation may also comprise a lyophilized powder. Further carriers include sustained release preparations such as semipermeable matrices of solid hydrophobic polymers, which matrices are in the form of shaped articles, e.g., films, liposomes or microparticles. It will be apparent to those persons skilled in the art that certain carriers may be more preferable depending upon, for instance, the route of administration and concentration of anakinra being administered.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier is “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term “carrier” denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency. The composition may also include additional agents such as an isotonicity agent, a preservative, a surfactant, and, a divalent cation, preferably, zinc.

The composition can also include an excipient, or an agent for stabilization of at least one proinflammatory cytokine inhibitor (e.g., anakinra) composition, such as a buffer, a reducing agent, a bulk protein, amino acids (such as e.g., histidine, glycine or praline) or a carbohydrate. Bulk proteins useful in formulating at least one proinflammatory cytokine inhibitor composition proteins include albumin. Typical carbohydrates useful in formulating anakinra include but are not limited to sucrose, mannitol, lactose, trehalose, or glucose.

Surfactants may also be used to prevent soluble and insoluble aggregation and/or precipitation of proteins included in the composition. Suitable surfactants include but are not limited to sorbitan trioleate, soya lecithin, and oleic acid. In certain cases, solution aerosols are preferred using solvents such as ethanol. Thus, formulation including anakinra can also include a surfactant that can reduce or prevent surface-induced aggregation of anakinra caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001% and 4% by weight of the formulation. Especially preferred surfactants for purposes of this invention are polyoxyethylene sorbitan mono-oleate, polysorbate 80, polysorbate 20. Additional agents known in the art can also be included in the composition.

In some embodiments, the pharmaceutical compositions and dosage forms further comprise one or more compounds that reduce the rate by which an active ingredient will decay, or the composition will change in character. So called “stabilizers” or “preservatives” and may include, but are not limited to, amino acids, antioxidants, pH buffers, or salt buffers. Nonlimiting examples of antioxidants include butylated hydroxy anisole (BHA), ascorbic acid and derivatives thereof, tocopherol and derivatives thereof, butylated hydroxy anisole and cysteine. Nonlimiting examples of preservatives include parabens, such as methyl or propyl p-hydroxybenzoate and benzalkonium chloride. Additional nonlimiting examples of amino acids include glycine or proline.

The present invention also teaches the stabilization (preventing or minimizing thermally or mechanically induced soluble or insoluble aggregation and/or precipitation of an inhibitor protein) of liquid solutions containing a proinflammatory cytokine inhibitor (e.g., anakinra) at neutral pH or less than neutral pH by the use of amino acids including proline or glycine, with or without divalent cations resulting in clear or nearly clear solutions that are stable at room temperature or preferred for pharmaceutical administration.

In one embodiment, the composition is a pharmaceutical composition of single unit or multiple unit dosage forms. Pharmaceutical compositions of single unit or multiple unit dosage forms of the invention comprise a prophylactically or therapeutically effective amount of one or more compositions (e.g., a compound of the invention, or other prophylactic or therapeutic agent), typically, one or more vehicles, carriers, or excipients, stabilizing agents, and/or preservatives. Preferably, the vehicles, carriers, excipients, stabilizing agents and preservatives are pharmaceutically acceptable.

In some embodiments, the pharmaceutical compositions and dosage forms comprise anhydrous pharmaceutical compositions and dosage forms. Anhydrous pharmaceutical compositions and dosage forms of the invention can be prepared using anhydrous or low moisture containing ingredients and low moisture or low humidity conditions. An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Suitable vehicles are well known to those skilled in the art of pharmacy, and non-limiting examples of suitable vehicles include glucose, sucrose, starch, lactose, gelatin, rice, silica gel, glycerol, talc, sodium chloride, dried skim milk, propylene glycol, water, sodium stearate, ethanol, and similar substances well known in the art. Saline solutions and aqueous dextrose and glycerol solutions can also be employed as liquid vehicles. Whether a particular vehicle is suitable for incorporation into a pharmaceutical composition or dosage form depends on a variety of factors well known in the art including, but not limited to, the way in which the dosage form will be administered to a patient and the specific active ingredients in the dosage form. Pharmaceutical vehicles can be sterile liquids, such as water and oils, including those of petroleum, animal, vegetable or synthetic origin, such as peanut oil, soybean oil, mineral oil, sesame oil and the like.

A pharmaceutical composition of the invention is formulated to be compatible with its intended route of administration. Examples of routes of administration within the lower airways include, but are not limited to, oral or nasal inhalation (e.g., inhalation of sufficiently small particles to be deposited expressly within the lower airways). In various embodiments, the pharmaceutical compositions or single unit dosage forms are sterile and in suitable form for administration to a subject, preferably an animal subject, more preferably a mammalian subject, and most preferably a human subject.

The composition, shape, and type of dosage forms of the invention will typically vary depending on their use. Non limiting examples of dosage forms include powders; solutions; aerosols (e.g., sprays, metered or nonmetered dose atomizers, oral or nasal inhalers including metered dose inhalers (MDI)); liquid dosage forms suitable for mucosal administration to a patient, including suspensions (e.g., aqueous or non-aqueous liquid suspensions, oil-in-water emulsions, or a water-in-oil liquid emulsions), solutions, and solids (e.g., crystalline or amorphous solids) that can also be reconstituted to provide liquid dosage forms suitable for lower airways administration. Formulations in the form of powders or granulates may be prepared using the ingredients mentioned above in a conventional manner using, e.g., a mixer, a fluid bed apparatus or a spray drying equipment.

The invention also provides that a pharmaceutical composition can be packaged in a hermetically sealed container such as an ampoule (e.g., blow-fill-seal (BFS) container) or sachette. In one embodiment, the pharmaceutical composition can be supplied as a dry sterilized lyophilized powder in a delivery device suitable for administration to the lower airways of a patient. In one embodiment, the pharmaceutical composition can be spray dried and supplied as a dry powder. The pharmaceutical compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for administration may be in the form of powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and the like, each containing a predetermined amount of a compound of the present invention (e.g., anakinra) as an active ingredient.

A liquid composition herein can be used as such with a delivery device, or they can be used for the preparation of pharmaceutically acceptable formulations comprising anakinra that are prepared for example by the method of spray drying. The methods of spray freeze-drying proteins for pharmaceutical administration disclosed in Maa et al., Curr. Pharm. Biotechnol., 2001, 1, 283-302, are incorporated herein. In another embodiment, the liquid solutions herein are freeze spray dried and the spray-dried product is collected as a dispersible anakinra-containing powder that is therapeutically effective when administered into the lower airways of an individual.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another anticancer agents).

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### Dosage Forms

The current invention provides for dosage forms comprising a proinflammatory inhibitor (e.g., anakinra) suitable for treating inflammatory disorders within the upper and lower airways. The dosage forms can be formulated, e.g., as sprays, aerosols, nanoparticles, liposomes, or other forms known to one of skill in the art. See, e.g., Remington's Pharmaceutical Sciences; Remington: The Science and Practice of Pharmacy supra; Pharmaceutical Dosage Forms and Drug Delivery Systems by Howard C., Ansel et al., Lippincott Williams & Wilkins; 7th edition (Oct. 1, 1999).

Generally, a dosage form used in the acute treatment of a disorder may contain larger amounts of one or more of the active ingredients it comprises than a dosage form used in the chronic treatment of the same disease. In addition, the prophylactically and therapeutically effective dosage form may vary among different types of disorders. For example, a therapeutically effective dosage form may contain a compound that has an appropriate antibacterial action when intending to treat a lower airways disorder associated with a bacterial infection. These and other ways in which specific dosage forms encompassed by this invention will vary from one another and will be readily apparent to those skilled in the art. See, e.g., Remington's Pharmaceutical Sciences, 2005, Mack Publishing Co.; Remington: The Science and Practice of Pharmacy by Gennaro, Lippincott Williams & Wilkins; 20th edition (2003); Pharmaceutical Dosage Forms and Drug Delivery Systems by Howard C. Ansel et al., Lippincott Williams & Wilkins; 7th edition (Oct. 1, 1999); and Encyclopedia of Pharmaceutical Technology, edited by Swarbrick, J. & J. C. Boylan, Marcel Dekker, Inc., New York, 1988, which are incorporated herein by reference in their entirety.

Suitable excipients (e.g., carriers and diluents) and other materials that can be used to provide dosage forms encompassed by this invention are well known to those skilled in the pharmaceutical sciences and depend on the particular tissue to which a given pharmaceutical composition or dosage form will be applied. With that in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Emulsifying agents, preservatives, antioxidants, gel-forming agents, chelating agents, moisturizers, or humectants can also be added to pharmaceutical compositions and dosage forms if desired. Examples of such additional ingredients are well known in the art. See, e.g., Remington's Pharmaceutical Sciences; Remington: The Science and Practice of Pharmacy; Pharmaceutical Dosage Forms and Drug Delivery Systems, supra.

Powders and sprays can contain, in addition to a compound of this invention (e.g., anakinra), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and butane.

In a specific embodiment, the invention provides formulations for administration to the lower airways. Typically, the composition comprises an active compound(s) in combination with vehicles or the active compound is incorporated in a suitable carrier system. Pharmaceutically inert vehicles and/or excipients for the preparation of the composition include, e.g., buffering agents such as boric acid or borates, pH adjusting agents to obtain optimal stability or solubility of the active compound, lactose as a carrier, tonicity adjusting agents such as sodium chloride or borates, viscosity adjusting agents such as hydroxypropyl cellulose, methylcellulose, polyvinylpyrrolidone, polyvinyl alcohols or polyacrylamide, oily vehicle such as vehicles comprising arachis oil, castor oil and/or mineral oil. Emulsions and suspensions of the active drug substance may also be presented in the composition. In these cases, the composition may furthermore comprise stabilizing, dispersing, wetting, emulsifying and/or suspending agents.

Additional components may be used prior to, in conjunction with, or subsequent to treatment with active ingredients of the invention (e.g., anakinra). For example, penetration enhancers can be used to assist in delivering the active ingredients to the tissue. Suitable penetration enhancers include but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); and urea.

The pH of a pharmaceutical composition or dosage form may also be adjusted to improve delivery and/or stability of one or more active ingredients. Similarly, the polarity of a solvent carrier, its ionic strength, or tonicity can be adjusted to improve delivery. Compounds such as stearates can also be added to pharmaceutical compositions or dosage forms to alter advantageously the hydrophilicity or lipophilicity of one or more active ingredients to improve delivery. In this regard, stearates can also serve as a lipid vehicle for the formulation, as an emulsifying agent or surfactant. Different salts, hydrates, or solvates of the active ingredients can be used to adjust further the properties of the resulting composition.

### Administration to a Subject

The amount of the compound or composition of the invention that will be effective in conjunction with a particular method will vary, e.g., with the nature and severity of the disorder and the device by which the active ingredient(s) is administered. The frequency and dosage will also vary according to factors specific for each subject, such as age, body, weight, response, and the past medical history of the subject. Effective doses may be extrapolated from dose-response curves derived from in vitro or animal model test systems. Suitable regimens can be selected by one skilled in the art by considering such factors and by following, for example, dosages reported in the literature and recommended in the Physician's Desk Reference (60th ed., 2006).

In general, the recommended daily dose range of a compound of the invention for the conditions described herein lie within the range of from about 0.01 mg to about 200 mg per day, given as a single once-a-day dose preferably or as divided doses throughout a day. In one embodiment, the daily dose is administered twice or three times daily in equally divided doses. Specifically, a daily dose range should be from about 100 micrograms to about 150 milligrams per day, more specifically, between about 50 milligrams and about 80 milligrams per day. It may be necessary to use dosages of the active ingredient outside the ranges disclosed herein in some cases, as will be apparent to those of ordinary skill in the art. Furthermore, it is noted that in instances where a clinician or treating physician is involved, such a person will know how and when to interrupt, adjust, or terminate therapy in conjunction with individual subject response.

In some embodiments, the effective amount of the IL-1Ra (e.g., anakinra) is from about 0.1 mg to about 100 mg per day, from about 0.1 mg to about 150 mg per day, or from about 01 mg to about 80 mg per day. Effective dosages and schedules for administering the composition may be determined empirically, and making such determinations is within the skill in the art. Those skilled in the art will understand that the dosage of any composition that must be administered will vary depending on, for example, the mammal which will receive the composition, the route of administration, the particular composition used including the co-administration of other drugs and other drugs being administered to the mammal.

Alternatively, the dosage administered (e.g., of the IL-1Ra) can vary depending upon known factors, such as the pharmacodynamic characteristics of the particular agent, and its mode and route of administration; age, and health of the recipient; nature and extent of symptoms, kind of concurrent treatment, frequency of treatment, and the effect desired. As an example, treatment of mammals can be provided as a one-time or periodic dosage of the IL-1Ra (e.g., anakinra) from 0.01 to 200 mg, or 0.01 to 100 mg, such as 0.025, 0.05, 0.075, 0.1, 0.125, 0.25, 0.50, 0.75, 1.0, 1.125, 1.25, 1.5, 2, 2.5, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90, 100, 110, 120, 130, 140, 150, 160, 170, 180, 190, 200 mg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses. It will furthermore be appreciated that a therapeutically effective amount of a particular composition can be determined by those of ordinary skill in the art with due consideration of the factors pertinent to the subject. In some specific embodiments, the effective amount of the IL-1Ra (e.g., anakinra) is from about 5 mg to 80 mg per day.

Different therapeutically effective amounts of a specific composition may be applicable for different diseases, as will be readily known by those of skill in the art. Similarly, different therapeutically effective compounds may be included in a specific composition depending on the subject's disease. Similarly, amounts sufficient to prevent, manage, treat or ameliorate such disorders, but insufficient to cause, or sufficient to reduce, adverse effects associated with the compounds of the invention are also encompassed by the above-described dosage amounts and dose frequency schedules. Further, when a subject is administered multiple dosages of a compound or compositions of the invention, not all of the dosages need be the same. For example, the dosage administered to the subject may be increased to improve the prophylactic or therapeutic effect of the compound or it may be decreased to reduce one or more side effects that a particular subject is experiencing.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In certain embodiments, where a physician or clinical visit is involved, two or more therapies (e.g., prophylactic or therapeutic agents) are administered within the same subject visit. The therapies can be administered simultaneously.

In certain embodiments, one or more compounds of the invention and one or more other the therapies (e.g., prophylactic or therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound of the invention may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated, and the administration may be separated by at least at least 1 day, 2 days, 3 days, 5 days, 10 days, 15 days, 30 days, 45 days, 2 months, 75 days, 3 months, or 6 months.

In a specific embodiment, the invention provides a method of preventing or treating a disorder, (e.g., lower airways inflammatory disorders, or symptoms thereof) comprising administering to a subject in need thereof a dose of at least 100 micrograms, preferably at least 250 micrograms, at least 500 micrograms, at least 1000 micrograms, at least 10 milligrams, at least 20 milligrams, at least 30 milligrams, at least 40 milligrams, at least 50 milligrams, at least 60 milligrams, at least 70 milligrams, at least 80 milligrams, or more of one or more compounds of the invention once every 3 days, preferably, once every 4 days, once every 5 days, once every 6 days, once every 7 days, once every 8 days, once every 10 days, once every two weeks, once every three weeks, or once a month.

### Articles of Manufacture

The invention encompasses articles of manufacture. A typical article of manufacture of the invention comprises a unit dosage form of a composition or compound of the invention. In one embodiment, the unit dosage form is a container, preferably a sterile container, containing an effective amount of a composition or compound of the invention and a pharmaceutically acceptable carrier or excipient. The article of manufacture can further comprise a label or printed instructions regarding the use of composition or compound or other informational material that advises the physician, technician, consumer, subject, or patient on how to prevent, treat or derive beneficial result pertaining to the disorder in question. The article of manufacture can include instructions indicating or suggesting a dosing regimen including, but not limited to, actual doses, monitoring procedures, and other monitoring information. The article of manufacture can also further comprise a unit dosage form of another prophylactic or therapeutic agent, for example, a container containing an effective amount of another prophylactic or therapeutic agent. In a specific embodiment, the article of manufacture comprises a container containing an effective amount of a composition or compound of the invention and a pharmaceutically acceptable carrier or excipient and a container containing an effective amount of another prophylactic or therapeutic agent and a pharmaceutically acceptable carrier or excipient. Examples of other prophylactic or therapeutic agents include, but are not limited to, those listed above. Preferably, the packaging material and container included in the article of manufacture are designed to protect the stability of the product during storage and shipment.

Article of manufacture of the invention can further comprise devices that are useful for administering the unit dosage forms. Examples of such devices include, but are not limited to, syringes, dry powder inhalers, metered dose and nonmetered dose inhalers, and nebulizers.

Articles of manufacture of the invention can further comprise pharmaceutically acceptable vehicles or consumable vehicles that can be used to administer one or more active ingredients (e.g., a compound of the invention). For example, if an active ingredient is provided in a solid form that is to be reconstituted for lower airways administration, the article of manufacture can comprise a sealed container of a suitable vehicle in which the active ingredient can be dissolved. For lower airways administration, a particulate-free sterile solution is preferred. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

In another embodiment of the invention, articles of manufacture and kits are provided containing materials useful for treating the pathological conditions described herein and associated problems. The article of manufacture comprises a container with a label. Suitable containers include, for example, bottles, vials, and test tubes. The containers may be formed from a variety of materials such as glass or plastic. The container holds a composition having at least one active compound that is effective for treating, for example, inflammatory disorders. The active agent in the composition is a proinflammatory cytokine inhibitor, and the composition may contain one or more active agents. The label on the container indicates that the compositions is used for treating, for example, lower airways inflammatory disorders, and may indicate directions for in vivo use, such as those described above.

In some embodiments, articles of manufacture and kits are provided that specifically incorporate an inhaler. The inhaler preferably is effective at delivering a compound or composition of the invention to specific sites within the lower airways, while minimizing drug distribution to the pharynx and upper airways. The delivery device may incorporate certain parts including but not limited to filters, needles, syringes, valves, atomizers, nasal adapters, electronic nebulizers, meters, heating elements, reservoirs, a power source(s); and package inserts with instructions for use.

The kit of the invention comprises the container described above and may also include a second or third container comprising a pharmaceutically acceptable carrier or buffer, dosing reservoir, or a surfactant. It may further include other materials desirable from a commercial and user standpoint, including other buffers, diluents, and a device for delivery expressly to the lower airways incorporating filters, needles, syringes, valves, atomizers; and package inserts with instructions for use.

### Delivery Devices

A general aspect of the current invention is the local delivery expressly to the lower airways of the composition and the delivery device that accomplishes said dosing. Delivery devices of the current invention provide methods for the local delivery of the composition whereby one or more pharmacologically active agents or local treatments of the composition may have local effects expressly in the vicinity of the mucosa of the lower airways. The advantages of local therapy for local disease include the lack of adverse effects due to systemic exposure of the active ingredient.

For pulmonary administration, preferably at least one proinflammatory cytokine inhibitor (e.g., anakinra) is delivered in a particle size effective for reaching the lower airways. There are a several desirable features of an inhalation device for administering the proinflammatory cytokine inhibitors and compositions of the present invention. To be specific, delivery by the inhalation device is generally reliable, reproducible, and accurate. The inhalation device can optionally deliver small dry particles, e.g., less than about 10 microns, preferably about 3 to 5 microns, for good respirability, or dry particles with small stokes radius.

According to the invention, at least one pro-inflammatory cytokine inhibitor (e.g., anakinra) can be delivered by any of a variety of inhalation devices known in the art for administration of a therapeutic agent by inhalation. These devices capable of depositing aerosolized formulations in the lower airways of a patient include but are not limited to metered dose inhalers, sprayers, nebulizers, and dry powder generators. Other devices suitable for pulmonary administration of proteins and small molecules, including proinflammatory cytokine inhibitors, are also known in the art. All such devices can use of formulations suitable for the dispensing of proinflammatory cytokine inhibitors in an aerosol. Such aerosols can include nanoparticles, microparticles, solutions (both aqueous and nonaqueous), or solid particles.

Nebulizers like AERx Aradigm, the Ultravent nebulizer (Mallinckrodt), and the Acorn II nebulizer (Marquest Medical Products) (U.S. Pat. No. 5,404,871, PCT Publication No. WO 1997/22376, entirely incorporated herein by reference), produce aerosols from solutions. In some embodiments, the nebulizer is Monaghan Aeroeclipse II Breath Activated Jet Nebulizer, or the Philips InnoSpire GO—a vibrating mesh nebulizer. In some embodiments, the nebulizer is a next-generation Philips device that use a mesh, such as the iNeb AAD and the iNeb Advance. iNeb AAD is in labeled use in the U.S. for Ventavis (Actelion) and in the EU for Promixin (colistin for CF), both are exclusive within indication, and are in the label. In other embodiments, the nebulizer is the PARI eFlow® or the Aerogen Solo.

In some embodiments, any suitable dry powder inhalers that use breath-actuation of a mixed powder can be used, as known in the art (U.S. Pat. No. 4,668,218, EP 237507, PCT Publication No. WO 1997/25086, PCT Publication No. WO 1994/08552, U.S. Pat. No. 5,458,135, and PCT Publication No. WO 1994/06498, all of which are herein entirely incorporated by reference).

These specific examples of commercially available inhalation devices are intended to be a representative of specific devices suitable for the practice of this invention and are not intended as limiting the scope of the invention. In some embodiments, a composition comprising at least one proinflammatory cytokine inhibitor (e.g., anakinra) is delivered by a dry powder inhaler or a sprayer. In other embodiments, a composition comprising at least one proinflammatory cytokine inhibitor (e.g., anakinra) is an aerosolized formulation delivered by an aerosolized nebulizer.

Once more, the composition of the present invention can be administered as a topical spray or powder to the lower airways of a mammal by a delivery device (e.g., oral or nasal inhaler, aerosol generator, oral dry powder inhaler, through a fiberoptic scope, or via syringe during surgical intervention). These numerous drug delivery devices capable of drug distribution to the lower airways can use a liquid, semisolid, and solid composition. Investigators have found the site of deposition in the lower airways and the deposition area depend on several parameters related to the delivery device, such as mode of administration, particle size of the formulation and velocity of the delivered particles. They describe several in vitro and in vivo methods that may be used by one of ordinary skill in the art to study distribution and clearance of therapeutics delivered to the lower airways, all of which is incorporated in its entirety, herein. Thus, any of these devices may be selected for use in the current invention, given one or more advantages for a particular indication, technique, and subject. These delivery devices include but are not limited to devices producing aerosols nebulizers and other metered and nonmetered inhalers.

In general, current container-closure system designs for inhalation spray drug products include both premetered and device-metered presentations using mechanical or power assistance and/or energy from patient inspiration for production of the spray plume. Premetered presentations may contain previously measured doses or a dose fraction in some type of units (e.g., single, multiple blisters, or other cavities) that are subsequently inserted into the device during manufacture or by the patient before use. Typical device-metered units have a reservoir containing formulation sufficient for multiple doses that are delivered as metered sprays by the device itself when activated by the patient.

An embodiment of the current invention is the use of a delivery device that is able to distribute the composition expressly to the mucosa of the lower airways in a subject in need of such treatment. In some embodiments, the delivery device is able to distribute the composition expressly to the mucosa of the lower airways in a subject in need of such treatment, with a small amount of composition reaching the pharynx and upper airways. In some embodiments, the delivery device is able to distribute the composition expressly to the mucosa of the lower airways in a subject in need of such treatment, with a minimal amount distributed to the posterior pharynx and the upper airways. In some embodiments, the delivery device is able to distribute the composition expressly to the mucosa of the lower airways in a subject in need of such treatment, with a negligible amount distributed to the posterior pharynx and the upper airways.

The current invention also incorporates multidose metering or nonmetering inhalers that are especially suited for repeated administrations and can provide numerous doses (typically 60 to up to about 130 doses, or more) either with or without stabilizers and preservatives.

Administration of a composition comprised of a proinflammatory cytokine inhibitor (e.g., anakinra) as a spray can be produced by forcing a suspension or solution of at least one proinflammatory cytokine inhibitor through a nozzle under pressure. The nozzle size and configuration, the applied pressure, and the liquid feed rate can be chosen to achieve the desired output and particle size to optimize deposition expressly in the lower airways. An electrospray can be produced, for example, by an electric field in connection with a capillary or nozzle feed. Advantageously, particles of at least one proinflammatory cytokine inhibitor composition delivered by a sprayer have a particle size less than about 20 microns, preferably in the range below 10 microns, and most preferably, about 3 to 5 microns, but other particle sizes may be appropriate depending on the device, composition, and subject needs.

Commercially available nebulizers for liquid formulations, including jet nebulizers and ultrasonic nebulizers may also be useful for administration to the lower airways. Liquid formulations may be directly nebulized, and lyophilized powder nebulized after reconstitution. In addition, the liquid formulation of composition may be instilled through a bronchoscope, placed directly into the affected regions.

One of ordinary skill in the art will recognize that the methods of the current invention can be achieved by lower airways administration of at least one proinflammatory cytokine inhibitor (e.g., anakinra) compositions via devices not described herein. The current invention also incorporates unit-dose metering and nonmetering spray devices that are especially suited for single administration. These devices are typically used for acute short-term treatments (i.e., acute exacerbations) and single-dose delivery (i.e., long-acting compositions) and can accommodate a liquid, powder, or mixture of both formulations of the composition. However, in certain circumstances, these unit dose devices may be preferred over multidose devices when used repeatedly in a particular way.

Another embodiment of the invention provides for a single-dose syringe prefilled with the composition appropriate for treating the lower airways inflammatory disease of the subject. Said prefilled syringe may be sterile or nonsterile and used in dose administration during procedures to a subject in need of lower airways therapy. An example of an application where a syringe is preferred includes, but is not limited to, the distribution of composition through an endoscope. These examples are not intended to be limiting and one skilled in the art will appreciate that other options exist for delivery of the composition expressly to the lower airways and these are incorporated herein.

In one embodiment of the invention, a composition containing one or more therapeutic agents described herein is directly administered to the lower airways. Such administration may be carried out via use of an intrapulmonary aerolizer, which create an aerosol containing the composition and which may be directly installed into the lower airways. Exemplary aerolizers are disclosed in U.S. Pat. Nos. 5,579,578; 6,041,775; 6,029,657; 6,016,800, and 5,594,987, all of which are herein incorporated by reference in their entirety. Such aerolizers are small enough in size so they can be inserted directly into the lower airways, for example into an endotracheal tube or even into the trachea. In one embodiment, the aerolizer may be positioned near the carina, or first bifurcation, of the lung. In another embodiment, the aerolizer is positioned so as to target a specific area of the lung, for example an individual bronchus, bronchiole, or lobe. Since the spray of the device is directly introduced into the lungs, losses due to deposition of the aerosol due to deposition on the walls of the nasal passages, mouth, throat, and trachea are avoided. Optimally, the droplet size produced by such suitable aerolizer is somewhat larger than those produced by ultrasonic nebulizers. Therefore, the droplets are less likely to be exhaled and thus leading to a delivery efficiency of virtually 100%. In addition, the delivery of the compositions has a highly uniform pattern of distribution.

In one embodiment, such an intrapulmonary aerosolizer comprises an aerosolizer attached to a pressure generator for delivery of liquid as an aerosol and which can be positioned in close proximity to the lungs by being inserted into the trachea directly or into an endotracheal tube or bronchoscope positioned within the trachea. Such an aerolizer may operate at pressures of up to about 2000 psi and produces particles with a medium particle size of 12 μm.

In an alternate embodiment, such an intrapulmonary aerosolizer comprises a substantially elongated sleeve member, a substantially elongated insert, and a substantially elongated body member. The sleeve member includes a threaded inner surface, which is adapted to receive the insert, which is a correspondingly threaded member. The threaded insert provides a substantially helical channel. The body member includes a cavity on its first end, which terminates by an end wall at its second end. The end wall includes an orifice extending therethrough. The body member is connected with the sleeve member to provide the aerosolizer of the present invention. The aerosolizer is sized to accommodate insertion into the trachea of a subject for administration of compositions containing anakinra. For operation of the device, the aerosolizer is connected by a suitable tube with a liquid pressure driver apparatus. The liquid pressure driver apparatus is adapted to pass liquid material (e.g., a composition containing one or more proinflammatory cytokine inhibitor) therefrom which is sprayed from the aerosolizer. Due to the location of the device deep within the trachea, the liquid material is sprayed in close proximity to the lungs, with resulting improved penetration and distribution of the sprayed material in the lungs.

In an alternate embodiment, such an aerosolizer, sized for intratracheal insertion, is adapted for spraying a composition containing anakinra directly into the lower airways (e.g., in close proximity to the lungs). The aerosolizer is placed into connection with a liquid pressure driver apparatus for delivering of the liquid composition. The aerosolizer comprises a generally elongated sleeve member, which defines a first end and a second end and includes a longitudinally extending opening therethrough. The first end of the sleeve member is placed in connection with the liquid pressure driver apparatus. A generally elongated insert is also provided. The generally elongated insert defines a first end and a second end and is received within at least a portion of the longitudinally extending opening of the sleeve member. The insert includes an outer surface which has at least one substantially helical channel provided surrounding its outer surface which extends from the first end to the second end. The substantially helical channel of the insert is adapted to pass the liquid material, which is received by the sleeve member. A generally elongated body member is also included which is in connection with the sleeve member. The body member includes a cavity provided in its first end, which terminates at an end wall which is adjacent its second end. The end wall is provided having an orifice therein for spraying the liquid material, which is received from the insert. The portions of the sleeve member, insert and body member, in combination, are of sufficient size to allow for intratracheal insertion. A method of using such an aerosolizer includes the steps of connecting an aerosolizer with a first end of a hollow tube member and connecting the second end of the hollow tube member with the liquid pressure driver apparatus. The method further includes the steps of providing the aerosolizer in the trachea or into a member which is provided in the trachea, and then activating the liquid pressure driver apparatus for spraying a composition containing one or more proinflammatory cytokine inhibitors therefrom.

In an alternate embodiment, a powder dose composition containing one or more proinflammatory cytokine inhibitors is directly administered to the lower airways via use of a powder dispenser. Exemplary powder dispensers are disclosed in U.S. Pat. Nos. 5,513,630, 5,570,686 and 5,542,412, all of which are herein incorporated in their entirety. Such a powder dispenser is adapted to be brought into connection with an actuator, which introduces an amount of a gas for dispensing the powder dose. The dispenser includes a chamber for receiving the powder dose and a valve for permitting passage of the powder dose only when the actuator introduces the gas into the dispenser. The powder dose is passed from the dispenser via a tube to the lower airways of the subject. The powder dose may be delivered intratracheally, near the carina, which bypasses the potential for large losses of the powder dose to e.g., the mouth, throat, and trachea. In addition, in operation the gas passed from the actuator serves to slightly insufflate the lungs, which provides increased powder penetration. For the intratracheal insertion, the tube can be effected through an endotracheal tube in anesthetized, ventilated subjects, including animal or human patients, or in conscious subjects, the tube be inserted directly into the trachea preferably using a small dose of local anesthetic to the throat and/or a small amount of anesthetic on the tip of the tube, in order to minimize a “gag” response.

In one embodiment, a composition containing one or more therapeutic agents described herein is directly administered to the lower airways. Such administration may be carried out via use of an aerolizer, which create an aerosol containing the composition and which may be directly installed into the lower airways. Exemplary aerolizers are disclosed in U.S. Pat. Nos. 5,579,758; 6,041,775; 6,029,657; 6,016,800; 5,606,789; and 5,594,987 all of which are herein incorporated by reference in their entirety. The invention thus provides for the methods of administering compositions containing one or more proinflammatory cytokine inhibitors directly to the lower airways by an aerolizer.

In particular, an embodiment of the present invention is a new use for the “intratracheal aerosolizer” device which methodology involves the generation of a fine aerosol at the tip of a long, relatively thin tube that is suitable for insertion into the trachea. Thus, the present invention provides a new method of use for this aerosolizer technology in a microcatheter as adapted herein, for use in the lower airways in the prevention, treatment, and care of lower airways disorders.

In another embodiment of the invention, an aerosolizing microcatheter is used to administer a composition containing pro-inflammatory cytokine inhibitor. Examples of such catheters and their use, termed “intratracheal aerosolization,” which involves the generation of a fine aerosol at the tip of a long, relatively thin tube that is suitable for insertion into the trachea, are disclosed in U.S. Pat. Nos. 5,579,758; 5,594,987; 5,606,789; 6,016,800; and 6,041,775.

In a further embodiment, a new use for the microcatheter aerosolizer device (U.S. Pat. Nos. 6,016,800 and 6,029,657) is adapted for nasal and paranasal sinus delivery and uses to deliver bioactive agents (e.g., anakinra) in the treatment, prevention, and diagnosis of lower airways disorders. One advantage of this microcatheter aerosolizer is the potential small size (0.014″ in diameter), and thus capable of being easily inserted into the working channel of a human flexible (1 to 2 mm in diameter) or ridged endoscope and thereby directed partially or completely into the ostium of a paranasal sinus.

One of ordinary skill in the art will recognize that the methods of the current invention can be achieved by lower airways administration of anakinra composition via devices not described herein.

### Uses of the Compositions and Compounds of the Invention

The present invention provides methods for preventing, managing, treating, or ameliorating a disorder (e.g., inflammatory disorders of the lower airways) using a compound (e.g., anakinra) or composition of the invention in combination with another modality, such as a prophylactic or therapeutic agent known to be useful for, or having been or currently being used in the prevention, treatment, management, or amelioration of a disorder or used in the lessening of discomfort or pain associated with a disorder. Depending on the manner of use of the compositions or compounds, the invention can be co-administered with another modality, or the compositions or compounds of the invention can be mixed and then administered as a single composition to a subject. It is of course contemplated that the methods of the invention can be employed in combination with still other therapeutic uses such as surgical resection and lung transplantation.

In the methods, a composition is administered to a mammal diagnosed as having inflammatory disorder(s) of the lower airways. It is of course contemplated that the methods of the invention can be employed in combination with still other therapeutic techniques such as endoscopic monitoring and treatment techniques, surgical resection and lung transplantation.

Described herein are uses of the compounds and compositions of the invention for attaining a beneficial effect pertaining to lower airways inflammatory disorders, providing a beneficial effect pertaining to such disorders, or one or more symptoms thereof. The methods comprise administering to a subject in need thereof a prophylactically or therapeutically effective amount of, one or more compounds or a composition(s) of the invention. For example, administration of such compounds can be via one or more of the pharmaceutical compositions of the invention. It is of course contemplated that the methods of the invention can be employed in combination with oral or nasal inhalation devices. Importantly, it is contemplated that the methods of the invention can be employed in combination with the subcutaneous or intravenous injection, or other systemic routes of administration of proinflammatory cytokine inhibitors (e.g., anakinra). However, still other therapeutic techniques such as endoscopic procedures and treatment techniques, surgical resection and lung transplantation are all included herein.

In one embodiment, a subject in need of prevention, treatment, management, or amelioration of a disorder or a symptom thereof is a subject that has the disorder, that is known to be at risk of the disorder, has been diagnosed with the disorder, has previously recovered from the disorder, or is resistant to current therapy. In particular embodiments, the subject is an animal, preferably a mammal, and more preferably a human, that is predisposed and/or at risk because of a genetic factor(s), an environmental factor(s), or a combination thereof to develop the disorder. In yet another embodiment, the subject is refractory or non-responsive to one or more other treatments for a disorder. In yet another embodiment, the subject is an immunocompromised or immunosuppressed mammal, such as a human.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance which can be adapted to the practice of this invention in its various embodiments and equivalents thereof.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—ALTA-2530 Target Product Attributes (GLP/GMP)

In some embodiments, ALTA-2530 formulations have the product attributes as described herein. In some embodiments, ALTA-2530 is a solution for nebulization that will be delivered using a hand-held nebulizer. In some embodiments, ALTA-2530 is a solution for nebulization delivered using a Philips InnoSpire GO vibrating mesh (VM) nebulizer. In some embodiments, ALTA-2530 is a solution for nebulization that will be delivered using a preclinical nebulizer (e.g., Aerogen Solo VM nebulizer). In some embodiments, ALTA-2530 is an extemporaneously prepared solution formulation for nebulization that can be produced at the preclinical and clinical study sites and is stable to nebulization over the dosing period and a minimum in-use period of 24 hours. In some embodiments, ALTA-2530 has nebulization storage condition requiring refrigeration. In some embodiments, ALTA-2530 developed for GLP toxicology and GMP clinical studies will preferably be the same or comparable to avoid any bridging studies (e.g., excipients will not differ, and ratios will not exceed GLP qualification levels). In some embodiments, the impurity profiles of the nebulized GMP clinical formulation will be similar to and will not exceed the impurity limits qualified in the GLP preclinical studies. In some embodiments, the clinical formulation solution concentration(s) are suitable for delivering 10-40 mg from the VM nebulizer (expressed as drug charge to nebulizer) in less than 5 minutes and ideally within 2-3 minutes using the PART eFlow® or the Philips InnoSpire GO nebulizer. In some embodiments, ALTA-2530 has reproducible delivered and pulmonary lung dose to support the clinical programs as demonstrated by chemical and aerosol performance stability over the in-use period and anticipated dosing duration.

Table 1 shows a summary of key CMC activities and deliverables.

TABLE 1
CMC Activities and Deliverables

| CMC Activity | Key Deliverables |
|---|---|
| Analytical | Develop/optimize and qualify/validate phase |
| Method | appropriate analytical methods to support GLP; |
| Development | preclinical and GMP Phase 1 clinical programs for an |
| | ALTA-2530 solution for nebulization; |
| | Generate the associated analytical protocols and |
| | method development/validation reports; |
| | Generate final analytical methods; |
| | Provide supporting data for the extemporaneous |
| | product specification |
| Inhalation | Identify potential protein formulation matrices to be |
| Formulation | used in a preclinical tolerability study with the Kineret |
| Screening | matrix as a control using |
| | freeze/thaw studies; |
| | Identify preclinical formulations based on short-term |
| | stressed stability studies and the preclinical tolerability |
| | results; |
| | Conduct filter compatibility studies on lead and back- |
| | up formulations |
| Aerosol | Identify GLP (and GMP) solutions for nebulization |
| Characterization | that are stable to nebulization over the in-use and |
| | delivery periods using a preclinical nebulizer (e.g., |
| | Aerogen Solo) and a potential clinical nebulizer (e.g., |
| | modified PARI eFlow ® or Philips InnoSpire GO); |
| | Generate preparation instructions to be used by the |
| | preclinical site compounding pharmacy (e.g, a |
| | pharmacy manual); |
| | Specify the in-use period and storage conditions for |
| | the prepared solutions for nebulization; |
| | Compare impurity profiles from both nebulizers and |
| | provide findings in a QA reviewed development report |
| Pulmonary Dose | Estimate the pulmonary dose obtained from nebulized |
| Confirmation and | solutions of Kineret dilutions using the AeroEclipse II |
| In-Use Stability | breath-activated jet nebulizer, the PARI eFlow ® |
| Period - | nebulizer, and/or the Philips InnoSpire GO nebulizer; |
| Extemporaneous | Estimate the pulmonary dose and dose variability from |
| Preparations | nebulized solutions of ALTA-2530 using the modified |
| | PARI eFlow ® and/or Philips InnoSpire GO nebulizer |
| | over the in-use period and dosing duration |

### Example 2—ALTA-2530 Formulations

Table 2 shows different possible embodiments for ALTA-2530 formulations (e.g., comprising anakinra).

TABLE 2
Formulation Compositions

| Formulation | | | Concentration | | Role in anakinra |
|---|---|---|---|---|---|
| Components | Component | Options | Ranges | | formulations |
| Stabilizers | Surfactants | Polysorbate 80 | 0.1% | (0.01-1%) | Non-ionic |
| | | Polysorbate 20 | | | surfactant which |
| | | Brig 35 | | | reduces |
| | | | | | aggregation and |
| | | | | | denaturation, |
| | | | | | increases |
| | | | | | solubility |
| | Chelating | EDTA | 0.5 mM | (0.05-1 mM) | Stabilization |
| | agents | disodium | | | |
| | Sugars | Trehalose | >40% | w/v | Conformational |
| | | Sucrose | | | stability |
| | | Glycerol | | | |
| | | Sorbitol | | | |
| Buffers - | Buffers | Citrate | 20 | mM | Combined with |
| pH ~ 6.5-7.5 | | Phosphate | 10 mM | (1-50 mM) | mannitol |
| | | Histidine | 10 mM | (5-50 mM) | Combined with |
| | | Glutamate | 10 mM | (10-50 mM) | taurine and HP |
| | | Pyrophosphate | | | |
| | | HEPES | | | |
| Tonicity | Tonicity | Sodium chloride | 140 mM | (120-180 mM) | Combined with |
| modifiers | agents | Mannitol | 10 mg/mL | (5-50 mg/mL) | phosphate; No |
| | Osmolytes | Taurine | 30 mg/mL | (15-25 mg/mL) | Irritation |
| | | Hydroxyproline | 26 mg/mL | (15-26 mg/mL) | Combined with |
| | | Proline | | | HEPES |
| | | | | | Combined with |
| | | | | | HEPES |

Table 3 shows different possible excipients for various embodiments for ALTA-2530 formulations.

TABLE 3
Excipient Options

| | | Stabilizers | | | | Tonicity |
|---|---|---|---|---|---|---|
| | Buffers | Sugars | Chelators | Surfactants | Osmolytes | Agents |
| Formulation | Citrate | Trehalose | EDTA | Polysorbate 80 | Taurine | NaCl |
| Components | Phosphate | Sucrose | disodium | Polysorbate 20 | Hydroxyproline | |
| | Histidine | Mannitol | | Sorbitan | | |
| | HEPES | | | trioleate | | |
| | Glutamate | | | | | |

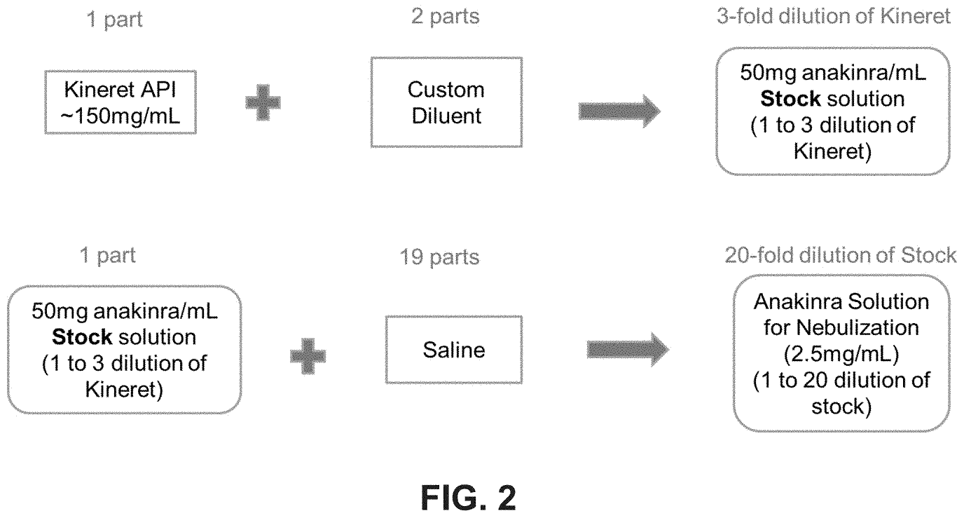

Table 4 shows a checkerboard for various embodiments of ALTA-2530 formulations. FIG. 2 shows the preparation procedure for ALTA-2530 solutions for nebulization studies.

TABLE 4
Checkerboard for various embodiments of ALTA-2530 formulations

| | | | | | Stabilizers | | | | | | | Osmolytes | |
|---|---|---|---|---|---|---|---|---|---|---|---|---|---|
| | | | | | | | | Chelators | | | | Taurine or | Tonicity |
| | | Buffers | | | Sugars | | | EDTA | Surfactants | | | hydroxy- | Agents |
| Matrix | Rationale | Citrate | Phosphate | Glutamate | Mannitol | Trehalose | Sucrose | Disodium | PS 80 | PS 20 | Span 85 | proline | NaCl |
| 1 | Citrate + | X | | | | | | X | X | | | | X |
| | PS80 | | | | | | | | | | | | |
| | (control) | | | | | | | | | | | | |
| 2 | Phos + | | X | | | | | X | X | | | | X |
| | PS80 | | | | | | | | | | | | |
| 3 | Phos + | | X | | X | | | X | | | | | X |
| | Mannitol | | | | | | | | | | | | |
| 4 | Phos + | | X | | X | | | X | X | | | | X |
| | Mannitol + | | | | | | | | | | | | |
| | PS80 | | | | | | | | | | | | |
| 5 | Phos + | | X | | X | | | X | | X | | | X |
| | Mannitol + | | | | | | | | | | | | |
| | PS20 | | | | | | | | | | | | |
| 6 | Phos + | | X | | X | | | X | | | X | | X |
| | Mannitol + | | | | | | | | | | | | |
| | Span 85 | | | | | | | | | | | | |
| 7 | Phos + | | X | | | X | | X | X | | | | X |
| | Trehalose + | | | | | | | | | | | | |
| | PS80 | | | | | | | | | | | | |
| 8 | Phos + | | X | | | | X | X | X | | | | X |
| | Sucrose + | | | | | | | | | | | | |
| | PS80 | | | | | | | | | | | | |
| 9 | Phos + | | X | | | | | X | | | | X | X |
| | Osmo | | | | | | | | | | | | |
| 10 | Phos + | | X | | X | | | X | X | | | X | X |
| | Osmo + | | | | | | | | | | | | |
| | Mannitol + | | | | | | | | | | | | |
| | PS80 | | | | | | | | | | | | |
| 11 | Phos + | | X | | | X | | X | | X | | X | X |
| | Osmo + | | | | | | | | | | | | |
| | Trehalose + | | | | | | | | | | | | |
| | PS20 | | | | | | | | | | | | |
| 12 | Phos + | | X | | | | X | X | | | X | X | X |
| | Osmo + | | | | | | | | | | | | |
| | Sucrose + | | | | | | | | | | | | |
| | Span85 | | | | | | | | | | | | |
| 13 | Citrate + | X | X | | | | | X | X | | | | X |
| | Phos + | | | | | | | | | | | | |
| | PS80 | | | | | | | | | | | | |
| 14 | Glutamate + | | | X | | | | X | X | | | | X |
| | PS80 | | | | | | | | | | | | |
| 15 | Glutamate + | | | X | X | | | X | X | | | | X |
| | Mannitol + | | | | | | | | | | | | |
| | PS80 | | | | | | | | | | | | |
| 16 | Phos + | | X | | X | | | | | | | | X |
| | Mannitol − | | | | | | | | | | | | |
| | EDTA | | | | | | | | | | | | |
| 17 | Phos + | | X | | | | | | | | | X | X |
| | Osmo − | | | | | | | | | | | | |
| | EDTA | | | | | | | | | | | | |

Table 5 shows additional embodiments of ALTA-2530 formulations (e.g., comprising anakinra).

TABLE 5
ALTA-2530 Formulation Matrices

| | Diluent | | | | Stabilizers | | | |
|---|---|---|---|---|---|---|---|---|
| | (WFI as base | | Buffers | | | | Chelators | |
| | for diluent | Active | (mM) | | Sugars | | (mM) | |
| | unless | (mM) | Citric | | (mM) | | EDTA | Surfactant |
| Matrix # | specified) | anakinra | Acid | Histidine | Trehalose | Sucrose | Disodium | PS 80 |
| 0 | Kineret | 8.69 | 10 | | | | 0.51 | 0.8 |
| 1 | Saline | | | | | | | |
| | (control) | | | | | | | |
| 2 | Histidine + | | | 7.52 | | | | |
| | Saline | | | | | | | |
| 3 | Histidine + | | | 7.52 | | | 0.53 | |
| | EDTA + | | | | | | | |
| | Saline | | | | | | | |
| 4 | Trehalose | | | | 300 | | | |
| 5 | Histidine + | | | 7.52 | 300 | | 0.53 | |
| | EDTA + | | | | | | | |
| | Trehalose | | | | | | | |
| 6 | Histidine + | | | 7.52 | | 285 | 0.53 | |
| | EDTA + | | | | | | | |
| | Sucrose | | | | | | | |
| 7 | Histidine + | | | 7.52 | 300 | | 0.53 | |
| | EDTA + | | | | | | | |
| | Trehalose + | | | | | | | |
| | Taurine | | | | | | | |
| 8 | Histidine + | | | 7.52 | 300 | | 0.53 | |
| | EDTA + | | | | | | | |
| | Trehalose + | | | | | | | |
| | HP | | | | | | | |

| | | | | Calculated | Formulation | |
|---|---|---|---|---|---|---|
| | Osmolytes | | Tonicity | Osmolality | Concentrations | |
| | (mM) | | Modifiers | (mOsm/kg) | (mg/mL) | |
| | | Hydroxy- | (mM) | Custom | 2.5 | 50 |
| Matrix # | Taurine | proline | NaCl | Diluent | (saline) | (stock) |
| 0 | | | 140 | 294 | | |
| 1 | | | 154 | 310 | 1a | 1S |
| 2 | | | 154 | 320 | 2a | 2S |
| 3 | | | 154 | 320 | 3a | 3S |
| 4 | | | | 300 | 4a | 4S |
| 5 | | | | 310 | 5a | 5S |
| 6 | | | | 340 | 6a | 6S |
| 7 | 36 | | | 340 | 7a | 7S |
| 8 | | 34 | | 340 | 8a | 8S |

### Example 3—Analytical Development

In some embodiments, key deliverables include develop/optimize and qualify/validate phase appropriate analytical methods to support GLP preclinical and GMP Phase 1 clinical programs for an ALTA-2530 solution for nebulization. All qualification/validation studies will be conducted in accordance with ICH guidelines.

In some embodiments, attributes and methods for product specification/stability include: Appearance, pH, osmolality; identification by peptide mapping; protein concentration by A280; purity by RPHPLC, SE-HPLC, reduced and non-reduced CE-SDS, and IEX-HPLC; foreign and particulate matter and subvisible particles; aerosol particle size distribution by NGI for a nebulized solution as listed in USP 601 using an appropriate duration of testing; delivered dose using breath simulator as listed in USP 1601 and USP 601 over the entire duration of dosing; Bioburden and endotoxin; Cell-based Bioassay for potency.

In some embodiments, information only test methods to support formulation development include: Circular dichroism, viscosity, surface tension, formulation density, droplet size and distribution by Malvern Spraytec or equivalent, dynamic light scattering (DLS), DSC, and turbidity. In some embodiments, support specifications for active and placebo extemporaneous products are obtained. In some embodiments, validation summary reports for all validated method activities are produced.

### Example 4—Inhalation Formulation Screening

To screen the inhalation formulations, the acceptable targeted solution formulation pH for the pulmonary route of administration is between pH 5-8. The solution osmolality is within physiological ranges (˜300 mOsm/kg). Excipients used are “acceptable” or “well characterized” by the pulmonary route and within the concentration ranges/doses listed within the FDA Inactive Ingredient List for approved pulmonary products. Preference is given to either parenteral grade excipients (if available) and/or inhalation grade excipients currently used in marketed products for inhalation in major markets, including the US, EU, and Japan. A tiered approach is used to evaluate the preformulations including a physical stability screening study, stress stability screening study, and a formulation filtration study. For preformulation screening, studies will be conducted to identify formulation matrices and stable ALTA-2530 solutions for nebulization to be used in aerosol characterization studies. A control formulation (Kineret) will be used as a reference throughout these studies.

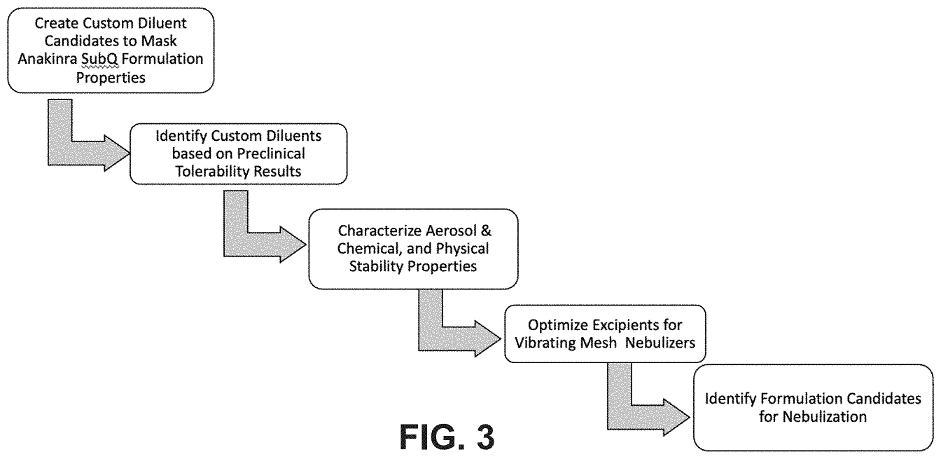

Preformulation studies are conducted in accordance with a tiered approach as shown in FIG. 3. In conducting the preformulation studies, screening studies are conducted to identify formulation matrices and stable ALTA-2530 (e.g., comprising anakinra) solutions for nebulization to be used in aerosol characterization studies. In some embodiments, ALTA-2530 (previously OSP-101) is a novel, inhaled composition of IL-1Ra to be delivered via an approved nebulizer. A control formulation (Kineret) is used as a reference. Kineret is approved for rheumatoid arthritis (USA, EU, Canada, Australia, etc.) and cryopyrin-associated periodic syndromes (up to 100 mg/day, subcutaneous injection). Formulation components include, but are not be limited to buffers, stabilizers, and tonicity modifiers (see Table 4). The buffers include histidine, phosphate, succinate, glutamate, citrate, PBS, and pyrophosphate. The stabilizers include polysorbate 20 and 80 and other compatible nonionic surfactants, EDTA disodium, glycerin, mannitol, and trehalose. The tonicity modifiers include sodium chloride and dextrose.

In conducting physical stability screening studies, approximately 10 formulations (various matrices+ALTA-2530 plus a Kineret control) using stressed conditions (e.g., freeze/thaw, agitation) are screened to identify potential protein formulation matrices to be used in a preclinical tolerability study. Characterization and output include physical and chemical characterization analyses of approximately 10 formulations (with ALTA-2530) (i.e., appearance, related substances, SEC, DSC, turbidity, DLS) after 1 to 2 freeze/thaw exposure(s) and agitation cycles.

In conducting stress stability screening studies, a solution formulation for use in GLP studies is evaluated using short term temperature/time stress-based stability.

In conducting formulation filtration studies, a lead and a back-up formulation is identified (up to 4 compositions; 2 matrices×2 concentrations) in the stressed testing screening studies. Filter compatibility studies (i.e., impurities and loss of content) using a maximum of 2×0.2 μm filter types is conducted. Results are generated using single and double filtration.

For physical stability screening studies, key deliverables include screening approximately 10 formulations (various matrices+ALTA-2530, Kineret control) using stressed conditions (e.g., freeze/thaw, agitation) to identify potential protein formulation matrices to be used in a preclinical tolerability study with the Kineret matrix as a control.

In some embodiments, characterization and output includes physical and chemical characterization analyses of approximately 10 formulations (with ALTA-2530) (i.e., appearance, related substances, SEC, DSC, turbidity, DLS) after 1 to 2 freeze/thaw exposure(s) and agitation cycles. In some embodiments, data is used to identify 4-6 matrices (without ALTA-2530) that are used in a preclinical tolerability study and/or used in short term stability studies. Preparation instructions and formulation components for the identified matrix compositions (without an IL-1Ra) are provided to the preclinical study site.

For stress stability screening studies, key deliverables are combined with outcomes from the preclinical tolerability study, as well as identifying a solution formulation for use in GLP studies by evaluating short term temperature/time stress-based stability. A dilution of this formulation is expected to be used in Phase 1 clinical studies. In some embodiments, 4-6 formulations (e.g., 2 concentrations of 2-3 formulations) by 2-3 storage conditions and freeze/thaw (if not conducted previously) are included; as well as initial time and 3 pull points (e.g., T=0, 24 hrs, 48 hrs, 7 days). In some embodiments, stress testing conditions are determined based on existing data (literature and physical stability screening study results).

For formulation filtration studies, key deliverables include using the lead and back-up formulations (up to 4 compositions; 2 matrices×2 concentrations) identified in the stressed testing screening studies, conduct filter compatibility studies (i.e., impurities and loss of content) using a maximum of 2×0.2 μm filter types. Results will be generated using single and double filtration.

### Example 5—Aerosol Characterization

Using formulations identified in preformulation screening studies from Example 4, stability to nebulization over the in-use period (T=0 and T=24 hr) and dosing duration to simulate clinical dosing using the Philips InnoSpire GO nebulizer is determined. A single nebulizer charge volume for these studies is also determined. Samples are evaluated from preclinical study site engineering runs to assess stability to nebulization over the anticipated dosing duration (i.e., duration of dosing for preclinical studies (e.g., 0, 1, 3 hrs)) using a preclinical nebulizer (Aerogen Solo).

To conduct the stability to nebulization study, a minimum of 2 and maximum of 4 solutions for nebulization (as identified during formulation screening studies) using both the clinical and preclinical nebulizers (if different) are characterized. An impurity profile as generated from the preclinical nebulizer over the intended dosing duration and in-use period (samples will be provided from engineering runs conducted at the preclinical study site) is determined. The impurity profile generated from a clinical nebulizer (e.g., modified PARI eFlow® or Philips InnoSpire GO) over the intended dosing duration and in use period is also determined. Pre-nebulization assessment of solution viscosity, density, turbidity, and surface tension data is collected and analyzed. Data is also collected for both nebulizers for each formulation at T=0 and T=24 hrs (solutions stored at refrigerated conditions) to determine: assay and impurities (pre- and post-nebulization by SEC and RP-HPLC); physical characterization (appearance and turbidity pre- and post-nebulization as collected nebulized solutions and solution remaining in nebulizer); VMD and GSD by Spraytec™; liquid output rate (LOR); and report time to empty, sputter, or clog nebulizer as well as the approximate residual volume in the nebulizer at this timepoint.

The pulmonary dose and dose variability using a minimum of 3 modified PART eFlow® or Philips InnoSpire GO nebulizer units over the in-use period for a maximum of 2 formulations (low and high solution concentrations, same matrix) and at 2 nebulizer charge volumes is estimated by generating APSD and GSD from 3 nebulizers; generating DD data (n=10) at a fixed duration (pre-determined time to sputter) using USP 1601; estimating the lung dose (using DD and cut-off of 5 μm and 3.5 μm APSD) and the dose variability; and estimating the lung dose as a function of multiple nebulizer charges for each fixed nebulizer charge volume.

In additional characterization studies, 10 formulations (plus 1 control formulation) are tested using stressed conditions (freeze/thaw and nebulization) to identify two to three custom diluents to be used in preclinical tolerability studies (see Table 6). The stressed conditions include two freeze/thaw cycles and nebulization using clinical nebulizer and associated formulation controls held at RT and protected from light. Each freeze-thaw cycle is one day. A five-day pull is added for storage at RT, and the formulation is protected from light to the stability plan. The characterization and output include physical and chemical characterization analyses of 11 formulations before and after each freeze/thaw exposure cycle and nebulization for: appearance, pH, A280, RP-HPLC, and SEC. Additional pre-nebulization analyses include viscosity, surface tension, and osmolality. Data is used to identify two to three placebo matrices (without an IL-1Ra) for use in a preclinical tolerability study and/or used in short term stability studies. Preparations of the identified matrix compositions (without an IL-1Ra) are provided to the preclinical study site.

TABLE 6
Stress stability study protocol

| Stress | | | Freeze/Thaw | Freeze/Thaw |
|---|---|---|---|---|
| Condition | T = 0 | Post Neb | Cycle 1 | Cycle 2 |
| Freeze/Thaw | X | n/a | (Y) | Y |
| Nebulization | | Y | n/a | n/a |

X = appearance, pH, A280, SEC, RP-HPLC, viscosity, surface tension, osmolality, DSD, LOR
Y = appearance, pH, A280, SEC, RP-HPLC

### Example 6—Viscosity and Protein Concentration on Aerosol Performance of ALTA-2530 Formulations

Rheology was used to demonstrate differences in viscosity across different control and ALTA-2530 formulations. Formulations 1-8 were tested for viscosity (see Table 5 for formulation information). FIG. 4 shows viscosity results for 8 formulations across 3 different concentrations of anakinra (2.5 mg/mL, 20 mg/mL, and 50 mg/mL; note that the 20 mg/mL concentration was only tested for formulation 5). FIG. 4 shows that viscosity increases with increasing anakinra concentration. Viscosity also increases in the presence of sugars in the 50 mg/mL concentration formulations. EDTA and histidine did not appear to impact viscosity.

Table 7 shows results for surface tension, viscosity, X50 (estimate for MMAD), Span, LOR, and FPF for the same 8 formulations across the 3 different anakinra concentrations (note that the 20 mg/mL concentration was only tested for formulation 5). Table 7 shows that higher viscosity results in smaller droplet sizes of the formulation which is important for targeting the deep airways of a subject.

TABLE 7
Characterization of various control and ALTA-2530 formulations

| | Custom | | | | | | | | FPF | FPF |
|---|---|---|---|---|---|---|---|---|---|---|
| | Diluent | | Conc | Surface | Viscosity | X50 | | | 5 μm | 3.3 μm |
| Matrix # | Description | Diluent | (mg/mL) | Tension | (cP) | (μm) | Span | LOR | (%) | (%) |
| 1 | Control | Saline | 2.5 | 44.44 | 0.99 | 4.50 | 1.79 | 0.61 | | |
| 1 | Control | Saline | 50 | 42.92 | 1.26 | 4.05 | 1.70 | 0.38 | | |
| 2 | Histidine | Saline | 2.5 | 45.46 | 0.96 | 4.40 | 1.83 | 0.60 | | |
| 2 | Histidine | Custom | 50 | 37.63 | 1.16 | 3.98 | 1.60 | 0.38 | 66.75 | 38.57 |
| 3 | Histidine + | Saline | 2.5 | 43.91 | 1.03 | 4.59 | 1.76 | 0.60 | | |
| | EDTA | | | | | | | | | |
| 3 | Histidine + | Custom | 50 | 36.25 | 1.15 | 4.05 | 1.74 | 0.46 | | |
| | EDTA | | | | | | | | | |
| 4 | Trehalose | Saline | 2.5 | 44.94 | 1.00 | 4.46 | 1.85 | 0.62 | | |
| 4 | Trehalose | Custom | 50 | 36.49 | 1.31 | 3.73 | 1.70 | 0.28 | 70.61 | 42.39 |
| 5 | Histidine + | Saline | 2.5 | 46.19 | 0.97 | 4.58 | 1.81 | 0.55 | | |
| | EDTA + | | | | | | | | | |
| | Trehalose | | | | | | | | | |
| 5 | Histidine + | Custom | 20 | ND | 1.28 | 4.41 | 1.72 | 0.49 | 58.43 | 33.86 |
| | EDTA + | | | | | | | | | |
| | Trehalose | | | | | | | | | |
| 5 | Histidine + | Custom | 50 | 41.63 | 1.44 | 3.87 | 1.71 | 0.28 | | |
| | EDTA + | | | | | | | | | |
| | Trehalose | | | | | | | | | |
| 6 | Histidine + | Saline | 2.5 | 43.49 | 0.96 | 4.47 | 1.84 | 0.60 | | |
| | EDTA + | | | | | | | | | |
| | Sucrose | | | | | | | | | |
| 6 | Histidine + | Custom | 50 | 38.78 | 1.42 | 3.75 | 1.69 | 0.28 | 70.32 | 42.10 |
| | EDTA + | | | | | | | | | |
| | Sucrose | | | | | | | | | |
| 7 | Histidine + | Saline | 2.5 | 46.71 | 0.96 | 4.56 | 1.79 | 0.60 | | |
| | EDTA + | | | | | | | | | |
| | Trehalose + | | | | | | | | | |
| | Taurine | | | | | | | | | |
| 7 | Histidine + | Custom | 50 | 42.34 | 1.34 | 3.72 | 1.76 | 0.27 | 70.29 | 42.79 |
| | EDTA + | | | | | | | | | |
| | Trehalose + | | | | | | | | | |
| | Taurine | | | | | | | | | |
| 8 | Histidine + | Saline | 2.5 | 44.01 | 0.92 | 4.73 | 1.73 | 0.60 | | |
| | EDTA + | | | | | | | | | |
| | Trehalose + | | | | | | | | | |
| | Hydroxyproline | | | | | | | | | |
| 8 | Histidine + | Custom | 50 | 46.33 | 1.46 | 3.78 | 1.69 | 0.25 | 69.82 | 41.76 |
| | EDTA + | | | | | | | | | |
| | Trehalose + | | | | | | | | | |
| | Hydroxyproline | | | | | | | | | |

| | | |
|---|---|---|
| Reference: | Water - average of start and | 71.99 |
| | end samples (HPLC grade) | |

Table 8 summarizes results for additional ALTA-2530 formulations. Table 8 shows that formulations 3 and 4 were the better performing formulations, both of which included trehalose.

TABLE 8
Characterization of various ALTA-2530 formulations

| Formulation | | |
|---|---|---|
| Number | Formulation | Observations |
| 1 | 10 mM Citrate, | Lower PD, turbidity, |
| | 140 mM NaCl, | and visible particles |
| | 0.53 mM EDTA, | than most formulations |
| | pH 6.5 | |
| 2 | 10 mM Citrate, | Highest PD, Turbidity, |
| | 0.53 mM EDTA, | and visible particles |
| | pH 6.5 | |
| 3 | 10 mM Citrate, | Second lowest PD and |
| | 300 mM Trehalose, | Turbidity, and no |
| | 0.53 mM EDTA, | visible particles |
| | pH 6.5 | |
| 4 | 10 mM Citrate, | Lowest PD and |
| | 75 mM NaCl, | Turbidity, and no |
| | 150 mM Trehalose, | visible particles |
| | 0.53 mM EDTA, | |
| | pH 6.5 | |
| 5 | 10 mM Citrate, | Second highest PD and |
| | 140 mM NaCl, | Turbidity, and visible |
| | 0.53 mM EDTA, | particles |
| | pH 5.0 | |
| 6 | 10 mM Citrate, | Small increase in DLS, |
| | 140 mM NaCl, | Appearance and |
| | 0.53 mM EDTA, | Turbidity results |
| | pH 5.5 | compared to control |
| 7 | 10 mM Phosphate, | Small differences across |
| | 140 mM NaCl, | formulations 7-9 seen in |
| | 0.53 mM EDTA, | DLS, Appearance and |
| | pH 6.0 | Turbidity results using a |
| 8 | 10 mM Phosphate, | draft surrogate |
| | 140 mM NaCl, | method |
| | 0.53 mM EDTA, | |
| | pH 6.5 | |
| 9 | 10 mM Phosphate, | |
| | 140 mM NaCl, | |
| | 0.53 mM EDTA, | |
| | pH 7.0 | |

FIG. 5 shows X50 (estimate for MMAD) for 8 formulations (see Table 5) across the three different concentrations of anakinra (2.5 mg/mL, 20 mg/mL, and 50 mg/mL; note that the 20 mg/mL concentration was only tested for formulation 5) using the Philips InnoSpire GO nebulizer. FIG. 5 demonstrates that increasing anakinra concentration reduces the MMAD.

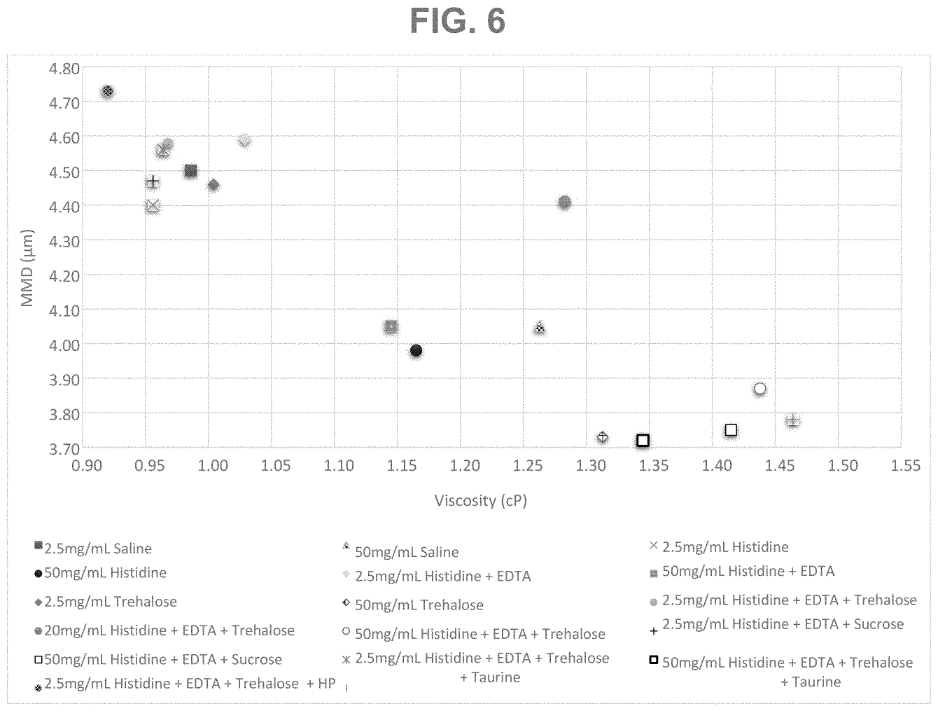

FIG. 6 shows X50 (estimate of MMAD) by viscosity for 8 different formulations (see Table 5) across the three different concentrations of anakinra (2.5 mg/mL, 20 mg/mL, and 50 mg/mL; note that the 20 mg/mL concentration was only tested for the histidine+EDTA+trehalose formulation) using the Philips InnoSpire GO nebulizer in order to examine the effect of sugar versus protein on the aerosol performance. FIG. 6 demonstrates that X50 decreases with increasing viscosity of the formulation. Viscosity increases with increasing protein concentration and sugar also impacts viscosity such that formulations including sugar increase viscosity and thereby reduce X50 (resulting in smaller droplet size).

FIG. 7 shows X50 (estimate of MMAD) for the 8 formulations shown in Table 5 at the 50 mg/mL protein concentration. The nebulization was performed using the Philips InnoSpire GO nebulizer. FIG. 7 shows that ALTA-2530 formulations (formulations 4-8) showed reduced X50 (smaller particle size) compared to the saline-based control formulations (formulations 1-3).

Therefore, it was surprisingly found that trehalose increases viscosity (and reduces droplet size) of ALTA-2530 formulations. In some embodiments, trehalose may stabilize the IL-1ra protein. Without being bound to a particular theory, it may be that because trehalose is a non-reducing sugar, it eliminates interaction with primary amines and stabilizes proteins due to its ability to hydrogen bond. In some embodiments, other non-reducing sugars may be used in ALTA-2530 formulations instead of trehalose. In some embodiments, sucrose can be used as the non-reducing sugar in the ALTA-2530 formulation. In some embodiments, if ALTA-2530 is prepared as a dry powder, trehalose may be preferred to sucrose as it may render more stability than sucrose due to a lower degree of molecular mobility.

In some embodiment, ALTA-2530 formulations include a non-reducing sugar at about 50 mM to about 100 mM, about 100 mM to about 150 mM, about 150 mM to about 200 mM, about 200 mM to about 250 mM, about 250 mM to about 300 mM, about 300 mM to about 350 mM, about 350 mM to about 400 mM, about 3400 mM to about 450 mM, about 450 mM to about 500 mM, about 500 mM to about 550 mM, or more than about 550 mM. In some embodiments, ALTA-2530 formulations include a non-reducing sugar at about 115 mM. In some embodiments, the non-reducing sugar is trehalose.

### Example 7—ALTA-2530 Formulation Characterization Across Vibrating Mesh Nebulizers

Modified PARI eFlow® Vibrating Mesh (VM) Nebulizer

An aerosol performance study was conducted by PARI in Germany using a modified PARI eFlow® Vibrating Mesh (VM) Nebulizer and a standard PARI eFlow® controller to nebulize 20 mg/mL and 50 mg/mL ALTA-2530 inhalation solutions. Confirmatory studies were performed to assess the aerosol performance as well as the stability to nebulization of 5 mg/mL and 20 mg/mL ALTA-2530 inhalation solutions using the same nebulizer configuration and controller and the analytical methods developed for the product. The formulations prepared included 5 mg/mL and 20 mg/mL of anakinra in ALTA-2530 inhalation solutions and a vehicle control (placebo diluted in custom diluent equivalent to 20 mg/mL active). The following materials were used in formulation preparation: ALTA-2530 drug substance (150 mg/mL), sodium chloride, USP/FCC/EP/BP histidine, USP disodium EDTA, USP trehalose, inhalation grade citric acid monohydrate, USP/FCC polysorbate 80 (HX2), USP/NF/EP/JP PARI eFlow® Vibrating Mesh (VM) Nebulizer, PARI eFlow® Controller, 0.2 μm PES filter assembly, scintillation vials, borosilicate glass, and 20 mL with urea screw caps. A custom diluent and placebo were prepared to formulate 5 mg/mL anakinra (ALTA-2530 composition), 20 mg/mL anakinra (ALTA-2530 composition), and a vehicle control. The custom diluent was composed of histidine, disodium EDTA, and trehalose. The protein supply was biosimilar to Kineret but containing larger PS80 concentrations. The placebo was composed of disodium EDTA, polysorbate 80, citric acid (anhydrous), and sodium chloride. The placebo matches the composition of the bulk drug substance (150 mg/mL ALTA-2530) without the IL1-Ra protein. The vehicle control was prepared by diluting the placebo in custom diluent to match the 20 mg/mL active formulation. All formulations were filtered using a 0.2 μm PES filter assembly into sterile bottles prior to filling into 20 mL borosilicate glass scintillation vials with urea screw caps closures under aseptic filling techniques.

The aerosol performance of ALTA-2530 used in the modified PARI eFlow® VM is shown in Table 9 below.

TABLE 9
Aerosol performance of ALTA-2530 in a modified PARI eFlow ® VM

| | Vehicle + | | | | | | |
|---|---|---|---|---|---|---|---|
| Aerosol | Saline | 20 mg/mL | | | | 5 mg/mL | |
| Performance | 4 mL | 1 mL | | 4 mL | | 4 mL | |
| DD per USP 1601, n = 10 | | n = 10; Nebs 2, | | n = 10 | | n = 10, Nebs 4 & 5 | |
| | | 3, 4 & 5 | | | | | |
| Mean DD (% of nominal) | | 80.1% | | Not | | 77.6% | |
| Std Dev (% of nominal) | | 3.58% | | Determined | | 3.15% | |
| DD Range (% nominal) | | 73.8%-85.3% | | | | 71.6%-81.6% | |
| Nebulization time (min), | | 5.80 min | (4.15-7.42 min) | | | 12.57 min | (9.53-17.38 min) |
| average time (range) | | | | | | | |
| Mass balance % (% RSD) | | 87.2% | (4.4%) | | | 90.1% | (1.8%) |
| LOR from DD for actives, | | n = 10; Nebs 2, | | | | n = 10, Nebs 4 & 5 | |
| n = 10 | | 3, 4 & 5 | | | | | |
| Mean (mL/min) | | 0.17 | mL/min | | | 0.31 | mL/min |
| Std Dev (% RSD) | | 0.033 | (19.6%) | | | 0.055 | mL/min (17.6%) |
| Range (mL/min) | | 0.13-0.23 | mL/min | | | 0.22-0.39 | mL/min |
| APSD by NGI, n = 5 | | n = 5; Nebs 2, | | n = 5; Nebs 2, | | n = 5, Nebs 2, | |
| | | 3 & 4 | | 3, 4 & 5 | | 4 & 5 | |
| mean MMAD (μm) | | 3.72 | μm | 3.89 | μm | 3.80 | μm |
| Std Dev (% RSD) | | 0.30 | μm (8%) | 0.34 | μm (9%) | 0.24 | μm (6%) |
| MMAD range (μm) | | 3.38 μm-4.04 μm | | 3.34 μm-4.18 μm | | 3.39 μm-4.04 μm | |
| GSD (Stdev) % RSD | | 1.57 | (0.04) 3% | 1.66 | (0.04) 3% | 1.58 | (0.03) 2% |
| Mass balance % (% RSD) | | 98.5% | (1%) | 88.7% | (2%) | 96.1% | (1%) |
| FPF < 5 μm (%) | | 71.7% | | 67.0% | | 70.0% | |
| FPF < 5 μm (%) range | | 64.8%-78.5% | (6.28%) | 61.2%-77.8% | (6.67%) | 64.8%-76.6% | (4.24%) |
| (stdev) | | | | | | | |
| FPF < 3.3 μm (%) | | 39.8% | | 37.6% | | 38.0% | |
| FPF < 3.3 μm (%) range | | 33.6%-47.8% | (6.59%) | 32.7%-48.9% | (6.70%) | 33.7%-47.7% | (5.53%) |
| (stdev) | | | | | | | |

Overall Device Efficiency

| | | | |
|---|---|---|---|
| Lung dose (FPF × % DD) | 57.4% | Not | 54.3% |
| for 5 μm as % charge | | Determined | |
| Lung dose (FPF × % DD) | 31.9% | | 29.5% |
| for 3.3 μm as % charge | | | |

Table 10 shows the analytical results of nebulized ALTA-2530 using the modified PARI eFlow® Vibrating VM Nebulizer.

TABLE 10
Analytical results of ALTA-2530 in a modified PARI eFlow ® Vibrating Mesh (VM) Nebulizer

| | | 20 mg/mL | | | | 5 mg/mL | | | |
|---|---|---|---|---|---|---|---|---|---|
| | | 4 mL | | | | 4 mL | | | |
| Analytical Results | | Pre-Neb | Post-Ned | Post-Neb | Post-Neb | Pre-Neb | Post-Neb | Post-Neb | Post-Neb |
| Potency (Relative to Reference Standard: | | | Neb 1 | Neb 2 | Neb 3 | | Neb 1 | Neb 2 | Neb 3 |
| iRM lot 11/2) | | | | | | | | | |
| T = 0 Controls - freshly prepared | 1 | 96% | | | | 80% | | | |
| at KBI from Paras DS + diluent | | | | | | | | | |
| Pre-neb samples - controls | 1 | 95% | | | | 85% | | | |
| Post-neb samples - 2 different nebs | 1 | | 95% | 95% | | | 91% | 94% | |
| Content | | Pre-Neb | | | | Pre-Neb | | | |
| A280 (1 mg/mL) | 3 | 19.00 | | | | 4.87 | | | |
| Purity | 1 per | Pre-Neb | Neb 1 | Neb 2 | Neb 3 | Pre-Neb | Neb 1 | Neb 2 | Neb 3 |
| | sample | | | | | | | | |
| SEC (5 mg/mL) × 0.1 mL - | ALTA- | 99.5% | 99.5% | 99.5% | 99.5% | 99.3% | 99.2% | 99.3% | 99.1% |
| 3 different nebs | 2530 | | | | | | | | |
| HMW species, area % (RRT) | | — | 0.14% | 0.14% | 0.12% | — | 0.10% | 0.13% | 0.14% |
| | | 0.12% | (RRT | (RRT | (RRT | 0.11% | (RRT | (RRT | (RRT |
| | | (RRT | 0.61) | 0.61) | 0.61) | (RRT | 0.62) | 0.61) | 0.61) |
| | | 0.90) | — | — | — | 0.90) | — | — | — |
| | | 0.08% | 0.18% | 0.18% | 0.17% | 0.09% | 0.16% | 0.16% | 0.17% |
| | | (RRT | (RRT | (RRT | (RRT | (RRT | (RRT | (RRT | (RRT |
| | | 0.92) | 0.92) | 0.92) | 0.92) | 0.92) | 0.92) | 0.92) | 0.92) |
| LMW species, area % (RRT) | | 0.10% | 0.10% | 0.11% | 0.10% | 0.07% | 0.08% | 0.08 | 0.09 |
| | | (RRT | (RRT | (RRT | (RRT | (RRT | (RRT | (RRT | (RRT |
| | | 1.22) | 1.22) | 1.22) | 1.22) | 1.21) | 1.20) | 1.21) | 1.22) |
| | | 0.08% | 0.09% | 0.08% | 0.08% | 0.37% | 0.37% | 0.37 | 0.46 |
| | | (RRT | (RRT | (RRT | (RRT | (RRT | (RRT | (RRT | (RRT |
| | | 1.27) | 1.27) | 1.27) | 1.27) | 1.24) | 1.25) | 1.25) | 1.26) |
| RP-HPLC - 3 different nebs | ALTA- | 88.5% | 86.3% | 86.6% | 87.1% | 88.3% | 86.6% | 87.7% | 85.4% |
| (area %) | 2530 | | | | | | | | |
| Related Substances. RRT, area % | 0.732- | 0.50 | 0.43 | 0.44 | 0.41 | 0.49 | 0.44 | 0.46 | 0.65 |
| | 0.747 | | | | | | | | |
| | 0.824- | 1.23 | 1.38 | 1.33 | 1.33 | 1.23 | 1.38 | 1.36 | 1.52 |
| | 0.839 | | | | | | | | |
| | 0.860- | 0.29 | 0.73 | 0.74 | 0.86 | 0.29 | 0.76 | 0.68 | 0.87 |
| | 0.881 | | | | | | | | |
| | 0.891- | 0.59 | 1.16 | 1.16 | 1.13 | 0.60 | 1.04 | 0.84 | 0.98 |
| | 0.910 | | | | | | | | |
| | 0.917- | 0.44 | 0.62 | 0.69 | 0.86 | 0.47 | 0.83 | 0.92 | 0.90 |
| | 0.936 | | | | | | | | |
| | 0.963- | 1.64 | 2.14 | 2.18 | 2.00 | 1.66 | 1.96 | 1.89 | 2.35 |
| | 0.977 | | | | | | | | |
| | 1.048- | 1.55 | 1.99 | 1.63 | 1.71 | 1.58 | 1.77 | 1.57 | 1.83 |
| | 1.072 | | | | | | | | |
| | 1.1088- | 1.38 | 1.36 | 1.34 | 1.34 | 1.40 | 1.44 | 1.27 | 1.46 |
| | 1.110 | | | | | | | | |
| | 1.1050- | 1.66 | 1.70 | 1.78 | 1.42 | 1.71 | 1.66 | 1.54 | 1.85 |
| | 1.128 | | | | | | | | |
| | 1.155- | 2.24 | 2.18 | 2.13 | 1.80 | 2.28 | 2.17 | 1.82 | 2.24 |
| | 1.180 | | | | | | | | |
| | Total | 11.52 | 13.69 | 13.42 | 12.86 | 11.71 | 13.44 | 12.35 | 14.64 |
| | RS (%) | | | | | | | | |

Additionally, when using the modified PART eFlow® VM Nebulizer, the ALTA-2530 formulation temperature remained below protein Tm (64-65° C. for rhIL-1ra) which promotes stability. The maximum temperature throughout the aerosol time never reached beyond ˜42° C.

A 150 mg/mL ALTA-2530 drug substance was stored at −80° C. and protected from light. The ALTA-2530 drug substance was thawed overnight at 2-8° C. in the dark until the frozen material melted and became a clear liquid. Accelerated thawing was not conducted. The drug substance and diluent was allowed to equilibrate to room temperature for ˜1 hour prior to dilution. Dilution of the 20 mg/mL and 50 mg/mL ALTA-2530 inhalation solutions was completed using a custom diluent (containing trehalose, EDTA disodium, and histidine in WFI). Clear glass duran bottles covered with foil were used to protect the inhalation solutions from light. The diluted inhalation solutions were stored at 2-8° C. protected from light and were not stored on the benchtop for longer than 8 hours.

### Philips InnoSpire GO Nebulizer

Nebulized 20 mg/mL ALTA-2530 inhalation solution was delivered by Philips InnoSpire GO (ISG) vibrating mesh nebulizer. Testing was performed by non-drug specific methods consisting of droplet size distribution (DSD) by laser diffraction, gravimetric delivered dose (DD), and gravimetric output and residual mass. Three ISG devices were used for testing. ATLA-2530 solution was prepared as follows: DS was removed from 2-8° C. and protect from light and allowed to equilibrate to room temperature for 30 minutes. DS was diluted to 20 mg/mL using Custom Diluent (CD) containing trehalose, EDTA disodium, and histidine in WFI. The formulation was stored at 2-8° C. and protect from light.

Table 11 summarizes the nebulization results for ALTA-2530 in InnoSpire GO.

TABLE 11
Summary results for InnoSpire GO (VMD constant
flow; mean of n = 3 with standard deviation).

| | IL-1RA Inhalation Formulation | | | | | | | | |
|---|---|---|---|---|---|---|---|---|---|
| | | | | | | | Dosing | | |
| Nebulizer | VMD | FPF_{<33 μm} | FPF_{<5.0 μm} | | DD | Exhaust | Time | LOR | Residual |
| ID | (μm) | (%) | (%) | GSD | (g) | (g) | (mins) | (g/min) | (g) |
| SA-031-09 | 4.7 | 27 | 54 | 1.7 | 1.55 | 1.19 ¹ | 7.3 | 0.49 | 0.7 |
| | (0.1) | (1) | (1) | (0.0) | (0.16) | | (0.3) | (0.03) | (0.3) |
| SA-031-06 | 5.1 | 24 | 49 | 1.7 | 1.52 | 0.94 ² | 6.6 | 0.53 | 0.6 |
| | (0.0) | (0) | (0) | (0.0) | (0.07) | | (0.4) | (0.08) | (0.4) |
| SA-009-4 | 4.1 | 36 | 64 | 1.7 | 1.71 | 1.03 ² | 7.5 | 0.47 | 0.6 |
| | (0.0) | (0) | (1) | (0.0) | (0.01) | | (0.2) | (0.01) | (0.0) |

Table 12 summarizes results of nebulizing ALTA-2530 (including anakinra as the IL-1Ra in this embodiment) using the two nebulizers: a modified PARI eFlow® and Philips InnoSpire GO.

TABLE 12
Summary of results of nebulizing ALTA-2530 using two nebulizers

| | | | | | | Aerosol | | | | |
|---|---|---|---|---|---|---|---|---|---|---|
| | | Droplet | | | | Performance | | | | |
| | | Size | Fine | | Liquid | with | | | | |
| | Formulation | Distribution | Particle | Delivered | Output | Repeat Use | Sealed vs | Aerosol | Temp | |
| | Concs | (VMD) | Fraction <3.3 μm | Dose | Rate | (# cycles | Open | Mesh | Profile in | |
| Nebulizer | (mg/mL) | (μm) | (%) | (%) | (mL/min) | tested) | Reservoir | Heads | Reservoir | Controllers |
| PARI | 10 | 3-4 | 47 | 70 | ~0.6 | 14 | Sealed | 3 | up to | 2 |
| eFlow ® | 20 | | | | | | Open | | ~42° C. | (Faster |
| | 50 | | | | | | | | | shut-off) |
| Philips | 20 | 4-5 | 24 | 38 | ~0.5 | 6 | Open | 1 | — | 1 |
| ISG* | | | | | | | | | | |

### Aerogen Solo Nebulizer

Tables 13 and 14 show testing results for ALTA-2530 nebulized with Aerogen Solo Nebulizer. The ALTA-2530 was prepared using custom diluent containing normal saline, NaCl, 154 mM [group 3; diluent 2] or 0.53 mM Disodium EDTA, 300 mM trehalose, and 15 mM histidine [group 5; diluent 1] at 50 mg/mL protein concentration. Tables 13 and 14 shows that there is less than 1% loss of intact protein following nebulization of ALTA-2530.

TABLE 13
Testing results 1 for ALTA-2530 nebulized with Aerogen Solo Nebulizer

| | | | Estimated | Purity | Impurities (% Area) | | |
|---|---|---|---|---|---|---|---|
| | | | Conc ug/mL | (% Area) | HW 1 | HW 2 | LW 1 |
| | Sample | Injection | Provided by | ALTA- | (RRT | (RRT | (RRT |
| Day | Description | Vol (μL) | Lovelace | 2530 | 0.57-0.60) | 0.90-0.92) | 1.15-1.17) |
| Day 1 | Saline/TA FS 1 | 60 | 878.30 | 99.4 | NR | 0.2 | 0.3 |
| (Group 3) | Saline/TA FS 2 | 60 | 745.72 | 98.5 | NR | 1.3 | 1.3 |
| | Saline/TA FS 3 | 60 | 828.21 | 99.1 | NR | 0.2 | 0.6 |
| | Saline/Pre Neb | 1 | 46966.74 | 99.5 | 03 | 0.2 | NR |
| | Saline/Post Neb | 1 | 46994.64 | 99.5 | 0.3 | 0.2 | NR |
| Day 1 | TA + Dil Mid FS1 | 80 | 662.72 | 99.0 | NR | 0.2 | 0.8 |
| (Group 5) | TA + Dil Mid FS2 | 80 | 677.97 | 98.7 | NR | 0.2 | 1.1 |
| | TA + Dil Mid FS3 | 80 | 658.98 | 99.0 | NR | 0.2 | 0.8 |
| | TA + Dil Mid PreNeb | 1 | 58392.63 | 99.6 | 0.2 | 0.2 | NR |
| | TA + Dil Mid | 1 | 58202.87 | 99.6 | 0.2 | 0.3 | NR |
| | PostNeb | | | | | | |
| Day 3 | TA + Sal FS1 | 60 | 841.81 | 98.5 | 0.1 | 0.4 | 1.0 |
| (Group 3) | TA + Sal FS2 | 60 | 733.13 | 98.3 | NR | 0.2 | 1.5 |
| | TA + Sal FS3 | 60 | 715.64 | 97.9 | NR | 0.2 | 1.9 |
| | TA + Sal Pre Neb | 1 | 43132.43 | 99.5 | NR | 0.2 | NR |
| | TA + Sal Post Neb | 1 | 44922.07 | 99. | NR | 0.2 | NR |
| Day 3 | TA + Mid FS1 | 80 | 644.86 | 99.2 | NR | 0.2 | 0.6 |
| (Group 5) | TA + Mid FS2 | 80 | 590.96 | 98.6 | NR | 0.2 | 1.1 |
| | TA + Mid FS3 | 80 | 516.92 | 99.3 | NR | 0.2 | 0.6 |
| | TA + Mid Pre Neb | 1 | 44605.86 | 94.1 | NR | 0.2 | 5.7 |
| | | | | | | | (RRT1.08) |
| | TA + Mid Post Neb | 1 | 45986.45 | 99.8 | NR | 0.2 | NR |
| Day 6 | TA + Sal FS1 | 60 | 777.97 | 99.4 | NR | 0.2 | 0.4 |
| (Group 3) | TA + Sal FS2 | 60 | 797.75 | 98.7 | NR | 0.2 | 1.1 |
| | TA + Sal FS3 | 60 | 777.49 | 99.4 | NR | 0.2 | 0.3 |
| | TA + Sal Pre Neb | 1 | 44860.83 | 99.8 | NR | 0.2 | NR |
| | TA + Sal Post Neb | 1 | 45357.87 | 99.8 | NR | 0.2 | NR |
| Day 6 | TA mid FS1 | 80 | 528.89 | 99.1 | NR | 0.2 | 0.7 |
| (Group 5) | TA mid FS2 | 80 | 514.44 | 98.6 | NR | 0.2 | 1.1 |
| | TA mid FS3 | 80 | 512.71 | 98.9 | NR | 0.2 | 0.9 |
| | TA mid Pre Neb | 1 | 46038.03 | 99.8 | NR | 0.2 | NR |
| | TA mid Post Neb | 1 | 46736.15 | 99.8 | NR | 0.2 | NR |

TABLE 14
Testing results 2 for ALTA-2530 nebulized with Aerogen Solo Nebulizer

| | | | Estimated | Purity | Impurities (% Area) | | |
|---|---|---|---|---|---|---|---|
| | | | Conc ug/mL | (% Area) | HW 1 | HW 2 | LW 1 |
| Day | Sample | Injection | Provided by | ALTA- | (RRT | (RRT | (RRT |
| (Group) | Description | Vol (μL) | Lovelace | 2530 | 0.56-0.64)² | 0.90-0.92) | 1.15-1.17) |
| Day 1 | TA + Sal FS1¹ | 30 | 2081.91 | NA | NA | NA | NA |
| (Group 3) | TA + Sal FS2 | 30 | 1931.87 | 99.6 | 0.3 | NR | 0.1 |
| | TA + Sal FS3 | 30 | 2158.98 | 99.4 | 0.4 | NR | 0.2 |
| | TA + Sal Pre-Neb | 1 | 45957.08 | 99.7 | 0.3 | NR | NR |
| | TA + Sal Post Neb | 1 | 45597.27 | 99.7 | 0.3 | NR | NR |
| Day 1 | TA + Dil1 FS1 | 50 | 927.31 | 100 | NR | NR | NR |
| Group 4, | TA + Dil1 FS2 | 100 | 392.42 | 98.1 | NR | NR | 1.9 |
| 5, 6 | TA + Dil1 FS3 | 50 | 1062.84 | 99.9 | NR | NR | 0.1 |
| | TA + Dil 1 Pre Neb | 1 | 47995.49 | 99.8 | 0.2 | NR | NR |
| | TA + Dil 1 Post Neb | 1 | 47284.42 | 99.8 | 0.2 | NR | NR |
| Day 3 | TA + Sal FS1 (group 3) | 30 | 2066.58 | 98.9 | 0.3 | 0.3 | 0.6 |
| (Group 3) | TA + Sal FS2 (group 3) | 30 | 2019.08 | 98.5 | 0.3 | 0.3 | 1.0 |
| | TA + Sal FS3 (group 3) | 30 | 2141.82 | 98.9 | 0.3 | 0.3 | 0.6 |
| | Pre Neb TA + | 1 | 45195.15 | 99.4 | 0.3 | 0.2 | NR |
| | Sal (group 3) 26 | | | | | | |
| | Post Neb TA + | 1 | 44822.52 | 99.5 | 0.3 | 0.2 | NR |
| | Sal (group 3) | | | | | | |
| Day 3 | TA + Dil FS1 | 60 | 707.32 | 99.1 | NR | NR | 0.9 |
| (Group 4, | TA + Dil FS2 | 60 | 916.67 | 99.6 | NR | NR | 0.4 |
| 5, 6) | TA + Dil FS3 | 60 | 937.66 | 99.4 | NR | NR | 0.6 |
| | TA + Dil Pre Neb | 1 | 46323.59 | 100 | NR | NR | NR |
| | TA + Dil Post Neb | 1 | 46174.38 | 100 | NR | NR | NR |
| Day 6 | TA + Saline FS1 | 30 | 1960.52 | 99.1 | 0.3 | NR | 0.6 |
| (Group 3) | TA + Saline FS2 | 30 | 2063.60 | 99.6 | 0.3 | NR | 0.1 |
| | TA + Saline FS3 | 30 | 2028.25 | 99.3 | 0.3 | NR | 0.4 |
| | TA + Saline Pre Neb | 1 | 48177.38 | 99.8 | 0.2 | NR | NR |
| | TA + Saline Post Neb | 1 | 47969.37 | 99.7 | 0.3 | NR | NR |
| Day 6 | TA + Dil FS1 | 60 | 776.86 | 99.6 | NR | NR | 0.4 |
| (Group 4, | TA + Dil FS2 | 50 | 1068.76 | 99.2 | NR | NR | 0.8 |
| 5, 6) | TA + Dil FS3 | 50 | 1204.37 | 99.5 | NR | NR | 0.5 |
| | TA + Dil Pre Neb | 1 | 46138.23 | 100 | NR | NR | NR |
| | TA + Dil Post Neb | 1 | 54791.98 | 100 | NR | NR | NR |

In some embodiments, when using any of the nebulizers described herein (e.g., the modified PARI eFlow®), the ALTA-2530 formulation temperature remains below protein Tm (64-65° C. for rhIL-1Ra) which promotes stability. In some embodiments, the maximum temperature throughout the aerosol time never reaches beyond ˜42° C.

### Example 8—Examples of ALTA-2530 Formulations

In some embodiments, repeat cycles (up to 14) were simulated of nebulized ALTA-2530 inhalation solution using the modified PARI eFlow® Vibrating Mesh (VM). Characterization included formulation Liquid Output Rate (LOR) and nebulization time, cumulative protein delivered, appearance pre- and post-neb, pH, saline LOR, as a function of nebulizer Aerosol Head (AH). Tables 15-20 show the control and ALTA-2530 formulations that were tested.

TABLE 15
Formulation 1 - 20 mg/mL Histidine Formulation

| Formulation | | | |
|---|---|---|---|
| components | mg/mL | mM | % (w/v) |
| IL-1ra | 20.0 | 1.16 | 2.0 |
| Disodium EDTA | 0.18 | 0.53 | 0.018 |
| Polysorbate 80 | 0.10 | 0.08 | 0.01 |
| Sodium chloride | 1.17 | 20.0 | 0.117 |
| (tonicity adjustment) | | | |
| trehalose | 89.0 | 260.0 | 8.90 |
| histidine | 1.55 | 10.0 | 0.155 |
| Totals | 112.68 | n/a | n/a |

| | |
|---|---|
| Total volume (mL) | 1 |
| Calculated osmolality | 314 |
| (mOsm/kg) | |
| pH | 6.5 |

TABLE 16
Formulation 2 - 20 mg/mL Citrate Formulation

| Formulation | | | |
|---|---|---|---|
| components | mg/mL | mM | % (w/v) |
| IL-1ra | 20.0 | 1.16 | 2.0 |
| Disodium EDTA | 0.18 | 0.53 | 0.018 |
| Polysorbate 80 | 0.10 | 0.08 | 0.01 |
| Sodium chloride | 1.17 | 20.0 | 0.117 |
| (tonicity adjustment) | | | |
| Trehalose | 89.0 | 260.0 | 8.90 |
| Trisodium citrate | 2.36 | 9.15 | 0.236 |
| Citric acid | 1.63 | 0.85 | 0.163 |
| Total solids: mg per mL | 114.4 | n/a | n/a |

| | |
|---|---|
| Total volume (mL) | 1 |
| Calculated osmolality | 330 |
| (mOsm/kg) | |
| pH | 6.5 |

TABLE 17
Formulation 3 - 20 mg/mL Citrate/Phosphate Formulation

| Formulation | | | |
|---|---|---|---|
| components | mg/mL | mM | % (w/v) |
| IL-1ra | 20.0 | 1.16 | 2.0 |
| Disodium EDTA | 0.18 | 0.53 | 0.018 |
| Polysorbate 80 | 0.10 | 0.08 | 0.01 |
| Sodium chloride | 1.17 | 20.0 | 0.117 |
| (tonicity adjustment) | | | |
| Trehalose | 89.0 | 260.0 | 8.90 |
| Sodium phosphate dibasic | 1.18 | 8.30 | 0.118 |
| Citric acid | 0.33 | 1.70 | 0.033 |
| Total solids: mg per mL | 112.0 | n/a | n/a |

| | |
|---|---|
| Total volume (mL) | 1 |
| Calculated osmolality | 327 |
| (mOsm/kg) | |
| pH | 6.5 |

TABLE 18
Formulation 4 - 20 mg/mL Phosphate Formulation

| Formulation | | | |
|---|---|---|---|
| components | mg/mL | mM | % (w/v) |
| IL-1ra | 20.0 | 1.16 | 2.0 |
| Disodium EDTA | 0.18 | 0.53 | 0.018 |
| Polysorbate 80 | 0.10 | 0.08 | 0.01 |
| Sodium chloride | 1.17 | 20.0 | 0.117 |
| (tonicity adjustment) | | | |
| Trehalose | 89.0 | 260.0 | 8.90 |
| Sodium phosphate dibasic | 0.51 | 3.60 | 0.051 |
| Sodium phosphate | 0.77 | 6.40 | 0.077 |
| Total solids: mg per mL | 111.7 | n/a | n/a |

| | |
|---|---|
| Total volume (mL) | 1 |
| Calculated osmolality | 321 |
| (mOsm/kg) | |
| pH | 6.5 |

TABLE 19
Formulation 5 (Positive Control) - 20 mg/mL Formulation

| Formulation | | | |
|---|---|---|---|
| components | mg/mL | mM | % (w/v) |
| IL-1ra | 20.0 | 1.16 | 2.0 |
| Disodium EDTA | 0.18 | 0.53 | 0.018 |
| Polysorbate 80 | 0.07 | 0.05 | 0.007 |
| Citric acid, anhydrous | 0.26 | 1.34 | 0.026 |
| Sodium chloride | 1.11 | 18.9 | 0.111 |
| (tonicity adjustment) | | | |
| trehalose | 89.0 | 260 | 8.9 |
| histidine | 2.01 | 13.0 | 0.201 |
| Totals | 112.68 | n/a | n/a |
| Total volume (mL) | 1 | Diluent | Custom |
| | | | Diluent 1 |

| | |
|---|---|
| Calculated osmolality | 316 |
| (mOsm/kg) | |
| pH | 6.5 |

TABLE 20
Formulation 6 (Negative Control) - Placebo
Matching 20 mg/mL Formulation

| Formulation | | | |
|---|---|---|---|
| components | mg/mL | mM | % (w/v) |
| Disodium EDTA | 0.18 | 0.53 | 0.018 |
| Polysorbate 80 | 0.07 | 0.05 | 0.007 |
| Citric acid, anhydrous | 0.26 | 1.34 | 0.026 |
| Sodium chloride | 1.11 | 18.9 | 0.111 |
| (tonicity adjustment) | | | |
| trehalose | 89.0 | 260 | 8.9 |
| histidine | 2.01 | 13.0 | 0.201 |
| Totals | 112.68 | n/a | n/a |
| Total volume (mL) | 1 | Diluent | Custom |
| | | | Diluent 1 |

| | |
|---|---|
| Calculated osmolality | 316 |
| (mOsm/kg) | |
| pH | 6.5 |

FIG. 8 shows the liquid output rate (LOR, gravimetric mass=g/min) across cycle number (1-14) using the modified PART eFlow® Vibrating Mesh (VM) for formulations 1-6 (see Tables 15-20). ALTA-2530 formulation 1 (containing disodium EDTA, polysorbate 80, sodium chloride, trehalose, and histidine) was the best performing formulation and performed similarly to the negative control (formulation 6, no protein). On the other hand, formulations 2-4 which did not include histidine (but used other buffers) showed worse performance than formulation 1. As shown in FIGS. 8 and 9, formulation 3 testing was performed with two different aerosol heads (AH1 and AH2) in order to assess the impact of AHs with different LORs.

FIG. 9 shows the total protein (μg) output across cycle number (1-14) using the modified PART eFlow® Vibrating Mesh (VM) for formulations 1-6 (see Tables 15-20). FIG. 9 shows that ALTA-2530 formulation 1 (containing disodium EDTA, polysorbate 80, sodium chloride, trehalose, and histidine) was the best performing formulation with the highest total protein output at the end of cycle 14.

FIGS. 10A-10F shows protein diameter (nm) by intensity (%) during testing using a surrogate method (shaking) at 10 minutes, 20 minutes, and 30 minutes across formulations 1-6 (see Tables 15-20). FIGS. 10A-10F shows that for formulation 1 (containing disodium EDTA, polysorbate 80, sodium chloride, trehalose, and histidine) there were no time dependent increases in aggregates. Formulation 6 (negative control) also did not have any time dependent increases in protein aggregates.

Table 21 shows a summary of the aerosol performance of the formulations from Tables 15-20. In some embodiments, the ALTA-2530 formulation (formulation 1) has the following composition: 20 mg/mL IL-1Ra, ˜10 mM histidine buffer, 260 mM trehalose, 20 mM NaCl, 0.53 nM EDTA, ˜0.01% w/v polysorbate 80 at a pH of 6.5. In some embodiments, the composition of ALTA-2530 includes: 20 mg/mL IL-1ra, ˜10 mM histidine buffer, 115 mM trehalose, 20 mM NaCl, 0.53 nM EDTA, ˜0.01% w/v polysorbate 80 at a pH of 6.5.

TABLE 21
Aerosol performance summary for various control and ALTA-2530 formulations

| | | | | | | Max Cycles | Neb Time | | Cum |
|---|---|---|---|---|---|---|---|---|---|
| | | | | Nebulizer | | Completed | (mm:ss) | | Amount |
| | | | Aerosol | Charge | LOR with | before | T = 0 | | of |
| | | Formulation | Head | Volume | saline | clogging (neb | T = | | Protein |
| Form # | Buffer | Prep | Number | (mL | (mL/min) | time > 40 min) | end − 1 | T = end | (mg) |
| 1 | Histidine | Dialysis | SA-305- | 4 | 0.51 | 14 | 7:27 | | 1190 |
| | | Paras 11/6 | 001-14- | | | | 15:41 | | |
| | | | 028-6 | | | | | | |
| 2 | Citrate | Dialysis | SA-305- | 4 | 0.48 | 8 | 9:35 | | 542 |
| | | Paras 11/6 | 001-14- | | | | 10:48 | 40:00 | |
| | | | 028-3 | | | | | | |
| 3 | Citrate/ | Dialysis | SA-305- | 4 | 0.27 | 3 | 17:21 | | 302 |
| | Phosphate | Paras 11/6 | 001-14- | | | | 18:02 | 40:00 | |
| | | | 028-2 | | | | | | |
| | | | SA-305- | 4 | 0.53 | 4 | 8:11 | | 229 |
| | | | 001-14- | | | | 15:59 | 40:00 | |
| | | | 028-6 | | | | | | |
| 4 | Phosphate | Dialysis | SA-305- | 4 | 0.57 | 12 | 8:30 | | 1107 |
| | | Paras 11/6 | 001-14- | | | | 20:11 | 40:00 | |
| | | | 028-3 | | | | | | |
| 5 | Positive | Paras | SA-305- | 4 | 0.46 | 10 | 11:18 | | 677 |
| | Control | 11/6 + CD | 001-14- | | | | 33:44 | 40:00 | |
| | | | 028-1 | | | | | | |

| | | | | | | | | |
|---|---|---|---|---|---|---|---|---|
| 6 | Negative | Paras | SA-305- | 4 | 0.49 | 14 | 7:28 | 0 |
| | Control | formulation | 001-14- | | | | 11:15 | |
| | | buffer + CD | 028-6 | | | | | |

Therefore, it was surprisingly found that using a histidine buffer in ALTA-2530 formulations achieved more nebulization cycles without aggregation (and without clogging) compared to other buffers such as phosphate, phosphate citrate, or citrate. In some embodiments, the concentration of histidine is increased to increase buffer capacity and to reduce a shift in pH with freeze/thaw cycles. In some embodiments, another positively charged amino acid can be used in ALTA-2530 formulations instead of histidine. Other suitable positively charged amino acids include lysine and arginine. Additionally, it was also surprisingly found that formulations having a low (˜0.01% w/v) of polysorbate 80 were shown to mitigate aggregate formation during nebulization.

In some embodiment, ALTA-2530 formulations include a positively charged amino acid. In some embodiments, the positively charged amino acid in the ALTA-2530 formulation is at a concentration of about 5 mM to about 10 mM, about 10 mM to about 15 mM, about 15 mM to about 20 mM, or greater than about 20 mM. In some embodiments the positively charged amino acid in the ALTA-2530 formulation is at a concentration of about 10 mM. In some embodiments the positively charged amino acid in the ALTA-2530 formulation is at a concentration of about 15 mM. In some embodiments, the positively charged amino acid in the ALTA-2530 formulation is histidine.

### Example 9—Inhaled Delivery of ALTA-2530 Achieves Extensive and Prolonged Pulmonary Exposure of rhIL-1Ra Compared to Low Level and Transient Exposure Following Bolus IV Injection

In some embodiments, ALTA-2530 is a novel inhaled formulation of recombinant human IL-1 receptor antagonist (rhIL-1Ra) in development for bronchiolitis obliterans syndrome (BOS). IL-1 overexpression in BOS drives chronic inflammation and fibroblast activation leading to airway remodeling and impaired oxygen transfer. Endogenous IL-1Ra is upregulated in response to IL-1 to limit cytokine signaling, but expression is inadequate to prevent BOS. Pharmacological IL-1 blockade is considered akin to restoration of physiologic immune regulation.

### Purpose:

To determine if ALTA-2530 is stable during nebulization, achieves aerosol particle diameters consistent with distribution to distal airways, and pulmonary exposure commensurate with treatment of BOS.

### Methods:

Aerosolization and in vivo studies were performed with Aerogen Solo or Philips InnoSpire GO vibrating mesh nebulizers. Rats (n=4/grp/timepoint) received ALTA-2530 by nose-only inhalation (0.63, 1.3, and 2.1 mg/g lung). Serum and bronchioalveolar lavage (BAL) samples were collected for analysis by LC-MSMS. ALTA-2530 in lung epithelial lining fluid (ELF) was calculated using a BALF dilution factor. BALF dilution factor was calculated based on the methods disclosed in Rennard S I, Basset G, Lecossier D, O'Donnell K M, Pinkston P, Martin P G, Crystal R G. Estimation of volume of epithelial lining fluid recovered by lavage using urea as marker of dilution. Journal of Applied Physiology. 1986 Feb. 1; 60(2):532-8.

Inhaled delivery of ALTA-2530 achieves extensive, stable, and sustained exposure in lung epithelial lining fluid that in rodents markedly exceeds 24 hr, in contrast to exposure following bolus IV delivery where exposure is transient and <20 min. Lung is the target organ for treatment of conditions including, but not limited to: post lung transplant conditions including BOS, primary graft dysfunction (PGD), reperfusion injury, infection related ARDS, or chemical lung injury. Achieving pharmacologically relevant levels of rhIL-1Ra in lung tissue requires high-dose SC or IV treatment with rhIL-1Ra resulting in renal impairment and neutropenia in some patients. IV delivery provide low level and transient exposure to lung tissue. Inhaled delivery targets the organ of clinical significance and achieves long lasting high exposure levels.

Recombinant human IL-1 receptor antagonist (rhIL-1Ra) exposure was determined in lung bronchioalveolar lavage fluid (BALF) following inhaled delivery to male and female Sprague Dawley rats.

Sprague Dawley male (M) and female (F) rats were weighed and randomized into study groups (Table 22). One group was kept naïve, all other animals were exposed to a single dose of either the Vehicle (normal saline, 0.9% sodium chloride), or to ALTA-2530 test article (TA) recombinant human IL-1 receptor antagonist (rhIL-1Ra) via nose-only inhalation. Target dose levels of rhIL-1Ra were regulated by exposure duration at a target aerosol concentration of 1.5 milligrams (mg)/liter (L).

TABLE 22
Experimental Design

| | | | | Target Necropsy Post Exposure | |
|---|---|---|---|---|---|
| | | | | 5, 30 minutes, | |
| | Presented Dose | | | 1, 2, 3, 4, 6, 8, | |
| | (Delivered Dose) | Exposure Time | | 12 hours | 24, 72 hours |
| Group | mg/kg | (min) | n | (2M/2F per group) | (3M/3F per group) |
| 1 | Treatment | N/A | 3M/3F | N/A | 24 hour: |
| | Naïve | | | | TK serum, BALF, |
| | | | | | lung tissue |
| 2 | 53.5 + 1.5 | 180 | 3M/3F | TK serum 1 hr | 24 hour: |
| | (5.35 ± 0.15) | | | post-exposure | TK serum, BALF, |
| | | | | (non-terminal, | lung tissue |
| | | | | 3M/3F) | |
| 3 | 33.5 ± 0.08 | 60 | 24M/24F | TK serum, BALF, | TK serum, BALF, |
| | (3.35 ± 0.08) | | | lung tissue | lung tissue, |
| | | | | | Clinical pathology |
| 4 | 70.7 ± 1.8 | 120 | 24M/24F | TK serum, BALF, | TK serum, BALF, |
| | (7.07 ± 0.18) | | | lung tissue | lung tissue, |
| | | | | | Clinical pathology |
| 5 | 111.5 ± 4.2 | 180 | 24M/24F | TK serum, BALF, | TK serum, BALF, |
| | (11.2 ± 0.4) | | | lung tissue | lung tissue, |
| | | | | | Clinical pathology |

N/A = not applicable

Blood (serum) and BALF were collected for toxicokinetic (TK) analysis from all TK animals during scheduled necropsy following exposures.

Serum and BALF levels of rhIL-1Ra were determined by means of affinity capture LC-MSMS. rhIL-1RA was captured from serum and BALF samples using streptavidin magnetic beads coated with anti-human IL-1RA antibody, subjected to “on-bead” proteolysis with trypsin, denatured, reduced, and alkylated, resulting in characteristic peptide fragments originating from rhIL-1RA. A selected characteristic peptide was quantified as a surrogate of the ALTA-2530 concentrations in samples.

Concentrations of rhIL-1Ra in BALF were corrected for the dilution factor introduced during collection of epithelial lining fluid (ELF) using normalization of BALF and plasma urea as described by Rennard et al., J. Applied Physiol., 1986. Levels of urea in BALF were less than the lower limit of quantitation (LLOQ) so the normalization factor was calculated using the LLOQ value (1 mg/dL). Thus, the reported values for rhIL-1Ra in ELF are likely an under-estimate of true concentration. Mean plasma urea concentrations were used based on combined gender groups mean values for plasma urea.

Epithelial lining fluid (ELF) concentrations were calculated from serum-urea corrected bronchioalveolar lavage fluid concentrations.

### Results:

Inhaled delivery of ALTA-2530 achieves prolonged pulmonary exposure of rhIL-1Ra that exceeded 24 hr in rat compared to transient exposure of <20 min following bolus IV injection. This is predictive for once or twice daily, or even less frequent, dosing clinically compared to multiple daily IV doses required for the treatment of lung pathologies. In contrast, it is known that IV administration provides only transient lung exposure. Therefore, the prolonged lung exposure beyond 24 hours (e.g., 48 hours) achieved using ALTA-2530 is surprising and unexpected. Moreover, the ratio of lung epithelial lining fluid to plasma exposures in rats were >2500-fold compared to 0.44-fold for lung tissue: plasma following a 5 hr IV infusion.

Nebulized ALTA-2530 delivered rhIL-1Ra particles with mass median aerodynamic diameters of ˜2.5-4 μm, consistent with delivery to small bronchioles. Impurity profiling by HPLC-UV and HPLC-SEC methods and an in vitro potency assay demonstrated rhIL-1Ra protein was stable during nebulization and retained full potency.

Descriptive pharmacokinetic parameters for rhIL-1Ra in serum and ELF are presented in Table 23. FIG. 11 shows concentration versus time profiles for rhIL-1Ra in ELF and serum following single doses to rat using the Aerogen Solo nebulizer.

TABLE 23
Descriptive pharmacokinetic parameters in serum and ELF following
single doses of ALTA-2530 to rat using Aerogen Solo

| Group #, | Dose ^{a} | | Tₘₐₓ | Cₘₐₓ | AUCₗₐₛₜ | AUC_{0-inf} | t_{1/2} |
|---|---|---|---|---|---|---|---|
| Dose level | (mg/kg) | Sample | (h) | (ng/mL) | (h*ng/mL) | (h*ng/mL) | (h) |
| 3, Dose 1 | 3.34 | Serum | 4.0 | 202 | 1785 | — | 4.38 |
| | | ELF | 0.08 | 754,000 | 4,990,000 | 5,430,000 | 6.36 |
| 4, Dose 2 | 7.07 | Serum | 0.08 | 400 | 3,040 | — | 4.77 |
| | | ELF | 0.08 | 1,400,000 | 9,790,000 | 1,400,000 | 5.48 |
| 5, Dose 3 | 11.20 | Serum | 0.50 | 626 | 4,525 | — | 4.14 |
| | | ELF | 0.08 | 1,920,000 | 12,500,000 | 13,700,000 | 4.21 |

^{a} Dose is average pulmonary deposited dose based on terminal body weight with deposition
— Not calculated

The IC₅₀ value as determined by the PathHunter® Anakinra Bioassay Kit was 0.6 μg/mL (or 600 ng/mL). Based on this, lung exposures following single doses in rat were >29-fold the IC₅₀ of ALTA-2530. These values likely represent an underestimate as urea was <1 mg/dL in BALF. These values likely represent an underestimate as urea was <1 mg/dL in BALF. Following 7 days of repeat dosing, the ELF exposures 24 hrs post dose were 6 to 18-fold (depending on exposure time) above the IC₅₀ of ALTA-2530.

A whole blood IL-6 release assay in rats showed that the IC₅₀ was 399 ng/mL. Based on this, the 7-day study low dose results showed a 9-fold higher ELF exposure above the IC₅₀ of ALTA-2530 and the 7-day high dose results showed a 26-fold higher ELF exposure above the IC₅₀ of ALTA-2530.

### Discussion:

Inhaled delivery of ALTA-2530 achieved prolonged pulmonary exposure of rhIL-1Ra that exceeded 24 hr in rat compared to transient exposure of <20 min following bolus IV injection.

The prolonged exposure of rhIL-1Ra in lung following inhaled delivery of ALTA-2530 is predictive for once or twice daily, or less frequent, dosing clinically compared to multiple daily IV doses likely required for the treatment of lung pathologies where lung exposure would be transient.

The ratio of lung epithelial lining fluid to serum exposures as AUC were >2500-fold across all inhaled doses compared to 0.44-fold for lung tissue: plasma following a 5 hr IV infusion.

IL-1Ra binds to the IL-1RI receptor with comparable affinity as IL-1β; thus, if comparable rhIL-1Ra levels ˜100× levels are needed for pharmacological levels in lung tissue. Table 24 shows surface plasmon resonance binding studies indicating that rhIL1-Ra (in an ALTA-2530 composition) binds to the IL-1 type 1 receptor with ˜100 fold higher affinity than IL-1β and ˜10,000 fold affinity than IL-la. At human equivalent doses (based on mg/g lung) rat BALF rhIL-1ra concentrations exceeded those of IL-1β reported in BAL of BOS patients by >1000×.

TABLE 24
Binding of ALTA-2530, IL-1β, IL-1α,
and an IL-1Ra to IL-1 type 1 receptor

| | kₐ | k_{d} | K_{D} |
|---|---|---|---|
| | “on” | “off” | Equilibrium |
| Test ligand | (1/ms) | (1/s) | (M) |
| ALTA-2530 | 2.46E+06 | 1.80E−05 | 7.43E−12 |
| rhlL-1α | 2.65E+03 | 1.45E−03 | 5.47E−07 |
| rhlL-1β | 1.80E+07 | 3.46E−03 | 1.92E−10 |
| IL-1Ra | 8.54E+05 | 3.91E−05 | 5.0E−11 |

In some embodiments, nebulized ALTA-2530 delivers stable and active rhIL-1Ra protein, in a particle size for delivery to small airways of the lung and exposure duration predictive for once daily therapeutic dosing in BOS.

Effective animal doses from in vivo studies (e.g., see Table 2 above) can be converted to appropriate human doses using conversion methods known in the art (e.g., see Tepper et al, Breath in, breath out, it's easy: What you need to know about developing inhaled drugs”, Int J of Tox, 2016 35(4) 376-392). In some embodiments, the rat dose can be converted to human dose based on mg of ALTA-2530 per g of lung weight. In some embodiments, human patients are administered inhaled ALTA-2530 at doses of between about 0.5 mg/kg to about 2 mg/kg.

### Example 10—Inhaled ALTA-2530 Delivery of IL-1Ra to Target Regions of Lung in Animal Studies

FIGS. 12A-12B shows immunohistochemistry staining images of non-human primate lung for rh-IL-1Ra conducted using a commercially available anti-IL-1Ra antibody. The images show positive staining of the epithelium of the bronchioles, the alveolar septae, and the smooth muscle layer of the arteries (4× magnified). Non-human primates (cynomolgus monkeys) were administered via nebulization (using the Aerogen Solo) either saline or ALTA-2530 (0.86 mg/g lung weight) once daily for seven days. FIG. 12A shows immunohistochemistry staining of lung tissue of a non-human primate that received saline and FIG. 12B shows immunohistochemistry staining lung tissue of a non-human primate that received nebulized ALTA-2530 (0.86 mg/g lung weight). FIG. 12B shows that ALTA-2530 reaches bronchioles deep in the lung (suggesting formulated particle size is effective) and effectively coats bronchioles (target tissue). Nebulized ALTA-2530 delivered to small airways of the lung of non-human primates sustained pharmacologically relevant levels of rhIL-1Ra protein and was well-tolerated in acute dose and 7-day repeat dose toxicity studies demonstrating promise for therapeutic dosing in chronic lung allograft dysfunction. ALTA-2530 rhIL-1Ra was shown to be stable and retained potency after nebulization. ALTA-2530 rhIL-1Ra was shown to be stable in lung ELF. ALTA-2530 formulation achieved extensive and prolonged exposure in ELF that at trough 24 hr after dosing, was >29-fold the rhIL-1Ra IC₅₀ (commercially available IL-1Ra potency assay was used for IC₅₀ determination). The Mass Medium Aerodynamic Diameter (MMAD) from rodent study ranged from 2.18 to 3.19 μm. Thus, inhaled ALTA-2530 delivered IL-1Ra to small airways in nonhuman primates consistent with treatment target for BOS. At about 0.4 hours post single exposure, the ELF exposure levels were 540-fold above the IC₅₀ when using the DiscoverX kit and 46,500-fold above IC₅₀ using a whole blood IL-6 assay.

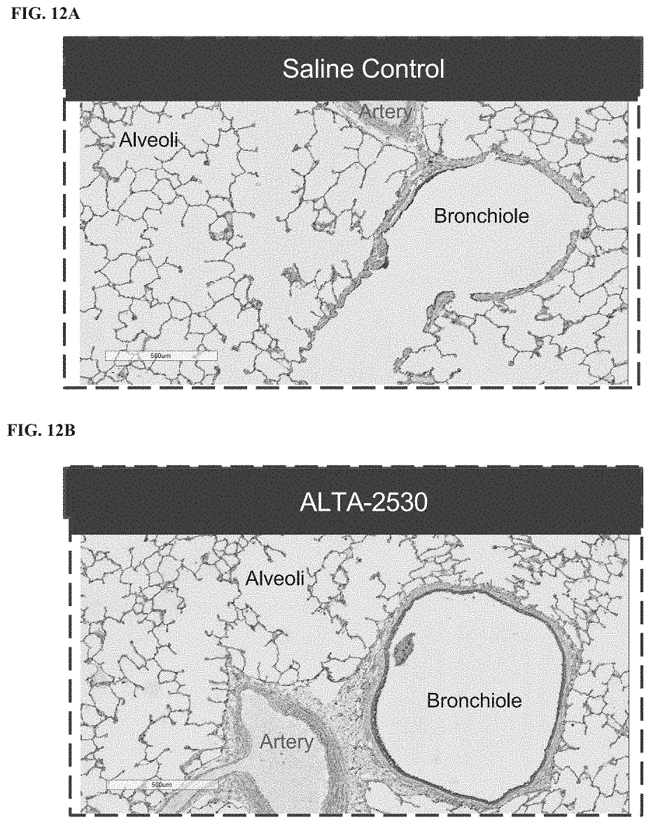

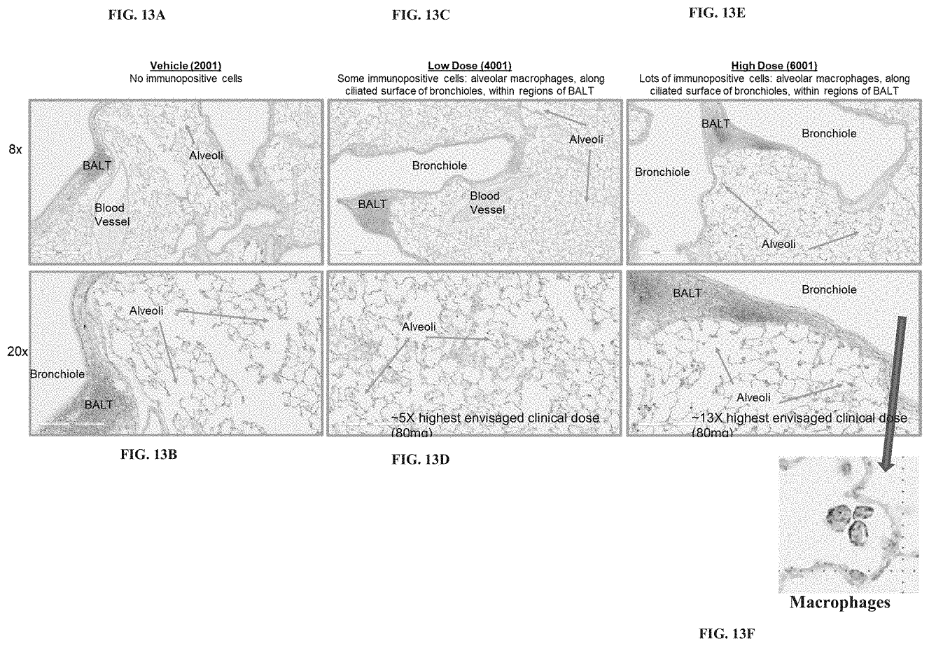

In another study, rats received once-daily exposure to saline or ALTA-2530 via the Aerogen Solo nebulizer for 7 days. Tissues were collected 24 hr after the last dose (trough exposures). FIG. 13 shows immunohistochemistry staining images of rat lung tissue using a novel antibody that reduced non-specific staining in rat tissue. The images confirm exposure in bronchioles and alveolar macrophages (AM). The images show rat lung after exposure to nebulized vehicle (FIG. 13A, 8 times magnified; FIG. 13B, 20 times magnified), after exposure to ALTA-2530 at a low dose (presented dose of 20.4±7.5 mg/kg; (deposited dose of 2.04±0.75 mg/kg)) (FIG. 13C, 8 times magnified; FIG. 13D, 20 times magnified), and after exposure to ALTA-2530 at a high dose (56.4±6.1 (5.64±0.61)) (FIG. 13E, 8 times magnified; FIG. 13F, 20 times magnified). Lung exposed to vehicle showed no immunopositive cells (e.g., alveolar macrophages), lung exposed to low dose ALTA-2530 showed some immunopositive cells (e.g., alveolar macrophages), and lung exposed to high dose ALTA-2530 showed many immunopositive cells (e.g., alveolar macrophages).

### Example 11—ALTA-2530 Sulfur Mustard Challenge Study in Rat

The rat study included a saline group and nebulized ALTA-2530 treatment group in which the dose duration was 60 minutes daily. Sulfur mustard (SM) dose was selected based on historical data to target 50% mortality at post challenge day 28. Instead LD50 was reached at Day 10 suggesting: Sulfur mustard dose in this study appears to be greater than an LD50, or deeper administration heightened effects. Other factors, e.g., stress of nose cone dosing exacerbated morbidity in already frail animals. Histopathology data for rats treated for 10 days suggests treatment benefit for ALTA-2530. Data includes survival, pulse oximetry, SM dose analysis, and histopathology. Table 25 shows mortality compared across treatment groups for total analysis set and statistical analysis set (Kaplan Meier survival plots). The total analysis set comprises all unscheduled deaths, but not scheduled deaths as these were included to characterize progression of lung injury. Total analysis set includes groups 1-7 and statistical analysis set includes groups 6 and 7 in Table 25.

TABLE 25
Summary data from Kaplan Meier survival plots

| | | | | Saline or | |
|---|---|---|---|---|---|
| | | SM | | ALTA-2530 | |
| | Number | Challenge | | Dose | |
| | of Male | Exposure | | duration | Day of |
| Group | Animals | Level | Treatment* | (Days) | Euthanasia |
| 1 | 8 | Air only | 0.9% Saline | 28 | 29 |
| 2 | 4 | LD50/ | 0.9% Saline | 2 | 3 |
| 3 | 4 | 28 day | Nebulized | 2 | 3 |
| | | | ALTA-2530 | | |
| 4 | 4 | | 0.9% Saline | 7 | 8 |
| 5 | 4 | | Nebulized | 7 | 8 |
| | | | ALTA-2530 | | |
| 6 | 16 | | 0.9% Saline | 28 | 29 |
| 7 | 16 | | Nebulized | 28 | 29 |
| | | | ALTA-2530 | | |

All SM challenged animals showed severe hypoxemia (marked reduction in oxygen saturation) without evidence of recovery post challenge. Values under 90% can lead to serious deterioration; 70% is life threatening. Thus, the study was terminated at day 10. In any case, histopathology had multiple endpoints to evaluate necrosis (study terminated too early to evaluate repair and fibrosis). A treatment effect for ALTA-2530 was surprisingly observed in reducing necrosis. FIGS. 14A-14B shows necrosis (0=no necrosis, 5=sever necrosis based on a 0-5 semi-quantitative measurement scale) in the respiratory epithelium (FIG. 14A) and in the bronchial epithelium (FIG. 14B) for saline treated rats and for ALTA-2530 treated rats (shown as SM:2530 in FIG. 14) using the Aerogen Solo nebulizer. FIG. 14 shows that for both the respiratory epithelium and the bronchial epithelium, ALTA-2530 treated rats had less necrosis compared to saline treated control rats.

Without being bound by any specific theory, it is believed that ALTA-2530 blocks necrosis by controlling caspase expression or activity. IL-1Ra blocks IL-1α and IL-1β. IL-1α is implicated in caspase1 expression, and caspase 1 drives necrosis. It is possible that ALTA-2530 neutralizes extracellular IL-1α by blockade of IL-1R1, which in turn markedly reduces the induction of procaspase-1 expression.

### Example 12—ALTA-2530, an Inhaled rhIL-1Ra, Demonstrates Distribution to Distal Regions of Lung and High Affinity IL-1 Receptor Blockade Consistent with Development as a Treatment for Bronchiolitis Obliterans Syndrome

### Purpose

Dysregulated expression of interleukin-1 (IL-1), and downstream cytokines, has been implicated in the development and progression of bronchiolitis obliterans syndrome. Quiescence of IL-1 signaling via blockade of the IL-1 receptor type 1 (IL-1R1) is proposed to restore physiologic immune regulation. Herein, we characterize: i) distribution of ALTA-2530 in rodent and non-human primate (NHP) lung tissue following aerosolized administration, ii) binding affinity to IL-1R1, and iii) suppression of downstream IL-6 expression to assess potential for receptor blockade and development of an exposure response to guide dose selection for in vivo studies.

### Methods

Bronchoalveolar lavage (BAL) and lung tissues samples were collected from rat and NHP following 7 daily inhaled doses of ALTA-2530 using the Aerogen Solo nebulizer. ALTA-2530 was determined in BAL by affinity capture LC-MSMS. Tissues were processed for immunohistochemistry and rhIL-1Ra localized using either commercial (NHP) or affinity purified (rat) polyclonal antibodies. Affinity purification was performed to enhance selectivity for rhIL-1Ra over endogenous protein. Binding kinetics of rhIL-1Ra to human IL-1R1 were determined by surface plasmon resonance and compared to IL-1α and (3. ALTA-2530 mediated inhibition of IL-6 expression in whole blood across species was determined following stimulation by IL-1β.

### Results

Once daily administration of ALTA-2530 provided >24 hr exposure in BAL. Immunohistochemistry of lungs from healthy rats and NHPs 24 hr post dose demonstrated dose-dependent delivery of ALTA-2530 to alveoli and bronchial epithelial cells. Early data also indicate association with alveolar macrophages. Distribution to narrow rat bronchioles is supportive of therapeutically relevant distribution in human bronchioles—a target tissue for the treatment of BOS. ALTA-2530 IL-1Ra bound to IL-1R1 with over 100× greater affinity (K_{D}˜10¹² M⁻¹, k_{d}˜10⁵ s⁻¹, kₐ˜10⁶ M⁻¹s⁻¹) than endogenous IL-1 agonists IL-1α ((K_{D}˜10⁷ k_{d}˜10³ s⁻¹, k_{d}˜10³ M⁻¹s⁻¹) and IL-1β (K_{D}˜10¹⁰ k_{d}˜10³ s⁻¹, kₐ˜10⁷ M⁻¹s⁻¹) (see Table 24).

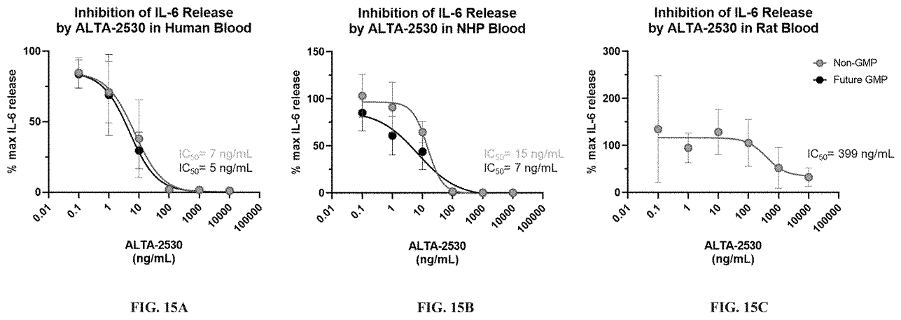

Functional potency of IL-1R1 blockade in fresh whole blood demonstrated rhIL-1Ra inhibited IL-6 expression following IL-1β stimulation, supportive of a therapy for IL-1 and IL-6 driven pathologies including BOS. Whole blood from healthy human, NHP, and rat donors (n=3, n=6 for rats) was incubated with IL-11β for 24 hours to induce IL-6 release (quantified using ELISA). FIGS. 15A-15C shows ALTA-2530 titrated to determine IC₅₀ values (concentration of ALTA-2530 by % max IL-6 release) for human (FIG. 15A), NHP (FIG. 15B), and rat (FIG. 15C). FIGS. 15A-15C shows that ALTA-2530 inhibits IL-6 release in human and NHP whole blood.

### Conclusion

ALTA-2530 delivers potent rhIL-1Ra to distal regions of lung, consistent with a therapy for BOS.

### Example 13—Assessing the Safety, Pharmacological and Biochemical Effects of Nebulized Anakinra in Healthy Smokers

An exploratory, single-dose, dose-escalating phase 1 study was performed with 18 healthy smokers. All 18 subjects received nebulized inhalation of anakinra. The subjects were separated into three (3) dose groups, and dosage forms of anakinra were administered as follows: six (6) subjects received a dosage level of 0.75 mg, six (6) subjects received a dosage level of 3.75 mg, and six (6) subjects received a dosage level of 7 mg. There was a 14-day interval between each successive dose group whereby the safety of four (4) subjects in the prior dose group was assessed. Safety assessment included physical examinations, vital sign measurements, clinical laboratory evaluations, documentation of AEs, electrocardiogram (ECG) assessments, and pulmonary function (FEV1), forced expiratory flow (FEF) of 25-75%, and forced vital capacity (FVC). Bronchoscopy for pharmacologic and biochemical endpoint was performed on two (2) subjects within each dose group, after the four (4) subjects in each dose group were analyzed for safety. Bronchoscopic analysis was carried out independent of the safety analysis. The total duration of the study was 2.5 months.

### Example 14—Content Uniformity

This test is designed to demonstrate the uniformity of medication per spray (or minimum dose), consistent with the label claim, to be discharged from the actuator or mouthpiece, of an appropriate number (n=about 10 from beginning and n=about 10 from end) of containers from a batch. The primary purpose is to ensure spray content uniformity within the same container and among multiple containers of a batch. Techniques for thoroughly analyzing the spray discharged from the actuator or mouthpiece for the drug substance content include multiple sprays from beginning to the end of individual container, among containers, and among batches of drug product. This test provides an overall performance evaluation of a batch, assessing the formulation, the manufacturing process, and the pump. At most, two sprays per determination are used except in the case where the number of sprays per minimum dose specified in the product labeling is one. To ensure reproducible in vitro dose collection, the procedure will have controls for actuation parameters (e.g., stroke length, actuation force). The test is performed with units primed following the instructions in the labeling. The amount of drug substance delivered from the actuator or mouthpiece is expressed both as the actual amount and as a percentage of label claim.

The following acceptance criteria are used. However, alternative approaches (e.g., statistical) may be used to provide equal or greater assurance of spray content uniformity. In general, for acceptance of a batch (1) the amount of active ingredient per determination is not outside of 80 to 120 percent of label claim for more than 2 of 20 determinations (10 from beginning and 10 from end) from 10 containers, (2) none of the determinations is outside of 75 to 125 percent of the label claim, and (3) the mean for each of the beginning and end determinations are not outside of 85 to 115 percent of label claim.

### Example 15—Proposed Clinical Studies of ALTA-2530 in BOS

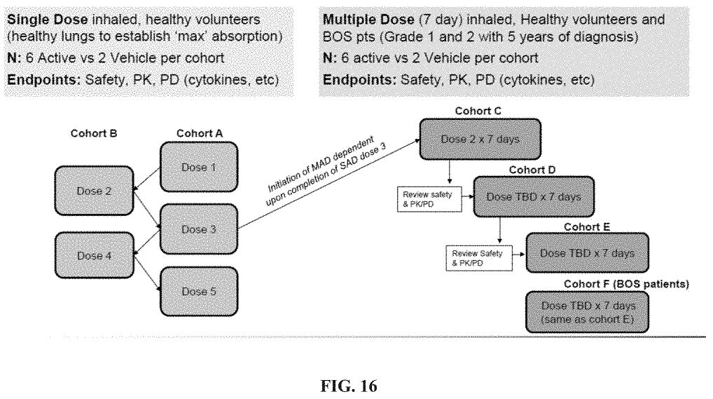

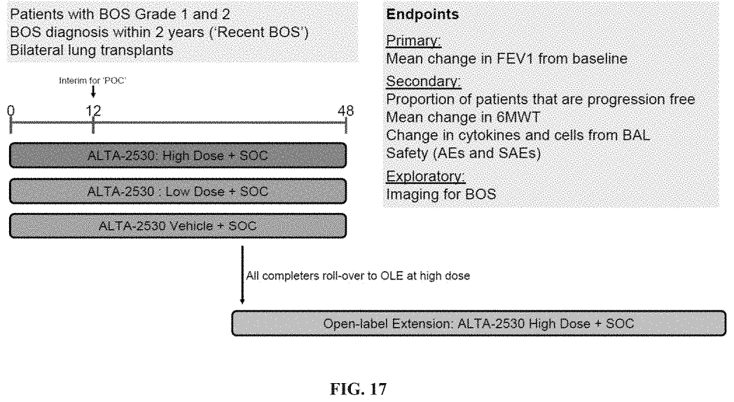

FIG. 16 shows a phase 1 study using single/multiple ascending doses (SAD/MAD) of ALTA-2530 in healthy volunteers and BOS patients. The proposed study would be conducted as single trial, with a MAD limit of 7 days in healthy volunteers and BOS patients.

FIG. 17 shows a phase 2b/3 pivotal study of ALTA-2530 in BOS patients with 12-week proof of concept interim.

1. A pharmaceutical composition comprising:
an interleukin-1 receptor antagonist,
wherein the interleukin-1 receptor antagonist is a protein or a peptide;
a buffer; and
optionally one or more additional components each selected from the group consisting of a stabilizer and a tonicity modifier,
wherein the pharmaceutical composition is adapted for administration via inhalation.

2. The pharmaceutical composition of claim 1, wherein the buffer comprises an amino acid or phosphate.

3. The pharmaceutical composition of claim 1, wherein the buffer comprises a positively charged amino acid.

4. The pharmaceutical composition of claim 3, wherein the positively charged amino acid is selected from the group consisting of lysine, arginine, and histidine.

5. The pharmaceutical composition of claim 4, wherein the positively charged amino acid is histidine.

6. The pharmaceutical composition of claim 1, wherein the buffer is selected from the group consisting of citrate, phosphate, succinate, histidine, lysine, arginine, glutamate, pyrophosphate, 4-(2-hydroxyethyl)-1-piperazineethanesulfonic acid (HEPES), and a combination thereof.

7. The pharmaceutical composition of claim 1, wherein the buffer comprises histidine or phosphate.

8. The pharmaceutical composition of claim 6, wherein the pharmaceutical composition is a liquid composition comprising histidine in a concentration of between about 5 mM and 50 mM.

9. The pharmaceutical composition of claim 8, wherein the concentration of histidine is about 5, 10, 15, 20, 25, 30, 35, 40 or 45 mM.

10. The pharmaceutical composition of claim 9, wherein the concentration of histidine is about 10 mM.

11. The pharmaceutical composition of claim 6, wherein the pharmaceutical composition is a liquid composition comprising phosphate in a concentration of between about 1 mM and 50 mM.

12. The pharmaceutical composition of claim 11, wherein the concentration of phosphate is about 5, 10, 15, 20, 25, 30, 35, 40 or 45 mM.

13. The pharmaceutical composition of claim 12, wherein the concentration of phosphate is about 10 mM.

14. The pharmaceutical composition of claim 1, wherein the pharmaceutical composition further comprises a stabilizer selected from the group consisting of a surfactant, a chelating agent, a sugar, and a combination thereof.

15. The pharmaceutical composition of claim 14, wherein the stabilizer is a non-reducing sugar.

16. The pharmaceutical composition of claim 15, wherein the non-reducing sugar is selected from the group consisting of trehalose, sucrose, glycerol, sorbitol, and a combination thereof.

17. The pharmaceutical composition of claim 16, wherein the non-reducing sugar is trehalose.

18. The pharmaceutical composition of claim 15, wherein the pharmaceutical composition is a liquid composition, and the concentration of the non-reducing sugar is greater than about 5% (w/v).

19. The pharmaceutical composition of claim 14, wherein the pharmaceutical composition is a liquid composition comprising trehalose in a concentration of between about 100 mM and 350 mM.

20. The pharmaceutical composition of claim 19, wherein the concentration of trehalose is about 115 mM.

21. The pharmaceutical composition of claim 14, wherein the stabilizer is a chelating agent which is ethylenediaminetetraacetic acid (EDTA) disodium.

22. The pharmaceutical composition of claim 21, wherein the pharmaceutical composition is a liquid composition comprising ethylenediaminetetraacetic acid (EDTA) disodium in a concentration of between about 0.05 mM and 1 mM.

23. The pharmaceutical composition of claim 22, wherein the concentration of ethylenediaminetetraacetic acid (EDTA) is about 0.53 mM.

24. The pharmaceutical composition of claim 14, wherein the stabilizer is a surfactant selected from the group consisting of polysorbate 80, polysorbate 20, polyoxyethylene (23) lauryl ether (Brij™ 35), sorbitan trioleate (Span™ 85), and a combination thereof.

25. The pharmaceutical composition of claim 24, wherein the pharmaceutical composition is a liquid composition comprising polysorbate 80 in a concentration of between about 0.001% and 1% (w/v).

26. The pharmaceutical composition of claim 24, wherein the concentration of polysorbate 80 is between about 0.05% and 0.035% (w/v).

27. The pharmaceutical composition of any of claim 1, wherein the pharmaceutical composition further comprises a tonicity modifier.

28. The pharmaceutical composition of claim 27, wherein the tonicity modifier is selected from the group consisting of sodium chloride, mannitol, taurine, hydroxyproline, proline, and a combination thereof.

29. The pharmaceutical composition of claim 27, wherein the pharmaceutical composition is a liquid composition comprising sodium chloride in a concentration of between about 10 mM and 50 mM.

30. The pharmaceutical composition of claim 28, wherein the concentration of sodium chloride is about 20 mM.

31. The pharmaceutical composition of claim 1, wherein the pharmaceutical composition comprises: a buffer comprising an amino acid or phosphate; and a stabilizer comprising a non-reducing sugar.

32. The pharmaceutical composition of claim 31, wherein the composition comprises: a buffer comprising histidine or phosphate; and a stabilizer comprising trehalose.

33. The pharmaceutical composition of claim 32, wherein the composition comprises: histidine; trehalose; sodium chloride; polysorbate 80; and ethylenediaminetetraacetic acid (EDTA) disodium.

34. The pharmaceutical composition of claim 1, wherein the composition comprises: phosphate; trehalose; sodium chloride; polysorbate 80; and ethylenediaminetetraacetic acid (EDTA) disodium.

35. The pharmaceutical composition of any of claim 1, wherein the pH of the liquid composition is between about 6 and 8.

36. The pharmaceutical composition of any of claim 1, wherein the pH of the liquid composition is about 6.5.

37. The pharmaceutical composition of any of claim 1, wherein the osmolality of the liquid composition is between about 200 mOsm/kg and 400 mOsm/kg.

38. The pharmaceutical composition of any of claim 1, wherein the osmolality of the liquid composition is about 300 mOsm/kg.

39. The pharmaceutical composition of any of claim 1, wherein the pharmaceutical composition is a liquid composition comprising the interleukin-1 receptor antagonist in a concentration of between about 1 mg/mL and 30 mg/mL.

40. The pharmaceutical composition of claim 39, wherein the concentration of the interleukin-1 receptor antagonist is about 5 mg/mL.

41. The pharmaceutical composition of claim 39, wherein the concentration of the interleukin-1 receptor antagonist is about 20 mg/mL.

42. The pharmaceutical composition of claim 1, wherein the interleukin-1 receptor antagonist is anakinra.

43. A kit comprising the pharmaceutical composition of claim 1 and a delivery device suitable for direct administration of the pharmaceutical composition to the respiratory tract of a patient.

44. The kit of claim 43, wherein the respiratory tract comprises the lower airways.

45. The kit of claim 44, wherein the delivery device is configured to deliver an effective amount of the pharmaceutical composition via inhalation.

46. The kit of any of claim 43, wherein the delivery device is selected from the group consisting of a nebulizer, an inhaler, and an aerolizer.

47. The kit of claim 46, wherein the delivery device is selected from the group consisting of a jet nebulizer, a mesh nebulizer, an ultrasonic nebulizer, a metered dose inhaler, and a dry powder inhaler.

48. The kit of claim 47, wherein the nebulizer is selected from the group consisting of the Aerogen Solo and the AeroEclipse II nebulizers.

49. The kit of claim 48, wherein the nebulizer is the Aerogen Solo.

50. The kit of any of claim 44, wherein the droplet size of the liquid composition produced by the delivery device is between about 0.5 μm and 10 μm in diameter.

51. The kit of claim 50, wherein the droplet size of the liquid composition produced by the delivery device is between about 2.5 μm and 4 μm in diameter.

52. The kit of claim 51, wherein the droplet size of the liquid composition produced by the delivery device is about 3.5 μm in diameter.

53-77. (canceled)
